(19) United States
(12) Patent Application Publication
(10) Pub. No.: US 2009/0297548 A1
(43) Pub. Date: Dec. 3, 2009

(54) IMMUNOGENIC COMPOSITIONS COMPRISING ANTHRAX SPORE-ASSOCIATED PROTEINS

(75) Inventors: Indira T. Kudva, Newberry, FL (US); Stephen B. Calderwood, Wellesley, MA (US); Manohar John, Newberry, FL (US)

Correspondence Address: EWARDS ANGELL PALMER & DODGE LLP, P.O. BOX 55874, BOSTON, MA, 02205, US

(73) Assignee: THE GENERAL HOSPITAL CORPORATION, Boston, MA (US)

(21) Appl. No.: 11/996,160

(22) PCT Filed: Jul. 19, 2006
(86) PCT No.: PCT/US06/28015

§ 371 Date: Apr. 30, 2009

Related U.S. Application Data
(60) Provisional application No. 60/700,645, filed on Jul. 19, 2005.

(51) Int. Cl. A61K 39/07; C07K 2/00; C07K 14/32; A61P 37/02

(52) U.S. Cl. 424/190.1; 530/300; 530/350

(57) ABSTRACT

Compositions and methods for treating a Bacillus anthracis infection in a subject in need thereof are provided.

### CROSS-REFERENCE TO RELATED APPLICATIONS/PATENTS & INCORPORATION BY REFERENCE

This application claims the benefit of U.S. Provisional Application No. 60/700,645, filed Jul. 19, 2005, the entire contents of which are expressly incorporated herein by reference.

Each of the applications and patents cited in this text, as well as each document or reference cited in each of the applications and patents (including during the prosecution of each issued patent; “application cited documents”), and each of the PCT and foreign applications or patents corresponding to and/or paragraphing priority from any of these applications and patents, and each of the documents cited or referenced in each of the application cited documents, are hereby expressly incorporated herein by reference. More generally, documents or references are cited in this text, either in a Reference List before the paragraphs, or in the text itself; and, each of these documents or references (“herein-cited references”), as well as each document or reference cited in each of the herein-cited references (including any manufacturer's specifications, instructions, etc.), is hereby expressly incorporated herein by reference.

### STATEMENT OF POTENTIAL GOVERNMENT INTEREST

The United States government may have certain rights in this invention by virtue of grant number R21 AI055968-01 from the National Institutes of Health.

### BACKGROUND OF THE INVENTION

Bacillus anthracis is a facultative anaerobic, non-motile, gram positive, endospore-forming bacillus, which primarily causes a fatal disease in herbivores (Mock, M. and A. Fouet. 2001. Anthrax. Annu. Rev. Microbiol. 55:647-671). Human infection is acquired upon exposure to endospores and, depending on the route of infection, the disease may manifest as cutaneous (least dangerous and easily treatable), inhalational (often fatal) or gastrointestinal anthrax (rare) (Leppla, S. H., et al., 2002. J. Clin. Invest. 110:141-144; Mock, M. and A. Fouet. 2001). Irrespective of the route of infection, progression to systemic disease can occur. Endospores phagocytosed by macrophages are transported to the regional lymph nodes where they germinate into vegetative bacilli (Leppla, S. H., et al., 2002; Mock, M. and A. Fouet. 2001), which then multiply in the lymphatic system and disseminate into the blood stream causing massive septicemia. The organism then elaborates virulence factors that cause a variety of systemic effects leading to death of the host (Leppla, S. H., et al., 2002; Mock, M. and A. Fouet. 2001).

Thus far, the pathogenicity of B. anthracis has been attributed to the production of virulence factors encoded on two virulence plasmids, pXO1 and pXO2, which are present in all fully virulent strains. pXO2 encodes an antiphagocytic, γ-D-glutamic acid capsule. pXO1 encodes three virulence proteins, protective antigen (PA), lethal factor (LF) and the edema factor (EF), which assemble to form two binary toxins. PA, the non-toxic, receptor-binding moiety can assemble with either EF to form edema toxin (ET), or with LF to form lethal toxin (LT). The enzymatic moiety of ET is an adenylate cyclase (Mock, M. and A. Fouet. 2001) that acts by increasing intracellular levels of cAMP, which is responsible for the edema typical in patients with cutaneous anthrax. The enzymatic moiety of LT is a zinc metalloprotease (Mock, M. and A. Fouet. 2001) that exerts its effect by cleaving mitogen-activated protein kinase kinase (MAPKK). The precise mechanism by which LT causes death in systemic anthrax is still under investigation. Results of recent studies in mice implicate hypoxia-induced tissue injury (Moayeri, M., et al., 2003. J. Clin. Invest. 112:670-682) and genetic factors (Moayeri, M., et al., 2004. Infect. Immun. 72:4439-4447) in LT-mediated lethality, rather than induction of proinflammatory cytokines, as suggested earlier (Mock, M. and A. Fouet. 2001). In addition to the above, results of several recent studies have alluded to other unidentified virulence determinants acting in concert with the aforementioned factors to play a contributory role in anthrax pathogenesis (Brossier, F., et al. 2002. Infect. Immun. 70:661-664; Cohen, S., et al., 2000. Infect. Immun. 68:4549-4558; Little, S. F. and G. B. Knudson, 1986. Infect. Immun. 52:509-512; Pezard, C., M. et al., 1995. Infect. Immun. 63:1369-1372; Stepanov, A. V., et al., 1996. J. Biotechnol 44:155-160; Welkos, S., et al., 2001. Microbiology 147:1677-1685).

Although therapeutic options are available to successfully treat the syndromes of anthrax upon early diagnosis, vaccination may be the most effective strategy to thwart the disease (Leppla, S. H., et al., 2002), especially in target populations likely to be exposed to anthrax spores, such as military personnel and workers in wool and leather industries. Vaccination also remains the most economical means of mass immunization. The anthrax vaccine currently approved for human use in the United States, Anthrax Vaccine Adsorbed (AVA), is a cell-free filtrate prepared from formalin-treated, culture supernatant of a non-proteolytic, toxigenic and unencapsulated, avirulent B. anthracis strain (pXO1⁺, pXO2⁻), V770-NP1-R, adsorbed to the adjuvant, aluminum hydroxide (Joellenbeck, L. M., et al., 2002. National Academy Press, Washington, D.C.). It is administered subcutaneously in a volume of 0.5 ml at 0, 2, and 4 weeks and at 6, 12 and 18 months. Thereafter, boosters administered annually are essential to maintain protective immunity (Friedlander, A. M., et al., 1999. JAMA 282:2104-2106; Leppla, S. H., et al., 2002). A similar vaccine, prepared by adsorbing a sterile culture supernatant-filtrate of the 32F₂ Sterne strain to potassium aluminum sulfate is licensed for use in the United Kingdom (Leppla, S. H., et al., 2002; Whiting, G. C., et al., 2004. Vaccine 22:4245-4251). Studies have demonstrated that AVA is safe (Joellenbeck, L. M., et al., 2002) and protects against both cutaneous (Joellenbeck, L. M., et al., 2002; Leppla, S. H., et al., 2002) and inhalational anthrax (Friedlander, A. M., et al., 1999. JAMA 282:2104-2106; Joellenbeck, L. M., et al, 2002; Leppla, S. H., et al., 2002).

Despite documentation attesting to safety and efficacy of AVA, currently approved human-use anthrax vaccines have several limitations. Immunization with human-use acellular, PA-based vaccines reportedly induces low and transient immune responses (Hambleton, P., et al., 1984. Vaccine 2:125-132; Lincoln, R. and D C Fish. 1970. Anthrax toxin, p. 361-414; T. C. Monte, et al., Academic Press, Inc., New York), and, consistent with this observation, multiple administrations of AVA are required for induction of protective immunity (Brachman, P. S., et al., 1962. Am. J. Public Health 52:632-645). Immunization is associated with local and sometimes systemic reactogenicity attributable to residual LF and EF, which may combine with PA to form active LT and ET, the adjuvant used, and also to the presence of uncharacterized components in vaccine preparations (Joellenbeck, L. M., et al., 2002; Turnbull, P. C. 1991. Vaccine 9:533-539; Whiting, G. C., et al., 2004. Vaccine 22:4245-4251). An additional limitation of AVA includes the lack of standardization in the manufacturing process resulting in batch to batch variations in the amount of PA and the unavailability of reliable assays to measure potency of vaccine preparations (Leppla, S. H., et al., 2002).

Due to the limitations of currently known B. anthracis vaccines, including AVA, there is a need for development of a defined anthrax vaccine free of significant adverse effects and capable of inducing sustained protective immunity.

### SUMMARY OF THE INVENTION

In one embodiment, the invention provides an immunogenic composition comprising at least one anthrax spore-associated protein or immunogenic fragment and/or functional variant thereof.

In another embodiment, the invention provides an immunogenic composition comprising at least one expression vector, wherein the expression vector comprises a nucleic acid molecule encoding an anthrax spore-associated protein or immunogenic fragment and/or functional variant thereof. The expression vector may comprise at least one additional nucleic acid molecule encoding an anthrax spore-associated protein or immunogenic fragment and/or functional variant thereof. Furthermore, the expression vector may be a viral vector or a plasmid vector.

In one embodiment, the immunogenic composition of the invention further comprises protective antigen (PA) (or immunogenic fragment and/or functional variant thereof) or a nucleic acid molecule encoding the PA or a immunogenic fragment and/or functional variant thereof.

In one embodiment, the immunogenic composition of the invention is acellular.

In another embodiment, the immunogenic composition of the invention induces an immunological response in a subject against Bacillus anthracis. The immunological response induced in the subject may be against Bacillus anthracis in the spore form and/or in the bacillus form. The subject may be a mammal. The mammal may be a human.

In one embodiment, the immunogenic composition of the invention further comprises a pharmaceutically acceptable excipient. In another embodiment, the immunogenic composition of the invention further comprises an adjuvant.

In one embodiment, the invention provides an immunogenic composition comprising at least one anthrax spore-associated protein having an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, and immunogenic fragments and/or functional variants thereof.

In another embodiment, the invention provides an immunogenic composition comprising at least one expression vector, wherein the expression vector contains a nucleic acid molecule encoding an anthrax spore-associated protein or immunogenic fragment and/or functional variant thereof, having a nucleic acid sequence is selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, and fragments thereof.

In one embodiment, the invention provides a method for inducing an immunological response in a subject comprising administering to said subject an immunogenic composition comprising at least one anthrax spore-associated protein (or immunogenic fragment and/or functional variant thereof) or an immunogenic composition comprising at least one expression vector, wherein the expression vector comprises a nucleic acid molecule encoding an anthrax spore-associated protein or immunogenic fragment and/or functional variant thereof. The subject may be uninfected with Bacillus anthracis. The subject may be a mammal. The mammal may be a human.

In another embodiment of the method of the invention, the immunogenic composition comprises PA (or an immunogenic fragment and/or functional variant thereof) or a nucleic acid molecule encoding PA (or an immunogenic fragment and/or functional variant thereof). In one embodiment, the subject is uninfected with Bacillus anthracis. In another embodiment, the subject is infected with Bacillus anthracis. In one embodiment of the method of the invention, the administering occurs about one to about sixty days after infection, when the Bacillus anthracis spores have not yet germinated. If the spores have germinated, the administering may be effected in concert with an additional therapy against Bacillus anthracis infection. In one embodiment, the additional therapy comprises antibiotic therapy.

In one embodiment of the method of the invention, the immunological response is against Bacillus anthracis. Bacillus anthracis may exist in the spore form (i.e., in the form of a spore formed by the bacteria) and/or in the bacillus form (i.e., upon activation (germination) of the spore; in this form, the bacteria can reproduce). Accordingly, the immunological response may be against Bacillus anthracis in the spore form and/or the bacillus form.

In another embodiment of the method of the invention, the amount of immunological response is effective to confer substantial protective immunity against infection with Bacillus anthracis in the subject.

In yet another embodiment of the method of the invention, the immunogenic composition is administered 1 to 2 times.

Methods of the invention can further comprise the step of obtaining the anthrax spore-associated protein (or an immunogenic fragment and/or functional variant thereof).

In one embodiment, the invention provides a kit comprising an immunogenic composition comprising at least one anthrax spore-associated protein (or an immunogenic fragment and/or functional variant thereof) or an immunogenic composition comprising at least one expression vector, wherein the expression vector comprises a nucleic acid molecule encoding an anthrax spore-associated protein or immunogenic fragment and/or functional variant thereof and optionally instructions for administering the immunogenic composition to induce an immunological response in a subject and optionally a device and/or vessel for the administration of the composition.

Other aspects of the invention are described in or are obvious from the following disclosure, and are within the ambit of the invention.

### BRIEF DESCRIPTION OF THE FIGURES

The following Detailed Description, given by way of example, but not intended to limit the invention to specific embodiments described, may be understood in conjunction with the accompanying drawings, in which:

FIG. 1 depicts the results of a colony immunoblot assay of the reactivity of pooled, pre-immune, and immune sera with a test clone consisting of E. coli BL21 (DE3)(pSMR-PA) expressing full-length PA, and a negative control comprising of the expression host strain E. coli BL21 (DE3) carrying the native plasmid, pET30a.

### DETAILED DESCRIPTION OF THE INVENTION

### I. Definitions

Unless defined otherwise, all technical and scientific terms used herein have the meaning commonly understood by one of ordinary skill in the art to which this invention belongs. The following references provide one of skill in the art to which this invention pertains with a general definition of many of the terms used in this invention: Singleton et al., Dictionary of Microbiology and Molecular Biology (2d ed. 1994); The Cambridge Dictionary of Science and Technology (Walker ed., 1988); Hale & Marham, The Harper Collins Dictionary of Biology (1991); and Lackie et al., The Dictionary of Cell & Molecular Biology (3d ed. 1999); and Cellular and Molecular Immunology, Eds. Abbas, Lichtman and Pober, 2^{nd} Edition, W.B. Saunders Company. For the purposes of the present invention, the following terms are further defined.

The term “anthrax vaccine” refers to a vaccine administered in any known form, such as, for example, a protein antigen, such as a spore protein, or a nucleic acid encoding the spore protein, or some combination thereof, that is specifically immunoreactive against Bacillus anthracis, the causative agent of anthrax, wherein an immune response is generated against the vaccine which in turn immunizes the subject against infection by B. anthracis. Alternatively, or at the same time, the anthrax vaccine can also refer to a vaccine composition that elicits an immune response against anthrax toxins, such as, for example, protective antigen.

The term “anthrax spore-associated protein” refers to any protein obtained or derived from the spore (e.g., interior or exterior) or spore form of a Bacillus anthracis isolate strain or the like.

The phrase “specifically immunoreactive” can refer to a binding reaction between an antibody and a protein, compound, or antigen, having an epitope recognized by the antigen binding site of the antibody. This binding reaction is determinative of the presence of a protein, antigen or epitope having the recognized epitope amongst the presence of a heterogeneous population of proteins and other biologics. Thus, under designated immunoassay conditions, the specified antibodies can bind to a protein having the recognized epitope and bind, if at all, to a detectably lesser degree to other proteins lacking the epitope which are present in the sample. An antibody that is specifically immunoreactive with an antigen can bind to that antigen and form a complex therewith. In an in vivo context, “specifically immunoreactive” can refer to the conditions under which in an animal forms an immune response against a vaccine or antigen, e.g. a humoral response to the antigen (the production of antibodies, against a vaccine, protein, compound, or antigen presented thereto under immunologically reactive conditions) or a cell-mediated (also herein as “cellular immune response”, i.e. a response mediated by T lymphocytes against the vaccine, protein, compound or antigen presented thereto).

The term “immunity” can refer to both “natural” (native or innate) immunity or “acquired” (specific) immunity. Natural immunity relates to a collection of innate mechanisms in a subject that are capable of warding off or protecting against infection by a foreign organism, virus or substance, such as, physical barriers, phagocytic cells and eosinophils in the blood and tissues, natural killer cells, and various blood-borne molecules (e.g. complement system) that are already present in a subject prior to infection by the invading foreign organism, virus or substance. Acquired or specific immunity refers to immunity to a foreign organism, virus or substance (i.e. the antigen) that is induced by the presence of the invading organism, virus, or substance which encompasses both humoral and cell-mediated mechanisms.

An “immunogenic composition” is an antigenic preparation of the invention, including, e.g., a protein or immunogenic fragment thereof or a polynucleotide encoding a protein or immunogenic fragment thereof or a polysaccharide, a combination of more than one protein or immunogenic fragment thereof, or a combination of a protein (or immunogenic fragment thereof) and a polynucleotide encoding a protein (or immunogenic fragment thereof) administered to stimulate the recipient's humoral and cellular immune systems to one or more of the antigens present in the vaccine preparation. The term “immunogenic composition” includes the terms vaccine and immunological composition. “Vaccination” or “immunization” is the process of administering an immunogenic composition and stimulating an immune response to an antigen.

An “antigen” or “immunogen” is any agent, e.g., a polynucleotide, a protein, a peptide, or a polysaccharide, that elicits an immune response and is therefore characterized as “immunogenic.” The antigen can be attached to an invading organism or virus, e.g. a cell surface protein or viral capsule protein, or unattached, e.g. a circulating anthrax toxin.

An “immune response” refers to the activities of the immune system in response to an invading antigen, organism, virus, or substance, including mechanisms relating to natural and acquired immunity, and humoral and cell-mediated immunity, including especially the induction of antigen-specific antibodies and the activation and proliferation of specific cytotoxic T-cells after contact with an antigen, organism, virus or substance.

The term “antibody” refers to the family of glycoproteins encoded by an immunoglobulin gene(s) produced in connection with a humoral immune response which specifically recognize and bind to antigens to which they are raised. In the body, antibodies can be produced in a membrane-bound form by B lymphocytes as well as in a secreted form by progeny of B cells that differentiate in response to antigenic stimulation. The term “antibody” can further refer broadly to any immunologic binding agent such as IgG, IgM, IgA, IgD and IgE. The term “antibody” is also used to refer to any antibody-like molecule that has an antigen binding region, and includes antibody fragments such as Fab′, Fab, F(ab′)₂, single domain antibodies (DABs), Fv, scFv (single chain Fv), and engineering multivalent antibody fragments such as dibodies, tribodies and multibodies. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

The anthrax “protective antigen” (PA) is an 83 kDa protein (SEQ ID NO:159) produced by Bacillus anthracis. PA is one of two protein components of the lethal or anthrax toxin produced by B. anthracis. The 83 kDa PA binds at its carboxyl-terminus to a cell surface receptor, where it is specifically cleaved by a protease, e.g., furin, clostripain, or trypsin. This enzymatic cleavage releases a 20 kDa amino-terminal PA fragment, while a 63 kDa carboxyl-terminal PA fragment remains bound to the cell surface receptor. The description of protective antigen includes binary toxin functional equivalents such as protein Ib of C. perfringens.

“Parenteral” administration of a vaccine includes, e.g., subcutaneous, intravenous, intramuscular, or intrasternal injection or infusion techniques.

“Antigen presenting cells” are cells, e.g., dendritic cells or macrophages, that process peptide antigens through the MHC class I processing pathway so that the antigen-MHC class I complex is displayed on their cell surface. A “dendritic” cell is a motile, non-phagocytic adherent cell that acts as an efficient antigen-presenting cell and moves readily between the lymph nodes and other organs. Dendritic cells are further classified into subgroups, including, e.g., follicular dendritic cells, Lagerhans dendritic cells, and epidermal dendritic cells.

“Anthrax toxin” is a binary toxin produced by B. anthracis, composed of LF and PA. Anthrax toxin may also refer to the binary edema toxin of B. anthracis, composed of LF and EF (edema factor). A “binary toxin” is a bacterial toxin that is composed of two separate proteins that associate to form the toxin.

“Substantial protective immunity” refers to a state in which the subject's body responds specifically to the antigen(s), and a protective response is mounted against the pathogenic agent (in this case, Bacillus anthracis), said response comprising an alteration in the reactivity of the subject's immune system in response to the antigen(s), potentially involving antibody production, induction of cell-mediated immunity, and/or complement activation. The response results in a degree of protection (i.e., a protective immune response) comprising protection from Bacillus anthracis infection, or further infection or spread of infection if the subject is already infected with Bacillus anthracis.

An “expression vector” is a vector used for transfer of genetic information (in the form of a nucleotide sequence) into a cell, where a recombinant protein encoded by said genetic information can then be expressed.

The term “obtaining” as in “obtaining the spore associated protein” is intended to include purchasing, synthesizing or otherwise acquiring the spore associated protein (or indicated substance or material).

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### II. Compositions and Methods of the Invention

Peptide-Based Immunogenic Compositions

In one aspect, the present invention is directed to immunogenic compositions comprising at least one antigen that is capable of eliciting an immune response and of providing a protective effect against B. anthracis or a toxin thereof.

One embodiment provides an immunogenic composition of the invention that comprises at least one anthrax spore-associated protein or a variant form thereof or an immunogenic fragment thereof. As used herein, the term “immunogenic fragment thereof” can refer to a peptide which is at least 6 amino acids in length, preferably at least about 15 amino acids in length, and has the ability to elicit production of antibodies that bind to the wild-type protein from which it is derived, and the ability to elicit an immune response and protective effect that is the same or substantially the same as the immune response and protective effect elicited by the native protein from which it is derived.

It will be appreciated by the person of skill in the art to which the present invention pertains that there are numerous possible ways to determine whether a particular antigen fragment of the invention is an “immunogenic fragment” of the antigens of the invention (e.g. anthrax spore-associated proteins or anthrax PA). The invention encompasses any method for measuring, evaluating or determining whether an antigen fragment is immunogenic, including, for example, in vitro or in vivo testing. For example, in in vitro methods, an immunogenic antigen fragment of interest can be tested using antibody-binding assays, e.g. immunoassays, that compare the strength of antibody binding to the native antigen and the immunogenic antigen fragment of interest. A detailed review of immunological assay design, theory and protocols can be found in numerous texts in the art, including “Practical Immunology”, Butt, W. R., ed., (1984) Marcel Dekker, New York and “Antibodies, A Laboratory Approach”, Harlow et al. eds. (1988) Cold Spring Harbor Laboratory. In in vivo methods, an immunogenic antigen fragment of interest can be tested in an animal, such as a mouse or rabbit or cow, to determine if the animal produces antibodies raised against the antigen fragment of interest that are capable eliciting or establishing a protective response or alternatively, if the antibodies formed against the immunogenic antigen fragment of interest specifically react with the native antigen from which the antigen fragment is derived.

Antigen fragments that are similarly immunogenic or substantially immunogenic as the native antigens of the invention, e.g. the anthrax spore-associated proteins of the invention or anthrax PA, can be prepared in any suitable manner available to one of ordinary skill in the art. Such methods can include genetic engineering methods, whereby a nucleic acid molecule encoding only a partial amino acid sequence (i.e. antigen fragment) of the native antigen is prepared and used to either express the antigen fragment or is used to administer to a subject for achieving in vivo expression of the antigen fragment. Physical and/or chemical and/or enzymatic methods can also be used to prepare the immunogenic fragments of the invention, including, for example, peptidase treatment or chemical cleavage. Methods for producing immunogenic fragments of the inventive anthrax spore-associated proteins and PA by way of physical and/or chemical and/or enzymatic methods can be found in the technical literature, for example, in Methods in Enzymology, Volume 182, Guide to Protein Purification, Eds. J. Abelson, M. Simon, Academic Press, 1^{st} Edition, 1990. In addition, immunogenic antigen fragments of the invention can be synthesized using known and available methods and techniques for protein/peptide synthesis, for example, as described in Chemical Approaches to the Synthesis of Peptides and Proteins (Hardcover), Eds. P. Lloyd-Williams, F. Albericio, and E. Giralt, CRC Press, 1^{st} Edition, 1997.

In certain embodiments, the anthrax spore-associated protein comprises an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, and immunogenic fragments or functional variants thereof.

The anthrax spore-associated proteins of the present invention can be a full-length, wild-type, mature anthrax spore-associated protein, i.e. “native protein.” The term “anthrax spore-associated protein”, as used herein, also can encompass naturally-occurring and man-made variant anthrax spore-associated proteins whose amino acid and/or nucleotide sequences differ from the sequences shown herein. Such variant proteins can have an amino acid sequence which is at least 90% identical, preferably at least 95% identical, or more preferably at least 99% identical to the specific amino acid sequences shown herein. Such variant proteins can have an altered sequence in which one or more of the amino acids in the specific anthrax spore-associated protein sequence is substituted, or in which one or more amino acids are deleted from or added to such sequence. Such variants include degenerate variants. Such variants, when injected into an animal, elicit production of antibodies that bind to the mature, wild-type anthrax spore-associated protein in question, i.e., the anthrax spore-associated protein whose sequence corresponds to one of those depicted herein.

The term “variant form thereof,” or equivalently “functional variant thereof” as used herein, can refer to a distinct but related version of the at least one anthrax spore-associated protein or other proteins of the invention (e.g. the B. anthracis PA) that can differ with respect to the amino acid sequence of the variant as compared to the native protein, the underlying nucleotide sequence encoding the variant as compared to the native nucleotide sequence, or the state of chemical modification of the variant as compared to the native protein, e.g. glycosylation pattern. The functional variant forms of the antigens of the invention include both those that are created by man, e.g. chemical modification or genetic engineering, or those that are produced in nature, e.g. by naturally occurring genetic mutation. The functional variants of the invention can differ from the native antigens as a result of conservative/degenerate nucleotide and/or amino acid sequence substitutions. Preferably, the functional variants of the invention will contain at least 90% sequence identity, more preferably at least 95% sequence identity, and still more preferably, at least 99% sequence identity with the native proteins of the invention, e.g. the anthrax spore-associated proteins and/or the anthrax PA. Functional variants of the invention are functionally equivalent to the individual native antigens from which they derive or are otherwise obtained.

As used herein, the terms percent (%) sequence identity or percent (%) homology are used synonymously as a measure of the similarity of two or more amino acid sequences. Methods for determining percent (%) sequence identity or percent (%) homology are well known in the art.

For the purposes of the present invention, percent (%) sequence identity or homology can be determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448. Advantageous for use according to the present invention is the WU-BLAST (Washington University BLAST) version 2.0 software. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein).

In general, comparison of amino acid sequences is accomplished by aligning an amino acid sequence of a polypeptide of a known structure with the amino acid sequence of a the polypeptide of unknown structure. Amino acids in the sequences are then compared and groups of amino acids that are homologous are grouped together. This method detects conserved regions of the polypeptides and accounts for amino acid insertions and deletions. Homology between amino acid sequences can be determined by using commercially available algorithms (see also the description of homology above). In addition to those otherwise mentioned herein, mention is made too of the programs BLAST, gapped BLAST, BLASTN, BLASTP, and PSI-BLAST, provided by the National Center for Biotechnology Information. These programs are widely used in the art for this purpose and can align homologous regions of two amino acid sequences.

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Alternatively or additionally, the term “homology” or “identity”, for instance, with respect to a nucleotide or amino acid sequence, can indicate a quantitative measure of homology between two sequences. The percent sequence homology can be calculated as (N_{ref}−N_{dif})*100/−N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{ref}=8; N_{dif}=2).

Alternatively or additionally, “homology” or “identity” with respect to sequences can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur & Lipman, Proc Natl Acad Sci USA 1983; 80:726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being-considered equal to uracil (U) in RNA sequences.

And, without undue experimentation, the skilled artisan can consult with many other programs or references for determining percent homology.

In one embodiment of the invention, the substitutions of the functional variants of the inventive antigens are conservative amino acid substitutions, in which the substituted amino acid has similar structural or chemical properties with the corresponding amino acid in the reference sequence. By way of example, conservative amino acid substitutions involve substitution of one aliphatic or hydrophobic amino acid, e.g. alanine, valine, leucine and isoleucine, with another; substitution of one hydroxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic residue, e.g. phenylalanine and tyrosine, with another; replacement of one basic residue, e.g. lysine, arginine and histidine, with another; and replacement of one small amino acid, e.g., alanine, serine, threonine, methionine, and glycine, with another.

By way of example, functional variant sequences, which are at least 90% identical, have no more than 1 alteration, i.e., any combination of deletions, additions or substitutions, per 10 amino acids of the flanking amino acid sequence. Percent identity is determined by comparing the amino acid sequence of the variant with the reference sequence using MEGALIGN module in the DNA STAR program.

The term “anthrax spore-associated protein”, as used herein, can sometimes encompass functional variants and immunogenic antigen fragments that are encoded by polynucleotide variants, which are polynucleotides that differ from a reference polynucleotide. Generally, the differences are limited so that the nucleotide sequences of the reference and the variant are closely similar overall and, in many regions, identical. The present invention encompasses both allelic variants and degenerate variants.

As iterated briefly above, a variant of a polynucleotide may be a naturally occurring variant such as a naturally occurring allelic variant, or it may be a variant that is not known to occur naturally. By an “allelic variant” is intended one of several alternate forms of a gene occupying a given locus on a chromosome of an organism (Lewin, (1989), PNAS 86:9832-8935). Diploid organisms may be homozygous or heterozygous for an allelic form. Non-naturally occurring variants of the polynucleotide may be made by art-known mutagenesis techniques, including those applied to polynucleotides, cells or organisms.

Polynucleotide variants referred to as “degenerate variants” constitute polynucleotides which comprise a sequence substantially different from those described herein but which, due to the degeneracy of the genetic code, still encode a polypeptide comprised in an immunogenic composition of the present invention. That is, all possible polynucleotide sequences that encode the polypeptides defined herein as potentially comprised in an immunogenic composition of the present invention are contemplated. This includes the genetic code and species-specific codon preferences known in the art.

Nucleotide changes present in a variant polynucleotide may be silent, which means that they do not alter the amino acids encoded by the polynucleotide. However, nucleotide changes may also result in amino acid substitutions, additions, deletions, fusions and truncations in the polypeptide encoded by the reference sequence. The substitutions, deletions or additions may involve one or more nucleotides. The variants may be altered in coding or non-coding regions or both. Alterations in the coding regions may produce conservative or non-conservative amino acid substitutions, deletions or additions. In one embodiment of the present invention, the polynucleotide variants encode polypeptides which retain substantially the same biological properties or activities as the proteins identified herein.

In another embodiment, the peptide-based immunogenic composition of the invention comprises an anthrax spore-associated protein or an immunogenic fragment thereof and the B. anthracis PA protein or an immunogenic fragment thereof. The full-length, wild-type PA protein has a molecular weight of 83 kDA and comprises 735 amino acids. The full-length, wild-type, mature PA protein comprises the amino acid sequence, SEQ ID NO:160, shown herein. The term “PA protein”, as used herein, can also encompass wild-type and mutated PA proteins whose sequence differs slightly from the afore-mentioned sequence. Such variants have an amino acid sequence which is at least 90% identical, preferably at least 95% identical, more preferably at least 99% identical to the amino acid sequence in question. Suitable variants elicit production of antibodies that bind to the wild-type PA protein.

In one embodiment, the anthrax spore-associated protein and optional PA components of the immunogenic compositions of the invention are pure, meaning that the polypeptides have been isolated and purified to substantial homogeneity. A polypeptide that produces a single peak that is at least 95% of the input material on an HPLC column is considered “pure” for the purposes of this invention. Utilizing proteins of high purity may signify the absence of adjuvant materials such as alum, as well as the elimination of common contaminants or additives used in prior art anthrax vaccines.

Any known method of purification that is suitable for producing pure anthrax spore-associated protein or PA polypeptides or the immunogenic and/or functional variants thereof, may be used, for example, using chromatography, and can be found described in the technical literature, for example, in Methods in Enzymology, Volume 182, Guide to Protein Purification, Eds. J. Abelson, M. Simon, Academic Press, 1^{st} Edition, 1990. Thus, suitable materials for performing such purification steps, such as chromatographic steps, are known to those skilled in the art.

In one embodiment of the present invention, the peptide-based immunogenic composition of the invention can be delivered to a subject in need thereof employing an attenuated bacterial vaccine vector, such as that described in U.S. Pat. No. 6,383,496, which is incorporated herein in its entirety by reference. Such vectors include, without limitation, attenuated strains of Vibrio cholerae, Salmonella typhimurium, Listeria monocytogenes, and lactococcal species. Attenuated bacterial vaccine vectors, such as those above, can effectively deliver proteins to the mucosal immune system, consequently engendering a protective mucosal immune response in the subject. Such vaccines and “carrier microbes” can serve as vehicles for delivering desired gene products such as the antigens of the invention, the immunogenic fragments thereof and functional variants thereof also of the invention, to subjects, including humans, as well as for delivering nucleic acids, either DNA or RNA, to target cells, such as human cells.

The attenuated microbes, i.e. attenuated bacterial vaccine vectors of the present invention, contain at least one recombinant gene capable of expressing a desired gene product, e.g. the antigens of the invention (and immunogenic fragments and functional variants thereof), which allows their use as carriers or delivery vehicles of the gene product to subjects, including humans. By delivery of the desired gene product it is meant that either the gene product or the polynucleotide, i.e. nucleic acid, either DNA or RNA, encoding the product is delivered to the subject.

### Nucleic Acid-Based Immunogenic Composition

Another aspect of the invention is directed to an immunogenic composition comprising at least one expression vector comprising a nucleic acid molecule that encodes an antigen of the invention, e.g. an anthrax spore-associated protein, or immunogenic fragment thereof, or functional variant thereof, which are capable of eliciting an immune response and a protective effect against B. anthracis or toxins thereof.

It is generally known that the mammalian system reacts to invading pathogens by mounting two broad defenses: the cell-mediated response and the humoral response. Viral and other intracellular infections are controlled primarily by the cell-mediated immune system. This control is achieved through recognition of foreign antigen displayed on the cell surface of an infected cell.

The cell-mediated immune system responds to endogenous antigen presented by the MHC class I processing pathway. Without being bound by theory, an objective for a vaccine that stimulates the cell-mediated immune system is to deliver protein antigen to the cell cytosol for processing and subsequent presentation by MHC class I molecules.

The use of deoxyribonucleic acid (DNA) molecules for vaccination has been known since the beginning of the 1990s (e.g. Wolf et al. Science 1990. 247. 1465-1468). This vaccination technique induces cellular and humoral immunity after in vivo transfection of cells of the subject to be vaccinated with DNA or RNA molecules encoding immunologically active proteins.

It will be appreciated that the use of DNA molecules for vaccination contrasts with “traditional” vaccination techniques which involve the introduction into an animal system of an antigen which can induce an immune response in the animal, and thereby protect the animal against infection. Following the observation in the early 1990's that plasmid DNA could directly transfect animal cells in vivo, significant research efforts have been undertaken to develop vaccination techniques based upon the use of DNA plasmids (and other deliverable forms of DNA molecules) to induce immune responses, by direct introduction into animals DNA which encodes for antigenic peptides. Such techniques, which are referred to as “DNA immunization” or “DNA vaccination” have now been used to elicit protective antibody (humoral) and cell-mediated (cellular) immune responses in a wide variety of pre-clinical models for viral, bacterial and parasitic diseases. Such techniques are contemplated by the present invention.

DNA vaccines can consist of a bacterial plasmid vector into which is inserted a viral promoter, a gene of interest which encodes for an antigenic peptide and a polyadenylation/transcriptional termination sequence. The gene of interest may encode a full protein (e.g. anthrax spore-associated protein of the invention) or simply an antigenic peptide (e.g. immunogenic fragment thereof) relating to a pathogen or toxin of interest which is intended to be protected against. The plasmid can be grown in bacteria, such as for example E. coli and then isolated and prepared in an appropriate medium, depending upon the intended route of administration, before being administered to the host. Following administration, the plasmid is taken up by cells of the host wherein the encoded peptide is produced. The plasmid vector will preferably be made without an origin of replication which is functional in eukaryotic cells, in order to prevent plasmid replication in the mammalian host and integration within chromosomal DNA of the animal concerned.

DNA vaccination can be advantageous over traditional forms of vaccination in several respects. Firstly, it is predicted that because the proteins which are encoded by the DNA sequence are synthesized in the host, the structure or conformation of the protein will be similar to the native protein associated with the disease state. It is also likely that DNA vaccination can offer protection against different strains of a virus, by generating cytotoxic T lymphocyte responses that recognize epitopes from conserved proteins. Furthermore, because the plasmids are taken up by the host cells where antigenic protein can be produced, a long-lasting immune response can be elicited. The technology also offers the possibility of combining diverse immunogens into a single preparation to facilitate simultaneous immunization in relation to a number of disease states.

Further background on DNA vaccination can be found in Donnelly J. et al, “DNA Vaccines” Annu. Rev. Immunol. 1997, 15: 617 48, the disclosure of which is included herein in its entirety by way of reference.

Accordingly, in one embodiment, the invention provides a DNA immunogenic composition, i.e. a DNA vaccine composition, comprising at least one expression vector, which may be expressed by the cellular machinery of the subject to be vaccinated or inoculated, and, optionally, a pharmaceutically acceptable excipient. The nucleotide sequence of this plasmid can encode, inter alia, one or more anthrax spore-associated immunogens (proteins) capable of inducing, in the subject to be vaccinated or inoculated, a cellular immune response (mobilization of the T lymphocytes) and/or a humoral immune response (stimulation of the production of antibodies specifically directed against the immunogen). The encoded immunogens can also be immunogenic fragments or functional variants of the anthrax spore-associated proteins as described herein. Nucleic acid-based immunogenic compositions, i.e. DNA vaccines, are described for example, in U.S. Pat. Nos. 5,589,466 and 7,074,770, the disclosures of which are hereby incorporated by reference in their entireties.

In another embodiment, the present invention provides a pharmaceutical and/or immunogenic polypeptide to the interior of a cell of a vertebrate in vivo, and a method for delivering the pharmaceutical and/or immunogenic polypeptide comprising the step of introducing a preparation comprising a pharmaceutically acceptable injectable carrier and a naked polynucleotide operatively coding for the polypeptide (e.g. anthrax spore-associated protein or immunogenic or functional variant thereof) into the interstitial space of a tissue comprising the cell, whereby the naked polynucleotide is taken up into the interior of the cell and has an immunogenic effect on the vertebrate, thereby immunizing the vertebrate against infection by B. anthracis or a toxin thereof.

The anthrax spore-associated protein polynucleotides of the various embodiments of the invention can comprise a nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, and fragments thereof, shown herein.

In another aspect, the present invention is directed to immunogenic compositions comprising an anthrax spore-associated protein polynucleotide and a polynucleotide which encodes the B. anthracis PA (protective antigen) protein, referred to hereinafter as the “PA polynucleotide”, or an immunogenic fragment or functional variant thereof, referred to hereinafter as the “PA fragment polynucleotide”. The PA polynucleotide may encode a full-length mature PA protein or, alternatively, a full-length, immature PA protein which comprises a nucleotide sequence encoding a signal sequence. In one embodiment, the PA polynucleotide comprises the nucleotide sequence, SEQ ID NO:159, shown herein. The anthrax spore-associated protein and B. anthracis PA protein may, in another aspect, both be encoded by one nucleic acid sequence.

The polynucleotide may be either a DNA or RNA sequence. All forms of DNA, whether replicating or non-replicating, which do not become integrated into the genome, and which are expressible, are within the methods contemplated by the invention. When the polynucleotide is DNA, it can also be a DNA sequence which is itself non-replicating, but is inserted into a plasmid, and the plasmid further comprises a replicator (e.g. an origin of replication). The DNA may be a sequence engineered so as not to integrate into the host cell genome. The polynucleotide sequences may code for a polypeptide which is either contained within the cells or secreted therefrom, or may comprise a sequence which directs the secretion of the peptide. With the availability of automated nucleic acid synthesis equipment, both DNA and RNA can be synthesized directly when the nucleotide sequence is known or by methods which employ PCR cloning.

The anthrax spore-associated protein polynucleotide, anthrax spore-associated protein fragment polynucleotide PA polynucleotide, and PA fragment polynucleotides can be incorporated into the immunogenic compositions in one of several forms, including a linear molecule, a plasmid, a viral construct, or a bacterial construct, such as, for example, a Salmonella construct to provide a vaccine. In those cases where the immune response is elicited by administration of both the anthrax spore-associated protein polynucleotide or anthrax spore-associated protein fragment polynucleotide and the PA polynucleotide or PA fragment polynucleotide, the polynucleotides may be incorporated into separate nucleic acid molecules which are co-administered to the subject. Alternatively, the anthrax spore-associated protein polynucleotide (or anthrax spore-associated protein fragment polynucleotide) and PA polynucleotide (or PA fragment polynucleotide) may be incorporated into the same nucleic acid. In such case, the anthrax spore-associated protein polynucleotide and PA polynucleotide may be operably linked to separate promoters or to the same promoter.

In addition, the present invention contemplates pharmaceutical compositions that comprise a combination of polypeptides and polynucleotides wherein the polynucleotides can encode the polypeptides of the invention. For example, one pharmaceutical composition of the invention can comprise both a B. anthracis spore-associated polypeptide (or immunogenic or functional variant thereof) and a polynucleotide encoding B. anthracis PA (or immunogenic or functional variant thereof). Alternatively, the pharmaceutical composition can comprise at least one B. anthracis spore-associated polypeptide (or immunogenic or functional variant thereof) and a polynucleotide encoding at least one B. anthracis spore-associated protein (or immunogenic or functional variant thereof). In such pharmaceutical compositions, the polypeptide component and polypeptide component can be contained together in the same composition or each can be separately contained and provided as separate components which can be co-administered. For the purposes of this invention, “co-administering” is administration of two or more medicaments or pharmaceutical compositions (e.g. a polypeptide component and a polynucleotide component) at the same time or at about the same time, e.g. sequential administration. Sequential administration also encompasses an administration regimen occurring in some pattern over the course of days, weeks, or months, such as, for example, administering on a first day a polypeptide component followed by on a second day a polynucleotide component. There is no intended limitation on the manner in which co-administration may occur and the skilled artisan will be able to competently design a suitable co-administration regimen.

In an additional embodiment of the invention, certain modifications in the anthrax spore-associated antigens (proteins) exist, due to, for example, deletions of part of the nucleotide sequence encoding the antigen, insertions of a DNA fragment into the nucleotide sequence encoding the antigen, or into non-translated regions upstream or downstream. Such modifications may enhance the efficacy of the DNA immunogenic compositions, for example, by enhancing the level of expression of the antigen or its presentation. However, care must be taken that manipulations of the nucleotide sequence encoding the antigen do not bring about a reduction or loss of the initial immunological activity. Furthermore, the modifications carried out on the nucleotide sequence of one antigen cannot necessarily be directly transposed to another antigen, because antigens do not always have the same structural arrangements.

In one embodiment of the DNA immunogenic compositions of the invention, the expression vector can be a plasmid. The term “plasmid” covers a DNA transcription unit comprising a polynucleotide sequence comprising the sequence of the gene to be expressed and the elements necessary for its expression in vivo. In additional embodiments, the circular plasmid form, supercoiled or otherwise, or the linear form may be employed. When several genes are present in the same plasmid (e.g. the combination of a nucleotide encoding a B. anthracis spore-associated protein and a nucleotide encoding B. anthracis PA), they may be provided in the same transcription unit or in two transcription units or in several different or more transcription units. In another embodiment of the DNA immunogenic composition of the invention, the expression vector is a virus. Viral vectors appropriate for delivery of a polynucleotide sequence are known in the art.

The anthrax spore-associated protein polynucleotide or anthrax spore-associated protein fragment polynucleotide may be operably linked to a promoter which drives expression of the protein or fragment. Such promoter may be a constitutive promoter, such as, for example, the viral promoter derived from cytomegalovirus (CMV). Other viral promoters include, without limitation, CMV-IE, SV40 virus early or late promoter, and the Rous Sarcoma virus LTR promoter. Employable cellular promoters include, without limitation, that of a cytoskeleton gene, such as the desmin promoter, or, alternatively, the actin promoter. Inducible promoters are likewise contemplated, such as, for example, the lac promoter or a tissue specific promoter, such as the whey acidic protein promoter.

In one embodiment of the DNA immunogenic composition of the invention, the nucleotide sequence encoding the immunogen is in an optimized form. Optimization is understood to mean any modification of the nucleotide sequence, in particular which manifests itself at least by a higher level of expression of this nucleotide sequence, and/or by an increase in the stability of the messenger RNA encoding this antigen, and/or by the triggered secretion of this antigen into the extracellular medium, and having as direct or indirect consequence an increase in the immune response induced. Such optimization of the antigen of interest may, for example, consist in the insertion of a stabilizing intron into the gene encoding the antigen of interest to avoid the aberrant splicings of its messenger RNA and maintain the physical integrity of the latter.

In additional embodiments of the DNA immunogenic compositions of the invention, the expression vector also contains a ribosome binding site, including an internal ribosome site, for translation initiation and a transcription terminator. The vector may further include appropriate sequences for amplifying expression. In addition, expression vectors may contain one or more selectable marker genes to provide a phenotypic trait for selection of transformed host cells, such as dihydrofolate reductase or neomycin resistance for eukaryotic cell culture, or such as tetracycline or ampicillin resistance for bacterial cell cultures such as E. coli. One of ordinary skill in the art will appreciate that the particular selectable marker chosen will, like the expression vector itself, depend on the properties of the host organism.

The expression vector containing the appropriate DNA sequence(s) as hereinabove described, as well as an appropriate promoter or expression control sequence, may be employed to transform an appropriate host to permit the host to express the protein. As representative examples of appropriate host cells, there may be mentioned bacterial cells, such as E. coli, Streptomyces, Salmonella typhimurium; fungal cells, such as yeast; insect cells such as Drosophila and Sf9; animal cells such as CHO, COS or Bowes melanoma; plant cells, etc. The selection of an appropriate host for this type of recombinant polypeptide production is also within the capability of those skilled in the art from the teachings herein. Suitable expression vectors and promoters are replicable and viable in the selected host cell. The quantity of DNA used in the vaccines according to the present invention can be between about 10 micrograms and about 2000 micrograms and preferably between about 50 micrograms and about 1000 micrograms. Persons skilled in the art will have the competence necessary to precisely define the effective dose of DNA to be used for each immunization or vaccination protocol.

Introduction of the DNA vaccine vectors of the present invention into the host cell can be effected by any known method, including calcium phosphate transfection, DEAE-Dextran mediated transfection, or electroporation (see Davis et al., Basic Methods in Molecular Biology, (1986)). It is possible for the vectors of the present invention to be administered in a naked form (that is as naked DNA not in association with liposomal formulations, with viral vectors or transfection facilitating proteins) suspended in an appropriate medium, for example a buffered saline solution such as PBS and then injected intramuscularly, subcutaneously, intraperitonally or intravenously, although some earlier data suggests that intramuscular or subcutaneous injection is preferable (Brohm W et al, “Routes of Plasmid DNA Vaccination that Prime Murine Humoral and Cellular Immune Reponses,” Vaccine, Vol 16, No. 9/10, pp 949 954, 1998), (the disclosure of which is incorporated herein in its entirety by way of reference). It is additionally possible for the vectors to be encapsulated by, for example, liposomes or within polylactide co-glycolide (PLG) particles (Vordermeier, H. M., Coombs, A. G. A., Jenkins, P. McGee, J. P., O'Haga, D. T. Davis, S. S, and Singh, M. Synthetic delivery systems for tuberculosis vaccines: immunological evaluation of the M. tuberculosis 38 kDa protein entrapped in biodegradable PLG microparticles. Vaccine 13: 1576 1582 1995) for administration via the oral, nasal or pulmonary routes. It is also possible, according to a preferred embodiment of the invention, for intradermal administration of the vector, preferably via use of gene-gun (particularly particle bombardment) administration techniques. Such techniques may involve lyophilization of a suspension comprising the vector and subsequent coating of the vector on to gold beads which are then administered under high pressure into the epidermis, such as, for example, as described in Haynes J R. McCabe D E. Swain W F. Wedera G. Fuller J T. Particle-mediated nucleic acid immunization. Journal of Biotechnology. 44: 37 42, 1996. The amount of DNA delivered can vary significantly, depending upon the species and weight of mammal being immunized, the nature of the disease state being treated/protected against, the vaccination protocol adopted (i.e. single administration versus repeated doses), the route of administration and the potency and dose of the adjuvant compound chosen. Based upon these variables, a medical or veterinary practitioner will readily be able to determine the appropriate dosage level.

It is possible for the DNA vector, including the DNA sequence encoding the antigenic peptide, to be administered on a once off basis or to be administered repeatedly, for example, between 1 and 7 times, preferably between 1 and 4 times, at intervals between about 1 day and about 18 months. Once again, however, this treatment regime will be significantly varied depending upon the size and species of animal concerned, the disease which is being treated/protected against, the amount of DNA administered, the route of administration, the potency and dose of adjuvant compound selected and other factors which would be apparent to a skilled veterinary or medical practitioner. To enhance the immune response, the DNA vaccine compositions of the present invention can be administered with at least one adjuvant, such as those described in U.S. Pat. No. 7,074,770 which is incorporated by reference herein in its entirety. Any adjuvant compound that serves to increase the immune response induced by the antigen (either directly administered or expressed in a DNA vaccine) is contemplated by the present invention.

Formulations for injection of the DNA vaccines of the invention via, for example, the intramuscular, intraperitonile, or subcutaneous administration routes include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described. Formulations suitable for pulmonary administration via the buccal or nasal cavity are presented such that particles containing the active ingredient, desirably having a diameter in the range of 0.5 to 7 microns, are delivered into the bronchial tree of the recipient. Possibilities for such formulations are that they are in the form of finely comminuted powders which may conveniently be presented either in a pierceable capsule, suitably of, for example, gelatin, for use in an inhalation device, or alternatively, as a self-propelling formulation comprising active ingredient, a suitable liquid propellant and optionally, other ingredients such as surfactant and/or a solid diluent. Self-propelling formulations may also be employed wherein the active ingredient is dispensed in the form of droplets of a solution or suspension. Such self-propelling formulations are analogous to those known in the art and may be prepared by established procedures. They are suitably provided with either a manually-operable or automatically functioning valve having the desired spray characteristics; advantageously the valve is of a metered type delivering a fixed volume, for example, 50 to 100 microliters upon each operation thereof.

### Preparation of Immunogenic Compositions

i) Preparing the Anthrax Spore-Associated Protein, the PA Protein, and Fragments Thereof

The anthrax spore-associated proteins (or immunogenic and functional variants thereof) and PA proteins (or immunogenic and functional variants thereof) can be obtained by any suitable means, including, for example, purification from B. anthracis cultures or prepared as recombinant proteins from cultures of recombinant organisms. Within the context of this application, “purified” anthrax spore-associated proteins and PA proteins (and any immunogenic or function variant thereof) refers to preparations that are comprised of at least 90% anthrax spore-associated protein or PA protein, and no more than 10% of the other proteins found in the cell-free extracts or extracellular medium from which these proteins are isolated. Such preparations are said to be at least 90% pure. The PA protein may be isolated and purified from the supernatant of B. anthracis cultures using techniques known in the art, for example, as described in Methods Enzymol. 165: 103-116, 1988, which is specifically incorporated herein by reference.

In one embodiment, the anthrax spore-associated protein, PA protein, and any immunogenic fragments or functional variants thereof are prepared using recombinant techniques. Such techniques employ nucleic acid molecules which encode the anthrax spore-associated protein, the PA protein, or immunogenic fragments and functional variants thereof. For example, the proteins or fragments thereof may be produced using cell-free translation systems and RNA molecules derived from DNA constructs that encode the such proteins or fragments. Alternatively, the proteins or fragments may be made by transfecting host cells with expression vectors that comprise a DNA sequence that encodes one of the proteins or fragments and then inducing expression of the protein or fragment thereof in the host cells. For recombinant production, recombinant constructs comprising one or more of the sequences which encode the desired protein or fragment are introduced into host cells by conventional methods such as calcium phosphate transfection, DEAE-dextran mediated transfection, transfection, microinjection, cationic lipid-mediated transfection, electroporation, transduction, scrape lading, ballistic introduction or infection.

The desired protein or fragment is then expressed in suitable host cells, such as for example, mammalian cells, yeast, bacteria, or other cells under the control of appropriate promoters using conventional techniques, as mentioned in the preceding sections. Following transformation of the suitable host strain and growth of the host strain to an appropriate cell density, the cells can be harvested by centrifugation, disrupted by physical or chemical means, and the resulting crude extract retained for further purification of the desired protein or fragment. In an alternative embodiment, the desired proteins or fragments thereof can be engineered with a secretory pathway signal such that the protein or desired fragments are secreted into the culture medium and obtained directly therefrom. Such secretion systems will be known in the art and will depend on the host cell in which the expression vector is being propagated in.

Conventional procedures for isolating recombinant proteins from transformed host cells are contemplated by the present invention. Such methods include, for example, isolation of the protein or fragments of interest by initial extraction from cell pellets or from cell culture medium, followed by salting-out, and one or more chromatography steps, including aqueous ion exchange chromatography, size exclusion chromatography steps, high performance liquid chromatography (HPLC), and affinity chromatography may be used to isolate the recombinant protein or fragment. Guidance in the procedures for protein purification can be found in the technical literature, including, for example, Methods in Enzymology, Volume 182, Guide to Protein Purification, Eds. J. Abelson, M. Simon, Academic Press, 1^{st} Edition, 1990, which is already incorporated by reference.

ii) Preparing the Immunogenic Compositions

To prepare the immunogenic compositions in accordance with one of the embodiments of the invention, it is possible to use known methods of purification, synthesis, or genetic engineering. Protein fragments, naked DNA/RNA, recombinant DNA/RNA, or messenger RNA may be incorporated into pharmaceutical compositions appropriate for the anticipated method of administration, such as excipients.

Various genetically engineered virus hosts, i.e. recombinant viruses, can be used to prepare anthrax spore-associated protein (and, optionally, PA) immunogenic compositions. Examples of recombinant virus hosts include, without limitation, vaccinia virus, recombinant canarypox, and defective adenovirus. Other suitable viral vectors include retroviruses that are packaged in cells with amphotropic host range and attenuated or defective DNA virus, such as herpes simplex virus, papillomavirus, Epstein Barr virus, and adeno-associated virus.

In one embodiment, adjuvants may be used to enhance the effectiveness of the immunogenic compositions of the invention. The term “adjuvant” as used herein refers to a compound or mixture which enhances the immune response to an antigen. Desirable characteristics of ideal adjuvants include, without limitation, lack of toxicity, ability to stimulate a long-lasting immune response, simplicity of manufacture and stability in long-term storage, synergy with other adjuvants, capability of selectively interacting with populations of antigen presenting cells (APC), ability to specifically elicit appropriate TH_{H1} or T_{H2} cell-specific immune responses, and ability to selectively increase appropriate antibody isotype levels (for example IgA) against antigens.

Exemplary adjuvants include, without limitation: (1) aluminum salts (alum), such as aluminum hydroxide, aluminum phosphate, aluminum sulfate, etc.; (2) oil-in-water emulsion formulations (WO 90/14837; WO 99/30739); (3) saponin adjuvants, such as Stimulon™ (Cambridge Bioscience, Worcester, Mass.) or particles generated therefrom such as ISCOMs (immunostimulating complexes); (4) Complete Freunds Adjuvant (CFA) and Incomplete Freunds Adjuvant (IFA); (5) cytokines, such as interleukins (IL-1, IL-2, IL-3, IL-4, IL-5, IL-6, IL-7, IL-8, IL-9, IL-10, IL-11, IL-12, IL-13, IL-16, IL-17, IL-19, IL-20, and the like), macrophage colony stimulating factor (M-CSF), tumor necrosis factor (TNF), VEGF, CD27, CD30, CD40, Fas Ligand, Placenta Growth Factor, etc.; (6) detoxified mutants of a bacterial ADP-ribosylating toxin such as a cholera toxin (CT), a pertussis toxin (PT), or an E. coli heat-labile toxin (LT), adjuvants derived from the CpG family of molecules; (7) R-848 (U.S. Pat. No. 5,352,784; WO99/29693); and (8) other substances that act as immunostimulating agents to enhance the effectiveness of the composition.

The determination of the amount of the respective components included in certain embodiments of the immunogenic compositions of the invention, such as antigen, lipoprotein, and adjuvant, as well as the preparation of those compositions, can be in accordance with standard techniques well known to those skilled in the pharmaceutical or veterinary arts. In particular, the afore-mentioned amounts and the dosages administered are determined taking into consideration such factors as the particular antigen, the lipoprotein, the adjuvant, the age, sex, weight, species and condition of the particular patient, and the route of administration.

The immunogenic compositions of the invention may be formulated by dispersing anthrax spore-associated protein (and any immunogenic fragments thereof or functional variants thereof) and, optionally, rPA or PA in the desired amount in any pharmaceutical carrier suitable for use in vaccines. Typical doses of anthrax vaccine are 0.5 mL in volume, but any volume suitable to deliver the desired amount of anthrax spore-associated protein (or any immunogenic fragments or functional variants thereof) and PA, if applicable, can be used. Any pharmaceutical excipient suitable for administration to mammals which does not interfere with the immunogenicity of the anthrax spore-associated protein (and PA, if applicable) may be employed. Example excipients include, without limitation, sterile water, physiological saline, glucose or the like.

The immunogenic compositions can also be lyophilized. The compositions can contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Compositions of the invention may be provided as liquid preparations, e.g., isotonic aqueous solutions, suspensions, emulsions or viscous compositions, which may be buffered to a selected pH. If digestive tract absorption is preferred, compositions of the invention can be in the “solid” form of pills, tablets, capsules, caplets and the like, including “solid” preparations which are time-released or which have a liquid filling, e.g., gelatin covered liquid, whereby the gelatin is dissolved in the stomach for delivery to the gut.

If nasal or respiratory (mucosal) administration is desired, compositions may be prepared as inhalables, sprays, and the like and dispensed by a squeeze spray dispenser, pump dispenser, or aerosol dispenser. Aerosols are usually under pressure by means of a hydrocarbon. Pump dispensers can preferably dispense a metered dose or, a dose having a particular particle size.

Compositions within the scope of this invention can contain a humectant to inhibit drying of the mucous membrane and to prevent irritation. Any of a variety of pharmaceutically acceptable humectants can be employed including, for example sorbitol, propylene glycol or glycerol. As with the thickeners, the concentration will vary with the selected agent, although the presence or absence of these agents, or their concentration, is not an essential feature of this invention.

Enhanced absorption across the mucosal and especially nasal membrane can be accomplished employing a pharmaceutically acceptable surfactant. Typically useful surfactants for compositions include polyoxyethylene derivatives of fatty acid partial esters of sorbitol anhydrides such as Tween 80, Polyoxynol 40 Stearate, Polyoxyethylene 50 Stearate and Octoxynol.

A pharmaceutically acceptable preservative can be employed to increase the shelf-life of the compositions. Benzyl alcohol may be suitable, although a variety of preservatives including, for example, Parabens, thimerosal, chlorobutanol, or benzalkonium chloride may also be employed.

Compositions of the invention can contain pharmaceutically acceptable flavors and/or colors for rendering them more appealing, especially if they are administered orally. The viscous compositions may be in the form of gels, lotions, ointments, creams and the like and will typically contain a sufficient amount of a thickening agent so that the viscosity is from about 2500 to 6500 cps, although more viscous compositions, even up to 10,000 cps may be employed. Viscous compositions can be formulated within the appropriate viscosity range to provide longer contact periods with mucosa, such as the lining of the stomach or nasal mucosa.

The choice of suitable carriers and other additives will depend on the exact route of administration and the nature of the particular dosage form, e.g., liquid dosage form [e.g., whether the composition is to be formulated into a solution, a suspension, gel or another liquid form, or solid dosage form [e.g., whether the composition is to be formulated into a pill, tablet, capsule, caplet, time release form or liquid-filled form].

Solutions, suspensions and gels, normally contain a major amount of water (preferably purified water) in addition to the antigen and other optional components. Minor amounts of other ingredients such as pH adjusters (e.g., a base such as NaOH), emulsifiers or dispersing agents, buffering agents, preservatives, wetting agents, jelling agents, (e.g., methylcellulose), colors and/or flavors may also be present. The compositions can be isotonic, i.e., it can have the same osmotic pressure as blood and lacrimal fluid.

The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions may be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount which will achieve the selected viscosity. Viscous compositions are normally prepared from solutions by the addition of such thickening agents.

A pharmaceutically acceptable preservative can be employed to increase the shelf-life of the compositions. Benzyl alcohol may be suitable, although a variety of preservatives including, for example, parabens, thimerosal, chlorobutanol, or benzalkonium chloride may also be employed. A suitable concentration of the preservative will be from 0.02% to 2% based on the total weight although there may be appreciable variation depending upon the agent selected.

Those skilled in the art will recognize that the components of the compositions can be selected to be chemically inert with respect to the antigen and other optional components. This will present no problem to those skilled in chemical and pharmaceutical principles. The skilled person in view of problems encountered in the formulation of the medicaments of the invention can readily reference standard technical texts or carry out experimentation which is not undue to determine the best and most appropriate manner to formulate the medicaments of the invention.

The immunologically effective compositions of this invention are prepared by mixing the ingredients following generally accepted procedures. For example the selected components may be simply mixed in a blender, or other standard device to produce a concentrated mixture which may then be adjusted to the final concentration and viscosity by the addition of water or thickening agent and possibly a buffer to control pH or an additional solute to control tonicity as in manners exemplified but not limited to the above description.

### Methods of Inducing Immunological Responses

In additional embodiments, the invention is directed to methods of using the nucleic acid-based or protein-based immunogenic compositions described above to elicit a protective immune response against lethal infection with B. anthracis or its toxins in an animal subject. The method comprises administering one of the above-described immunogenic compositions to the subject in a therapeutically effective amount. As used herein, the term “therapeutically effective amount” can mean that the amount administered can have a protective effect against pathologic consequences of infection, i.e. a therapeutic benefit. The compositions can be administered at a dosage sufficient to elicit, prime, or boost an immune response which prophylactically protects against a lethal B. anthracis infection in the animal. The animal subject may be any mammal, including a human subject. The immune response prophylactically prevents a lethal B. anthracis infection in the animal. The active immunity elicited by immunization with the above-described immunogenic compositions can prime or boost a cellular or humoral immune response.

### Administration of Immunogenic Compositions

Immunogenic compositions according to the invention may be administered to a subject in which it is desired to elicit an immune response against B. anthracis. In addition to humans, the compositions of the present invention may advantageously be administered, for example, to horses, cattle, oxen, goats, sheep, dogs, cats, antelope, buffalo, rabbits, pigs, and the like.

In one embodiment, the method of the invention comprises directly administering a nucleic acid, particularly a DNA, which encodes at least one anthrax spore-associated protein, an immunogenic fragment thereof, a functional variant thereof and optionally, PA or immunogenic and/or functional variant fragments thereof, into the subject. In another embodiment, the protein or peptide-based immunogenic compositions of the invention are administered to the animal subject.

Administration may be made in a variety of routes including, without limitation, orally, transbucally, transmucosally, sublingually, nasally, rectally, vaginally, intranasally, intraocularly, intramuscularly, intralymphatically, intravenously, subcutaneously, transdermally, intradermally, intra tumor, topically, transpulmonarily, by inhalation, by injection, or by implantation, etc. In one embodiment, the nucleic acid-based composition of the invention is introduced into muscle tissue; in other embodiments, the nucleic acid-based composition is incorporated into tissues of skin, brain, lung, liver, spleen or blood. The preparation may be placed within cavities of the body. In still other embodiments, the nucleic acid based-composition is impressed into the skin or administered by inhalation.

Means of administration further include, without limitation, gold particles coated with DNA and projected so as to penetrate into the cells of the skin of the subject to be vaccinated (Tang et al. Nature 1992.356.152-154) and the liquid jet injectors which make it possible to transfect both skin cells and cells of the underlying tissues (Furth et al. Analytical Bioch. 1992. 205.365-368).

Those skilled in the art will recognize that for injection, formulation in aqueous solutions, such as Ringer's solution or a saline buffer may be appropriate. Liposomes, emulsions, and solvents are other examples of delivery vehicles. Oral administration would require carriers suitable for capsules, tablets, liquids, pills, etc, such as sucrose, cellulose, etc.

Dosage treatment may be a single dose schedule or a multiple dose schedule. A multiple dose schedule can be one in which a primary course of vaccination may be with 1 dose, followed by another dose given at a subsequent time interval, chosen to maintain and/or reinforce the immune response. The 1 or 2 injections may be carried out over an extended period of time. Thus, in one embodiment of the immunogenic compositions of the invention, a desired anti-anthrax spore-associated protein (and, optionally, anti-PA) antibody titer is obtained in a subject with fewer doses of the immunogenic composition than the regimen employed with AVA: six doses administered over 18 months. In another embodiment, the method of the invention involves administration of 1 or 2 doses to obtain a desired anti-anthrax spore-associated protein (and, optionally, anti-PA) antibody titer in an immunized mammalian subject such as a human. In yet another embodiment, protective immunity to B. anthracis is imparted to the immunized subject.

Anti-anthrax spore-associated protein or anti-PA titer, measured as the reciprocal of the dilution of serum at which no anthrax spore-associated protein-reactive or PA-reactive antibody, respectively, is detected, is a common measure of the effectiveness of anthrax vaccines. (Pittman et al., Vaccine, 19:213-216 (2000)). The interval between repeated administrations of the immunogenic composition may vary, and judicious spacing of the doses can increase the immune response, as measured by anti-anthrax spore-associated protein or anti-PA titer. Any spacing of doses may be employed that achieves the desired immune response.

The immunogenic compositions of the invention may be administered in a dosage sufficient to prevent a lethal B. anthracis infection in a subject through a series of immunization challenge studies using a suitable animal host system, e.g. rhesus macaques, which are thought to be an acceptable standard for human use considerations. The dosage regimen will also, at least in part, be determined by the need of the subject and be dependent on the judgment of the clinician. The dosage to be administered depends on the size of the subject being treated as well as the frequency of administration and route of administration. Ultimately, the dosage will be determined using clinical trials. Initially, the clinician will administer doses that have been derived from animal studies. If prevention of disease is desired, the vaccines can generally be administered prior to primary infection with the pathogen of interest. If prevention of disease post-infection (but before germination of spores) or prevention of progression of disease, e.g., the reduction of symptoms or recurrences after infection and germination of spores, is desired, the vaccines can generally be administered within about one to about sixty days after primary infection, or after primary infection in concert with other anti-anthrax treatment, respectively.

For any composition to be administered to an animal or human, including the components thereof, and for any particular method of administration, it is preferred to determine therefor: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable immunological response, such as by titrations of sera and analysis thereof for antibodies or antigens, e.g., by ELISA. Such determinations do not require undue experimentation from the knowledge of the skilled artisan, this disclosure and the documents cited herein. As discussed above, the time frame for sequential administrations can be ascertained without undue experimentation.

### Antibodies of the Invention

The present invention also contemplates antibodies against the antigens of the invention, for example, the anthrax spore-associated proteins of the invention, and any immunogenic fragments thereof or functional variants thereof, and any suitable methods for preparing the antibodies that are available to the skilled artisan. The antibodies can be used in diagnostic methods for detecting infections of B. anthracis or the presence of B. anthracis toxins and for treating infections of B. anthracis.

Antibodies that bind the anthrax spore-associated proteins, and PA, and any immunogenic and/or functional variants thereof can be prepared by a variety of methods that are known in the art and outlined in the technical literature, for example, in Current Protocols in Molecular Biology, Ausubel, F. M. et al., (eds.) Greene Publishing Associates, (1989), Chapter 2. As one example of such methods, a preparation of an anthrax spore-associated protein of the invention or immunogenic and/or functional variant thereof is prepared and purified to render it substantially free of natural contaminants. Such a preparation is then introduced into an animal in order to produce polyclonal antisera of greater specific activity.

Monoclonal antibodies specific for the proteins of the invention, or immunogenic and/or functional variants thereof can be prepared using hybridoma technology (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 563-681 (1981)). In general, an animal (e.g. a mouse) is immunized with a protein of the invention. The splenocytes of such mice are extracted and fused with a suitable myeloma cell line. Any suitable myeloma cell line may be employed in accordance with the present invention; however, for example, the parent myeloma cell line (SP2O), available from the ATCC. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding a the proteins of the invention, e.g. the anthrax spore-associated proteins or immunogenic and/or functional variants thereof of the invention.

Alternatively, additional antibodies capable of binding to proteins of the invention can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the protein of the invention-specific antibody can be blocked by the protein of the invention. Such antibodies comprise anti-idiotypic antibodies to the protein of the invention-specific antibody and are used to immunize an animal to induce formation of further protein of the invention-specific antibodies.

For in vivo use of antibodies in humans, an antibody can be “humanized”. Such antibodies can be produced using genetic constructs derived from hybridoma cells producing the monoclonal antibodies described above. Methods for producing chimeric and humanized antibodies are known in the art and are discussed herein. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., International Publication No. WO 8702671; Boulianne et al., Nature 312:643 (1984); Neuberger et al., Nature 314:268 (1985)), each of which are incorporated by reference in their entireties.

The present invention further contemplates diagnostic methods which use the antibodies of the invention, e.g. those directed against the anthrax spore-associated proteins or immunogenic fragments and/or functional variants thereof, to diagnose an infection of B. anthracis or the presence of a B. anthracis toxin. In one aspect, the present invention contemplates an immunoassay that tests a subjects blood or tissues using the antibodies of the invention to detect or determine whether the blood or tissue comprises B. anthracis spores, whole bacteria, or toxins thereof. The antibodies can be provided in the form of a diagnostic kit, which can include other necessary or desirable components, such as sterile vessels for reacting the blood/tissue with the antibodies, antibodies, syringes or other advantageous implements or instruments, and any necessary or desirable reagents.

The instant invention further contemplates pharmaceutical compositions comprising the antibodies of the invention in a therapeutically effective dose or quantity and any desirable or advantageous excipients. Pharmaceutical compositions have been described above. The pharmaceutical compositions comprising the antibodies of the invention can be administered to a subject in need thereof, e.g. a patient or animal infected with B. anthracis, by any means known to the skilled artisan and as described herein.

### Kits of the Invention

In one embodiment, the invention provides kits containing the immunogenic compositions of the invention and instructions for admixture and/or administration. The kits can comprise the polypeptide-based compositions (e.g. a therapeutically effective dose of an anthrax spore-associated protein of the present invention, or an immunogenic fragment or functional variant thereof), or nucleic-acid compositions (e.g. a nucleotide vector encoding an anthrax spore-associated protein of the invention, or an immunogenic fragment or functional variant thereof), or a combination of both. In the case of the combination, the kit can comprise separate vessels of the polypeptide-based compositions and the nucleic-acid based compositions or alternatively, such compositions can be combined together in a suitable admixture. In one embodiment, the invention provides a kit comprising an immunogenic composition comprising at least one anthrax spore-associated protein or an immunogenic composition comprising at least one expression vector, wherein the expression vector contains a nucleic acid molecule encoding an anthrax spore-associated protein or fragment thereof and instructions for administering the immunogenic composition to induce an immunological response in a subject.

The kits contemplated by the invention can also contain any implement for the successful and complete delivery of the compositions of the invention, such as, but not limited to, a syringe, sterile mixing vessel, measuring device, and instructions, etc. The kits of the invention are also not limited to the provision of a single dose or delivery of the compositions of the present invention, but can contain any suitable quantity of doses, such as, a suitable quantity of compositions to last 1 week, 1 month, or 1 year or more.

Any of the compositions of the kits of the invention can also include other suitable polypeptides or polypeptide-encoding nucleotide vectors of the invention, such as B. anthracis PA or an immunogenic fragment or functional variant thereof.

While the description above refers to particular embodiments of the present invention, it will be understood that many modifications may be made without departing from the spirit thereof. The accompanying claims are intended to cover such modifications as would fall within the true scope and spirit of the present invention.

The present invention is additionally described by way of the following illustrative, non-limiting Examples that provide a better understanding of the present invention and of its many advantages.

### EXAMPLES

### Example 1

### Generation of a Limited Expression Library of Anthrax Spore-Associated Proteins

An inducible, B. anthracis genomic DNA expression library was first constructed using genomic DNA isolated from the non-pathogenic B. anthracis Sterne strain in the pET30 (abc) series of expression vectors (which permit cloning of inserts in each of three reading frames under the control of the T7 phage promoter), and the expression host E. coli BL21 (DE3) (Novagen, Madison, Wis.). A limited expression library of putative anthrax spore-surface (spore-associated) proteins was then generated by screening the above genomic expression library with affinity-purified, polyclonal antibodies generated against a mixture of gamma-irradiated, purified, intact spores produced by B. anthracis Vollum, Ames and Sterne strains, in goats (Chemicon, Temecula, Calif.). A total of 292 reactive clones were identified (unpublished data), and comprised the limited, expression library of anthrax spore-associated proteins that was probed with sera from AVA-vaccinated humans (see below) in this study.

### Pre-Immune and Immune Human Sera.

Pre-immune and immune serum samples were collected from two human adult volunteers immunized with AVA at the Division of Infectious Diseases, Massachusetts General Hospital, Boston, Mass. The institutional review board (IRB) of the Massachusetts General Hospital approved the collection and use of these serum samples. Specifically, serum samples (10 ml) were collected prior to the first administration (pre-immune) and two weeks following the fourth administration (dose administered at six months) of AVA (immune sera). Sera from this time point were utilized as a probe for the screen, since results of experiments in non-human primates indicate that protective immunity against inhalational anthrax is engendered following two administrations of AVA (Friedlander, A. M., et al., 1999. JAMA 282:2104-2106). Serum samples were dispensed in small volumes and stored at −70° C. until used.

### Preparation of Pre-Immune and Immune Sera for Screening the Limited Expression Library of Anthrax Spore-Associated Proteins.

Prior to use as probes, sera were pooled to compensate for variations in immune responses of individuals and to identify a wider array of reactive spore-associated proteins, and were used either directly (crude sera) or following affinity purification (affinity-purified sera). Sera were affinity-purified using magnetic beads linked to either Protein A or Protein G (Dynabeads Protein A or Dynabeads Protein G, respectively), as per the instructions of the manufacturer (Dynal Biotech, Lake Success, N.Y.), with modifications. Protein A reportedly binds all human immunoglobulin (Ig) isotypes and IgG subclasses except IgG3, whereas Protein G binds all IgG subclasses but not other Ig isotypes (Ed Harlow and David Lane. 1988. Cold Spring Harbor Laboratory Press, Cold Spring Harbor, N.Y.). Initially, pooled sera were affinity-purified using both Dynabeads Protein A and Dynabeads Protein G; however, pilot colony immunoblotting experiments revealed that the pooled sera affinity purified using Dynabeads Protein A consistently yielded better results (data not shown), and this affinity-purified sera was therefore used as a probe in subsequent colony immunoblotting experiments.

For capture of antibodies by Dynabeads Protein A, 10 μl of pooled pre-immune or immune sera was added to 100 μl of beads, prepared as instructed by the manufacturer, and incubated at room temperature with slow tilt rotation for 30 min. The beads were then pulled down using a magnet, the supernatant decanted, and beads washed as instructed by the manufacturer to remove loosely bound components. Specifically bound Igs were eluted with 0.1 M citrate (pH 3.0) directly into 1 M Tris (pH 9.0). Crude and affinity-purified sera were stored at 4° C. following the addition of 0.02% sodium azide until further use. Long-term storage was in 50% glycerol at −70° C.

### Assessment of the Quality of Pooled Sera.

Because human-use acellular PA-based vaccines were found to induce weak and inconsistent immune responses (Hambleton, P., et al., 1984. Vaccine 2:125-13229; Lincoln, R. and D C Fish. 1970. Anthrax toxin, p. 361-414. In T. C. Monte, et al., Academic Press, Inc., New York), and due to the lack of standardization of the vaccine manufacturing process (Leppla, S. et al., 2002. J. Clin. Invest. 110:141-144), the quality of pooled, immune sera was examined prior to use as a probe for screening the previously generated limited expression library of anthrax spore-associated proteins. The quality of crude and affinity-purified sera was assessed by reacting pooled, pre-immune and immune sera with a recombinant (test) clone, E. coli BL21 (DE3)(pSMR-PA), expressing full-length PA utilizing a colony immunoblot assay. Reactivity against this particular protein was examined, since PA reportedly is the principal immunogen and a major component of AVA (Leppla, S. H., et al, J. Clin. Invest. 110:141-144), and anti-PA antibodies are a gauge of the host response to immunization (Joellenbeck, L. M., et al., 2002. National Academy Press, Washington, D.C.).

For immunoscreening, the test clone and E. coli BL21 (DE3) (pET30a) (negative control) were tooth-picked on duplicate Luria-Bertani (LB) plates supplemented with 50 μg/ml of kanamycin (LB-Kan) and incubated overnight at room temperature. Colonies were lifted from one of the plates (the other plate constituted the “Master” plate) using a nitrocellulose filter and placed colony side up on a fresh LB-Kan plate containing 1 mM isopropyl-β-D-thiogalactoside (IPTG). Following an overnight incubation at 30° C. to induce expression of genes contained within cloned inserts, colonies on plates were partially lysed by exposing them to chloroform vapors for 15 min in a candle jar. The filters were then removed from the plates, air dried, and blocked using 5% non-fat milk in phosphate buffered saline (pH 7.4) (PBS) for 1 h at room temperature. After rinsing with PBS containing 0.05% Tween 20 (PBS-T), filters were probed with a 1:5,000 dilution of either pooled, crude pre-immune or immune sera, or with a 1:500 dilution of either pooled, affinity-purified pre-immune or immune sera. Following an overnight incubation at 4° C. on a rocking platform, filters were washed 3× with PBS-T, and incubated with a 1:20,000 dilution of peroxidase-labeled goat IgG raised against the human gamma globulin fraction (ICN/Cappel, Aurora, Ohio). Filters were developed using an ECL chemiluminescence kit (Amersham Biosciences), and positive clones were identified by their positions on the “Master” plate.

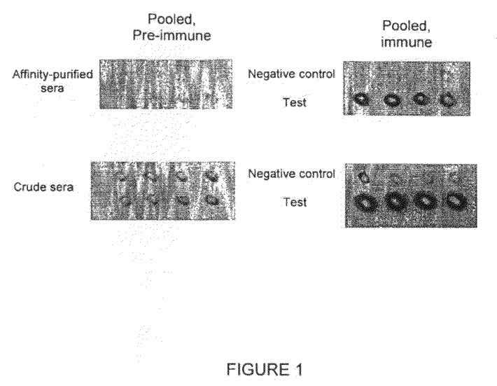

As shown in FIG. 1, the recombinant clone expressing full-length PA was strongly reactive with pooled, crude and affinity-purified immune sera but not with pooled, crude or affinity purified pre-immune sera, indicating a robust immune response to AVA. That this reactivity was specific was also evidenced by the result that neither the crude nor affinity-purified immune serum pool reacted with the negative control, which consisted of E. coli BL21 (DE3) host strain carrying the native, non-recombinant expression plasmid vector, pET30a (the same host-vector combination used in the construction of the B. anthracis expression library). These results indicated that the pooled pre-immune and immune sera were suitable for probing the limited expression library of anthrax spore-associated proteins.

### Screening of the Limited, Expression Library of Anthrax Spore-Associated Proteins by Colony-Immunoblotting.

It was previously reported that unidentified antigens might significantly contribute to the protective immunity of PA-based vaccines (Brossier, F., et al., 2002. Infect. Immun. 70:661-664; Cohen, S. I., et al., 2000. Infect. Immun. 68:4549-4558; Little, S. F. and G. B. Knudson. 1986. Infect. Immun. 52:509-512; Pezard, C., et al., 1995. Infect. Immun. 63:1369-1372; Stepanov, A., et al., 1996. J. Biotechnol 44:155-160; Welkos, S., et al., 2001. Microbiology 147:1677-1685). It was also documented that immunization with AVA induces protective immunity against both cutaneous (Joellenbeck, L. M., et al., 2002. National Academy Press, Washington, D.C.; Leppla, S. H., et al., 2002. J. Clin. Invest. 110:141-144) and inhalational anthrax (Friedlander, A. M., et al., 1999. JAMA 282:2104-2106; Joellenbeck, L. M., et al., 2002. National Academy Press, Washington, D.C.; Leppla, S. H., et al., 2002. J. Clin. Invest. 110:141-144), albeit following multiple administrations. It was, thus, investigated whether a subset of 292 library clones expressing anthrax spore-surface proteins, identified might be part of the B. anthracis immunome in patients immunized with AVA. The reactivity of recombinant clones was examined by expressing these proteins with pooled, crude and affinity-purified pre-immune and immune sera from two human adult volunteers administered four doses of AVA.

Prior to screening, each of the 292 clones expressing spore-associated proteins was tooth-picked on duplicate LB-Kan plates in a grid pattern alternating with the negative control, and incubated at 37° C. for 6 h. Colonies were lifted, and induction of gene expression from cloned inserts was performed as described above. The filters were processed as detailed earlier and probed with a 1:10,000 dilution of pooled, crude pre-immune or immune sera at 37° C. for 1 h. Filters were then washed 3× with PBS-T, and incubated with a 1:20,000 dilution of peroxidase-labeled goat IgG raised against human gamma globulin fraction (ICN/Cappel) for 1 h at 37° C. Filters were washed and developed as before, and reactive clones were identified by their positions on the “Master” plate. Positive clones were purified and reactivity confirmed via an additional round of colony immunoblotting using pooled, affinity-purified pre-immune and immune sera at a dilution of 1:500 using the same procedure described in the previous section for screening the test clone and the negative control. This immunological screen resulted in the identification of 69 expression library clones expressing proteins that were targets of AVA-induced immunity.

Identification of Anthrax Spore-Associated Proteins Reactive with Immune Sera.

To identify proteins expressed from each clone, lysates of each positive clone were prepared as described earlier (Kudva, I. T., et al., 2002. J. Bacteriol. 184:1873-18791) and used as a template in PCR. Amplification reactions were performed using vector-specific primers obtained from the DNA Synthesis Core Facility, Department of Molecular Biology, Massachusetts General Hospital as described earlier (Kudva, I. T., et al., 2002. J. Bacteriol. 184:1873-1879). Amplicons were purified using the QIAQuick PCR Purification Kit (Qiagen, Valencia, Calif.) and subjected to DNA sequencing at the DNA Sequencing Core Facility, Department of Molecular Biology, Massachusetts General Hospital, using an ABI Prism DiTerminator cycle sequencing with AmpliTaq DNA polymerase FS with an ABI 377 DNA sequencer (Perkin-Elmer Applied Biosystems Division, Foster City, Calif.).

Genes on cloned inserts within reactive clones were identified via BLAST by comparing nucleotide sequences against those contained in the non-redundant database at the National Center for Biotechnology information (NCBI), and against sequences of B. anthracis Ames strain in the database at The Institute for Genomic Research (TIGR). Protein identities and functions (Table 1) were determined from the TIGR database and Swiss-Prot/trEMBL databases, or the NCBI's Conserved Domain Database (CDD; Marchler-Bauer, A. and S. H. Bryant. 2004. Nucleic Acids Res. 32:W327-W331).

TABLE 1,
Anthrax spore-associated proteins reactive with
sera of human adults immunized with AVA

| | | B. anthracis | B. anthracis | |
|---|---|---|---|---|
| Functional | Clone | Sterne | Ames | |
| Category¹ | Number | Locus ID | Locus ID | Gene/Protein/Function⁴ |
| Protein Synthesis, | 14 | BAS0087 | BA0086 | gltx/GltX; glutamyl- |
| Modification, Repair: | | | | tRNA synthetase/ |
| | | | | tRNA aminoacylation |
| | 109 | BAS3884 | BA4187 | def-1/Def-1; polypeptide |
| | | | | deformylase/ |
| | | | | protein modification and |
| | | | | repair |
| | 368² | BAS4955 | BA5332 | smpB/SmpB; ssrA-binding |
| | | | | protein/ |
| | | | | protein synthesis: binds |
| | | | | specifically |
| | | | | to the ssrA RNA (tmRNA) |
| | | | | and required for stable |
| | | | | association of ssrA with |
| | | | | ribosomes |
| | | BAS4956 | BA5334 | vacB/VacB; ribonuclease |
| | | | | R/transcription: RNA |
| | | | | processing |
| | 1188 | BAS5178 | BA5572 | prfA/PrfA; peptide chain |
| | | | | release factor I/ |
| | | | | translation: peptide chain |
| | | | | release factor I directs the |
| | | | | termination of translation |
| | | | | in response to the peptide |
| | | | | chain termination codons |
| | | | | UAG and UAA |
| | 1262² | BAS0016 | BA0013 | None/None/sigma70_4; |
| | | | | region 4 of sigma-factor |
| | | | | 70)/binding to the −35 |
| | | | | promoter element via a |
| | | | | helix-turn-helix motif)⁵ |
| | | BAS0015 | BA0012 | serS/SerS; seryl-tRNA |
| | | | | synthetase/tRNA |
| | | | | aminoacylation |
| Transport and | 103² | BAS0206 | BA0210 | None/None; transporter, |
| Binding: | | | | EamA family/transport |
| | | | | and binding of proteins |
| | | BAS0205 | BA0208 | None/None; |
| | | | | transcriptional regulator - |
| | | | | LysR family/DNA |
| | | | | interactions |
| | 268 | BAS1105 | BA1195 | None/None; oligopeptide |
| | | | | ABC transporter, ATP |
| | | | | binding protein/transport |
| | | | | and binding of amino |
| | | | | acids, peptides and amines |
| | 373² | BAS3376 | BA3641 | None/none; rADc, |
| | | | | ribosomal RNA adenine |
| | | | | dimethylases)/ |
| | | | | methylation of an adenine |
| | | | | of ribosomal RNA⁵ |
| | | BAS3377 | BA3642 | None/None; oligopeptide |
| | | | | ABC transporter, |
| | | | | oligopeptide binding |
| | | | | protein/transport and |
| | | | | binding of amino acids, |
| | | | | peptides and amines |
| | 824 | BAS4394 | BA4734 | None/None; oligopeptide |
| | | | | ABC transporter, ATP |
| | | | | binding protein/transport |
| | | | | and binding of amino |
| | | | | acids, peptides and amines |
| | 1077² | BAS4648 | BA5003 | None/None; ABC |
| | | | | transporter, putative ATP |
| | | | | binding protein/transport |
| | | | | and binding of unknown |
| | | | | substrates |
| | | BAS4647 | BA5002 | None/None; conserved |
| | | | | hypothetical protein |
| | | | | (putative rRNA methylase)/ |
| | | | | rRNA methylation |
| | 1104² | BAS3035 | BA3268 | None/None; conserved |
| | | | | hypothetical protein/ |
| | | | | unknown |
| | | BAS3034 | BA3267 | None/None; major |
| | | | | facilitator family protein/ |
| | | | | sugar transport (specific |
| | | | | substrate unknown) |
| | 164 | BAS2639 | BA2830 | None/None; |
| | | | | sodium/alanine symporter |
| | | | | family protein/ion/amino |
| | | | | acid transport |
| Cell envelope: | 151 | BAS1932 | BA2079 | dal-2/Dal-2; alanine |
| | | | | racemase/biosynthesis of |
| | | | | murein sacculus and |
| | | | | peptidoglycan |
| | 232 | BAS5205 | BA5604 | None/None; LPXTG- |
| | | | | motif (SEQ ID NO: 161) |
| | | | | cell wall anchor domain |
| | | | | protein containing a |
| | | | | collagen binding domain/ |
| | | | | unknown |
| | 367 | BAS5183 | BA5578 | mur2/MurA2; UDP-N- |
| | | | | acetylglucosamine 1- |
| | | | | carboxyvinyltransferase 2/ |
| | | | | biosynthesis of murein |
| | | | | sacculus and |
| | | | | peptidoglycan |
| | 380 | BAS5217 | BA5615 | None/None; membrane |
| | | | | protein PfoR component of |
| | | | | the sugar |
| | | | | phosphotransferase |
| | | | | system, sucrose/fructose- |
| | | | | specific/carbohydrate |
| | | | | transport and metabolism |
| | 545 | BAS1477 | BA1593 | None/None; putative |
| | | | | membrane protein/ |
| | | | | unknown |
| | 739 | BAS1135 | BA1228 | None/None; glucose-1- |
| | | | | phosphate |
| | | | | thymidylyltransferase, |
| | | | | putative/biosynthesis and |
| | | | | degradation of surface |
| | | | | polysaccharides and |
| | | | | lipopolysaccharides |
| | 862 | BAS5285 | BA5681 | None/None; membrane |
| | | | | protein, putative/ |
| | | | | unknown |
| | 812² | BAS0638 | BA0672 | inhA/InhA; immune |
| | | | | inhibitor A |
| | | | | metalloprotease/ |
| | | | | metallopeptidase |
| | | | | functioning in proteolysis, |
| | | | | peptidolysis |
| | | BAS0637 | BA0670 | None/None; transaldolase/ |
| | | | | functions in the pentose |
| | | | | phosphate pathway |
| | 355 | BAS1246 | BA1346 | None/None; internalin, |
| | | | | putative/pathogenesis |
| | 239 | BAS4444 | BA4789 | None/None; LPXTG- |
| | | | | motif (SEQ ID NO: 161) |
| | | | | cell wall anchor domain |
| | | | | protein containing a |
| | | | | “NEAT” domain/ |
| | | | | unknown |
| Sporulation and | 435 | BAS4338 | BA4672 | obG/ObG; Spo0B- |
| Germination: | | | | associated GTP binding |
| | | | | protein/sporulation and |
| | | | | germination |
| | 528 | BAS4236 | BA4566 | sigK/SigK; transcription |
| | | | | factor/sporulation and |
| | | | | germination |
| Metabolism: | 19 | BAS4413 | BA4754 | sdhA/SdhA; succinate |
| | (Identified | | | dehydrogenase, |
| | twice) | | | flavoprotein subunit/ |
| | | | | tricarboxylic acid (TCA) |
| | | | | cycl |
| | 195 | BAS2768 | BA2980 | None/None; carbohydrate |
| | | | | kinase, FGGY family, |
| | | | | authentic frameshift/ |
| | | | | sugar metabolism |
| | 243² | BAS0672 | BA0706 | None/None; N-acyl-L- |
| | | | | amino acid |
| | | | | amidohydrolase (peptidase |
| | | | | family M20)/metabolism |
| | | | | of amino acids and amines |
| | | BAS0673 | BA0707 | None/None; conserved |
| | | | | hypothetical protein |
| | 255 | BAS4315 | BA4650 | ruvB/RuvB; DNA |
| | | | | replication, recombination, |
| | | | | and repair |
| | 404 | BAS5149 | BA5541 | muoB/NuoB; NADH |
| | | | | dehydrogenase I, B subunit/ |
| | | | | electron transport |
| | 777 | BAS4186 | BA4508 | nfo/Nfo; endonuclease IV/ |
| | | | | DNA replication, |
| | | | | recombination, and repair |
| | 829² | BAS4875 | BA5246 | None/None; acyl-CoA |
| | | | | dehydrogenase/ |
| | | | | degradation of fatty acids |
| | | | | and phospholipids |
| | | BAS4876 | BA5248 | None/None; acetyl-CoA |
| | | | | acetyltransferase/fatty |
| | | | | acid and phospholipid |
| | | | | metabolism |
| | 1091 | BAS2724 | BA2932 | None/None; putative |
| | | | | glutathionylspermidine |
| | | | | synthase/polyamine |
| | | | | biosynthesis |
| | 1111² | BAS1283 | BA1385 | None/None; 2- |
| | | | | nitropropane dioxygenase/ |
| | | | | nitrogen metabolism |
| | | | | (oxidoreductase activity) |
| | | BAS1282 | BA1384 | None/None; |
| | | | | peptidase_U61, LD- |
| | | | | carboxypeptidase)/ |
| | | | | hydrolysis of peptide bond |
| | | | | between a di-basic amino |
| | | | | acid and the C-terminal D- |
| | | | | alanine in the tetrapeptide |
| | | | | moiety in peptidoglycan; |
| | | | | murein recycling⁵ |
| | 1264 | BAS4563 | BA4918 | acuC/AcuC; acetoin |
| | | | | utilization protein/acetoin |
| | | | | catabolism |
| | 165 | BAS4985 | BA5364 | eno/Eno; enolase/ |
| | | | | glycolysis/gluconeogenesis |
| Amino acid | 218 | BAS0331 | BA0346 | None/None; 5- |
| Biosynthesis: | (Identified | | | methylthioribose kinase, |
| | twice) | | | putative/biosynthesis of |
| | | | | aspartate family |
| | 341² | BAS4039 | BA4354 | argJ/ArgJ; glutamate N- |
| | | | | acetyltransferase/amino- |
| | | | | acid acetyltransferase/ |
| | | | | biosynthesis of glutamate |
| | | | | family |
| | | BAS4040 | BA4355 | argC/ArgC; N-acetyl- |
| | | | | gamma-glutamyl- |
| | | | | phosphate reductase/ |
| | | | | biosynthesis of glutamate |
| | | | | family |
| | 756 | BAS1676 | BA1811 | dapG-1/DapG-1; |
| | | | | aspartate kinase, |
| | | | | monofunctional class/ |
| | | | | biosynthesis of aspartate |
| | | | | family |
| Nucleoside/Nucleotide | 234 | BAS3739 | BA4027 | pyrC/PyrC; |
| Biosynthesis: | | | | dihydroorotase/ |
| | | | | pyrimidine ribonucleotide |
| | | | | biosynthesis |
| | 22 | BAS4303 | BA4638 | apt/Apt; adenine |
| | | | | phosphoribosyltransferase/ |
| | | | | salvage of nucleosides |
| | | | | and nucleotides |
| | 387² | BAS5179 | BA5573 | tdk/Tdk; thymidine |
| | | | | kinase/nucleotide and |
| | | | | nucleoside |
| | | | | interconversions |
| | | BAS5180 | BA5574 | rpmE/RpmE; ribosomal |
| | | | | protein L31/synthesis and |
| | | | | modification of ribosomal |
| | | | | proteins |
| | 1284 | BAS0286 | BA0299 | purD/PurD; |
| | (Identified | | | phosphoribosylamine-- |
| | twice) | | | glycine ligase/purine |
| | | | | ribonucleotide |
| | | | | biosynthesis |
| Biosynthesis of | 26 | BAS1986 | BA2134 | None/None; |
| cofactors, prosthetic | | | | molybdopterin |
| groups, and carriers: | | | | biosynthesis protein, |
| | | | | putative/molybdopterin |
| | | | | biosynthesis |
| | 1295 | BAS5244 | BA5463 | thiC/ThiC; thiamine |
| | | | | biosynthesis protein/ |
| | | | | thiamine biosynthesis |
| | 753 | BAS0698 | BA0732 | thiG/ThiG; thiazole |
| | | | | biosynthesis protein/ |
| | | | | thiamine biosynthesis |
| | | BAS0697 | BA0731 | thiS/ThiS; sulphur |
| | | | | transfer protein/thiamine |
| | | | | biosynthesis |
| | 1277 | BAS1423 | BA1534 | menG/MenG; 2- |
| | | | | heptaprenyl-1,4- |
| | | | | naphthoquinone |
| | | | | methyltransferase/ |
| | | | | biosynthesis of |
| | | | | menaquinone and |
| | | | | ubiquinone |
| Protein degradation: | 223 | BAS4052 | BA4368 | pepT-2/PepT-2; peptidase |
| | (Identified | | | T/degradation of proteins, |
| | twice) | | | peptides, and |
| | | | | glycopeptides |
| | 304 | BAS5208 | BA5606 | None/None; |
| | | | | aminopeptidase, putative/ |
| | | | | degradation of proteins, |
| | | | | peptides, and |
| | | | | glycopeptides |
| | 369 | BAS2981 | BA3206 | None/None; |
| | | | | amidohydrolase family |
| | | | | protein, peptidase family |
| | | | | M20/M25/M40/ |
| | | | | degradation of proteins, |
| | | | | peptides, and |
| | | | | glycopeptides⁵ |
| | 72 | BAS0167 | BA0165 | None/None; prolyl |
| | | | | oligopeptidase family |
| | | | | protein, putative |
| | | | | (dipeptidyl peptidase type |
| | | | | IV)/degradation of |
| | | | | proteins, peptides, and |
| | | | | glycopeptides |
| Regulatory proteins: | 1031 | BAS4706 | BA5067 | None/None; sensory box |
| | | | | histidine kinase/Protein |
| | | | | interactions, component of |
| | | | | two-component signal |
| | | | | transduction system |
| | 1152 | BAS4548 | BA4902 | None/None; |
| | | | | transcriptional regulator, |
| | | | | LysR family/DNA |
| | | | | interactions |
| Other functions: | 398 | BAS3747 | BA4035 | divIVA/DivIVA; cell- |
| | | | | division initiation protein |
| | | | | DivIVA/cell division |
| | 1284 | BAS0803 | BA0843 | None/None; Catalase/ |
| | (Identified | | | detoxification |
| | twice) | | | |
| Unknown function: | 80² | BAS2069 | BA2225 | None/None; |
| | | | | acetyltransferase, GNAT |
| | | | | family |
| | | BAS2071 | BA2226 | None/None; hypothetical |
| | | | | protein (histidinol- |
| | | | | phosphatase, N-terminal)/ |
| | | | | amino acid transport and |
| | | | | metabolism⁵ |
| | 746 | BAS4590 | BA4946 | None/None; conserved |
| | | | | hypothetical protein |
| | | | | (DUF84 family) |
| | 239 | BAS3944 | BA4253 | None/None; hydrolase, |
| | | | | carbon-nitrogen family |
| | 473 | BAS2375 | BA2552 | None/None; carboxyl |
| | | | | transferase domain protein, |
| | | | | biotin carboxylase activity |
| | 925² | BAS0485 | BA0514 | None/None; |
| | | | | chlorohydrolase family |
| | | | | protein (Metallo- |
| | | | | dependent hydrolases, |
| | | | | subgroup C) |
| | | BAS0484 | BA0513 | None/None; RimL family |
| | | | | of acetyltransferases/ |
| | | | | acetylation of N-terminal |
| | | | | serine of 30S ribosomal |
| | | | | subunit protein L7; acetyl |
| | | | | transferase⁵ |
| | 941a | BAS2700 | BA2899 | None/None; |
| | | | | aminotransferase, classes I |
| | | | | and II |
| | 941b | BAS2061 | BA2217 | None/None; hydrolase, |
| | | | | alpha/beta fold family, |
| | | | | aminopeptidase |
| | 1038 | BAS4188 | BA4510 | None/vrrA protein |
| | 345 | BAS3788 | BA4077 | None/None; reovirus |
| | | | | sigma C capsid protein⁵ |
| | 1272 | BAS5123³ | BA5515³ | None/None; hypothetical |
| | | | | protein |

¹Functional categories are based on The Institute of Genomic Research (TIGR) database grouping of proteins of the sequenced B. anthracis Ames strain.
²Two genes present on the same cloned insert.
³Encoded by a gene with no significant homology to database entries.
⁴Functions of identified proteins are as designated in the TIGR database or/and in Swiss-Prot.
⁵Putative functions of conserved hypothetical proteins determined using the Conserved Domain Database (CDD)

The anthrax spore immunome in vaccinated humans comprised of several proteins involved in protein synthesis, modification and repair (Table 1). Included within this group were clones expressing a glutamyl-tRNA synthetase (GltS), and a seryl-tRNA synthetase (SerS), both of which catalyze the attachment of specific amino acids to cognate tRNAs (tRNA aminoacylation). Of note, tRNA synthetases reportedly are present on the anthrax spore-surface (Liu, H., et al., 2004. J. Bacteriol. 186:164-178) although the precise function of such proteins in this location is unclear. Also identified was a clone expressing a polypeptide deformylase, Def-1. The deformylation it catalyzes of polypeptide chains is imperative for protein maturation, which in turn is essential for bacterial cell viability.

One protein expressed by a clone in this group was an unique RNA binding protein called SmpB, which binds with high affinity to a tmRNA molecule (functions both as a tRNA and a mRNA) encoded by ssrA (SsrA RNA) (Karzai, A. W., et al., 1999. EMBO J. 18:3793-3799) to form a complex that functions in ridding the bacterial cell of incompletely synthesized, nascent polypeptides. SmpB as a spore-associated protein may play a role in the virulence of B. anthracis. Because bacterial cells lacking tmRNA demonstrate increased sensitivity to inhibitors of protein synthesis (de la Cruz, J. and A. Vioque. 2001. RNA 7:1708-1716), SmpB may also have potential as a target for drug design. Another protein identified was the peptide chain release factor I (PrfA), a small protein that directs termination of translation in response to stop codons.

Transport and binding proteins included components of the ATP-binding cassette (ABC) superfamily, as well as members of the major facilitator superfamily (MFS). Specifically identified in this study were clones expressing components of several ABC-type transporters involved in the uptake and transport of oligopeptides. Such proteins function in Gram positive bacteria in sensing extracellular signaling molecules essential for the initiation of competence and sporulation in B. subtilis (Perego, M., et al., 1991. Mol. Microbiol. 5:173-185, Rudner D Z, et al., 1991. J. Bacteriol. 173:1388-1398), and promoting growth of Listeria monocytogenes at low temperatures and within macrophages (Borezee, E., et al., 2000. Infect. Immun. 68:7069-7077). Also identified was a clone expressing a sugar transporter (specific substrate unknown) that belonged to the MFS and another clone expressing an efflux transporter of the EamA-type, which, in E. coli, serves to regulate the level of metabolites by effluxing excess metabolites of the cysteine pathway out of the cell, which would otherwise disrupt metabolism (Franke, I., A. et al., 2003. J. Bacteriol. 185:1161-1166).

Two conserved hypothetical proteins were encoded by genes on inserts within clone #373 and clone #1077, both of which were predicted by the CDD to have S-adenosylmethioinine (SAM)-dependent methyl transferase activity. Rounding off this group was a clone expressing an integral membrane protein of the sodium:alanine symporter family (SAF). Although L-alanine is a documented spore germinant (Ireland, J. A. W. and P. C. Hanna. 2002. J. Bacteriol. 184:296-1303; Titball, R. W. and R. J. Manchee. 1987. J. Appl. Bacteriol. 62:269-273), it is currently unclear whether this symporter plays a role in spore germination following host infection.

Cell envelope proteins included orthologs of proteins implicated in the pathogenesis of other Gram positive organisms. The screen identified clones expressing proteins possessing the C-terminal LPXTG motif (SEQ ID NO: 161), a sorting signal that anchors proteins to the cell-envelope through the action of a membrane-bound cysteine protease called sortase (Lee, V. T. and O, Schneewind. 2001. Genes & Dev. 15:1725-1752). Cell-wall anchored proteins reportedly contribute to virulence of Gram positive pathogens (Xu, Y., et al., 2004. J. Biol. Chem. 279:51760-51768) and may also play a role in B. anthracis virulence. The screen identified a clone expressing a putative internalin (InlA) protein (two paralogs, namely, BA1346 and BA0552, are present in the sequenced B. anthracis Ames strain). Such spore-associated proteins may facilitate heretofore unidentified interactions between the anthrax spore and its environment, and, therefore, are likely candidates for both vaccine and drug development.

Two other clones expressing LPXTG-domain (SEQ ID NO: 161) containing proteins were also identified. The open reading frame of one of these (BAS5205/BA5604) was disrupted, but nevertheless included a collagen-binding domain. Since collagen is a primary component of the mammalian extracellular matrix, such proteins could facilitate attachment and interaction of vegetative bacilli or spores to host connective tissues. The other LPXTG-containing (SEQ ID NO: 161) protein contained a domain that is found in the vicinity of Fe³⁺ siderophore transporters called the “NEAT” (near transporter repeat) domain (Andrade, M. A., et al, 2002. Gen. Biol. 3:RESEARCH0047). Because of the association of NEAT domains with transporters functioning in iron acquisition and transport, a requisite for survival within the mammalian host, such proteins may play a major role in disease pathogenesis. Two clones identified expressing cell envelope proteins were an UDP-N-acetylglucosamine 1-carboxyvinyltransferase 2 (MurA2) essential for the conversion of UDP-N-acetyl glucosamine into precursors for murein for peptidoglycan cell wall biosynthesis (Bernhardt, T. G., et al., 2001. Science 292:2326-2329) and a putative glucose-1-phosphate thymidylyltransferase involved in the synthesis of deoxy-thymidine diphosphate (dTDP)-L-rhamnose, a precursor of L-rhamnose, which is a component of surface structures of both Gram positive and Gram negative bacteria such as cell wall and capsular antigens known to modulate virulence and mediate attachment to host tissues (Blankenfeldt, W. M., et al., 2000. EMBO J. 19:6652-6663). Also identified was a clone expressing a predicted membrane protein, PfoR, related to membrane components of the fructose and sucrose-specific phosphotransferase systems. BLAST analysis revealed the presence of orthologs in both B. cereus and B. thuringiensis that were annotated as possible regulatory proteins. As a spore-associated protein, PfoR may have a role in spore-germination.

The screen identified a clone expressing alanine racemase, a component of the surface of anthrax spores, as well as spores produced by other members of the B. cereus family (Steichen, C. P., et al., 2003. J. Bacteriol. 185:1903-1910). This enzyme may influence the rate of spore germination (Kanda-Nambu, K. et al., 2000. Amino Acids 18:375-387) and act in concert with other proteins to contribute to the pathogenesis of anthrax. One reactive clone expressed one of the two paralogs in the genome of the sequenced B. anthracis Ames Strain (Read, T. D., et al., 2003. Nature 423:81-86), annotated as the immune inhibitor A metalloprotease (InhA), a secreted zinc-dependent metalloprotease that is also produced by other members of the B. cereus family, and is a component of the exosporium of the B. cereus spore (Charlton, S., et al., 1999. J. Appl. Microbiol. 87:241-245). InhA in B. anthracis may function in a manner similar to that in B. thuringiensis (Dalhammar, G. and H. Steiner. 1984. Eur. J. Biochem. 139:247-252) to inactivate bactericidal host proteins during early infection and facilitate bacterial survival within the host. InhA may, in fact, be part of a suite of proteins that contribute to protective immunity against anthrax. Also included in this group were two clones expressing putative membrane proteins of unknown function, which merit further evaluation as virulence determinants in view of their surface-location.

The screen identified two clones expressing proteins involved in sporulation. Identified proteins included a Spo0B-associated GTP binding protein of the Obg family and the RNA polymerase sigma-27 factor (SigK). Also identified were reactive clones expressing proteins involved in metabolism, such as the flavoprotein subunit of the membrane bound enzyme, succinate dehydrogenase (SdhA), an enzyme of the tricarboxylic acid cycle, which during aerobic growth converts succinate to fumarate. Fumarate reductase reportedly facilitates H. pylori colonization of the murine gastric mucosa, and hence has been proposed to be both a novel drug target and a putative vaccine candidate (Ge, Z., et al., 2000. Microb. Pathog 29:279-287). Of note, the B. subtilis SdhA has also been demonstrated to function as a fumarate reductase (Schnorpfeil, M., et al., 2001. Eur. J. Biochem. 268:3069-3074).

The screen identified several clones expressing proteins involved in the metabolism of macromolecules and energy. Also identified was an enzyme involved in DNA replication, recombination and repair called endonuclease IV. As an anthrax spore-surface protein, endonuclease IV may function in concert with other proteins to facilitate spore survival within macrophages. One clone expressed a putative glutathionylspermidine (GSP) synthase, an important intermediate in the biosynthesis of the antioxidant, tryptathione. A clone expressing the acetoin utilization protein, AcuC, which facilitates the utilization of the carbon storage compound, acetoin, via an undefined mechanism, was identified. Another clone contained an insert that included three genes. The first encoded an enzyme called acyl CoA dehydrogenase (ACDH) functioning in fatty acid and phospholipid metabolism and may be an important component of the stress response functioning in conjunction with other overlapping proteins to facilitate pathogen adaptation to the in vivo environment. The second gene on the insert encoded a cytoplasmic, conserved hypothetical protein, and the third gene encoded acetyl-CoA acetyltransferase, an enzyme involved in fatty acid and phospholipid metabolism. Of note, acetyl-CoA acetyltransferase is located on the anthrax spore-surface (Liu, H., et al., 2004. J. Bacteriol. 186:164-178). Also expressed from one of the clones in this group was an enolase functioning in glycolysis/gluconeogenesis. This enzyme is a component of the anthrax spore-surface (Liu, H., et al., 2004. J. Bacteriol. 186:164-178), and was recently reported to be a component of anthrax vaccine approved for human use in the UK (Whiting, G. C., et al., 2004. Vaccine 22:4245-4251).

Several reactive clones expressing proteins involved in amino acid biosynthesis were identified. Among the proteins expressed by such clones was methylribose kinase (MtnK) (identified twice), an enzyme that is unique to microbes (and plants) and plays a central role in the salvage of methionine (Sekowska, A., et al., 2001. BMC Microbiol 1:1570, Gianotti, A. J., et al., 1990. J. Biol. Chem. 265:831-837). Anthrax spore-associated MtnK may be a suitable target for the development of vaccines, drugs, and/or spore-inactivation agents. A functional ortholog of the autoinducer synthase, LuxS, responsible for the final step of AI-2 synthesis was recently reported in B. anthracis suggesting that this pathogen might also regulate density-dependent gene expression via AI-2 (Jones, M. B. and M. J. Blaser. 2003. Infect. Immun. 71:3914-3919). Another protein expressed from a reactive clone in this group was aspartate kinase I (DapG-1), which is involved in the first step of biosynthesis of diaminopimelate from L-aspartate. Diaminopimelate is an important constituent of both the peptidoglycan of vegetative cells and of the spore cortex peptidogylcan of Gram positive bacteria, especially in members of the genus Bacillus. Furthermore, dipicolinate, a by-product during diaminopimelate biosynthesis, is also a part of the spore, comprising as much as 10% of the dry spore weight (Chen, N. Y., et al., 1993. J. Biol. Chem. 268:9448-9465). Aspartokinases play a pivotal role in the biosynthesis of important structural components in diverse microbes.

Several reactive clones expressing proteins involved in the biosynthesis of nucleosides/nucleotides were identified. One such protein was the dihydroorotase, PyrC, which catalyzes one of the reactions in the biosynthesis of uridine monophosphate (UMP) from precursors such as aspartate and glutamine. It is likely that PyrC, as a spore component, functions in pyrimidine nucleotide synthesis during early infection before the elaboration of toxins and other degradative enzymes that cause cellular destruction, and rendering available uracil and other pyrimidine nucleotides to be utilized in the pyrimidine salvage pathway (the closely related B. subtilis possesses a pyrimidine salvage pathway, and hence it is likely that a similar pathway also exists in B. anthracis). PyrC may contribute to B. anthracis survival within the host.

Another protein involved in the synthesis of small molecules was thymidine kinase (Tdk), which functions in pyrimidine salvage (Agrawal, N., et al., 2004. Biochemistry 43:10295-10301). A spore-location alludes to a possible role in salvage of thymidine derivatives from host cells/tissues for DNA synthesis essential for multiplication of B. anthracis following spore-germination. The same cloned insert expressing Tdk also included part of the gene encoding the ribosomal protein L31, which is involved in the synthesis and modification of ribosomal proteins. A clone was also identified expressing the monofunctional, phosphoribosylamine-glycine ligase, PurD, (also called glycinamide ribonucleotide synthetase), an enzyme functioning in de novo purine ribonucleotide biosynthesis. Also in this group was a clone that expressed adenine phosphoribosyltransferase, an enzyme of the purine salvage pathway, which possibly performs a function analogous to the above enzymes of the pyrimidine salvage pathway.

A group of reactive clones expressed proteins involved in the biosynthesis of cofactors, prosthetic groups and carriers. Some expressed proteins functioning in thiamine biosynthesis: ThiC, ThiG, and ThiS. These enzymes function in the de novo synthesis of an important nutrient, namely thiamine (Zhang, Y., et al., 1997. J. Bacteriol. 179:3030-3035; Park, J. H., et al., 2003. Biochemistry 42:12430-12438), suggests a likely role in the in vivo survival of the pathogen. Coupled with the fact that the untranslated regions of mRNA specifying such enzymes contain a metabolite responsive genetic control element or “riboswitch” renders them attractive targets for drug development (Winkler, W., et al., 2002. Nature 419:952-956).

Several clones expressing enzymes involved in protein degradation were identified. One of these was a putative secreted aminopeptidase that belonged to the family of widely distributed metal-associated metalloproteases, which catalyze the removal of N-terminal amino acids from peptides and proteins. The region upstream of the gene encoding this protein has a binding site for PlcR, a pleiotropic regulator of extracellular virulence factors in closely related organisms such as B. thuringiensis (Agaisse, H., et al., 1999. Mol. Microbiol. 32:1043-1053; Read, T. D., et al., 2003. Nature 423:81-86). Although the PlcR homolog in B. anthracis is truncated due to a nonsense mutation, it has been hypothesized that alternative regulatory controls may allow for PlcR-regulated proteins to contribute to B. anthracis virulence (Read, T. D., et al., 2003. Nature 423:81-86). Also, the fact that aminopeptidases are present on the anthrax spore-surface (Liu, H., et al., 2004. J. Bacteriol. 186:164-178), and have been reported to play a role in pathogenesis, particularly of intracellular parasites (Morty, R. E. and J. Morehead. 2002. J. Biol. Chem. 277:26057-26065), suggests that they might play a role in the virulence of B. anthracis. Among other proteins expressed by reactive clones in this group was a peptidase T (PepT-2) (identified twice in this screen), a zinc metalloprotease and an amino tripeptidase, which removes the N-terminal amino acid residue from various tripeptides. Although the contribution of these proteins to the virulence of B. anthracis is unclear, it is of interest that PepT was one of the proteins highly expressed in E. coli K12 biofilms and during growth in preconditioned medium from the laboratory strain E. coli DH5α (Prigent-Combaret, C., et al., 1999. J. Bacteriol. 181:5993-6002), despite the fact that cell-to-cell signaling via acyl homoserine lactone (acyl-HSL) molecules is yet to be demonstrated in Gram positive bacteria, including B. anthracis (Bassler, B. L. 2002. Cell 109:421-424). Another spore-associated protein was a putative prolyl oligopeptidase family protein. Because members, such as dipeptidyl peptidase IV, have been implicated in the virulence of certain bacterial pathogens (Yagishita, H., et al., 2001. Infect. Immun. 69:7159-7161), this protein warrants further study regarding its contribution to the pathogenicity of B. anthracis.

The screen identified two clones expressing regulatory proteins. One of these was a sensory box histidine kinase component of an unknown two-component regulatory system. Although speculative, the fact that sensor kinases sense and transduce signals from the environment to cognate response regulator components to influence gene expression (James A. Hoch and Thomas Silhavy (eds.). 1995. ASM Press, Washington, D.C.), renders it plausible that a spore-surface sensor kinase might be involved in sensing the environment within the macrophage and transducing a signal via its response regulator to affect expression of genes involved in early infection. The other protein identified as part of this group was a LysR-type transcriptional regulator, which in a variety of pathogens is reportedly involved in the positive regulation of diverse classes of genes, including those encoding virulence factors (Schell, M. A. 1993. Annu. Rev. Microbiol. 47:597-626). The screen identified another LysR-type transcriptional regulator encoded on the same insert that also encoded a transporter of the EamA family. The finding that LysR-type regulators were associated with the anthrax spore was not unexpected since such proteins have been identified as constituents of the anthrax spore-surface (Liu, H., et al., 2004. J. Bacteriol. 186:164-178); however, the roles played by these proteins in this location is yet to be defined.

Two reactive clones expressing proteins involved in cellular processes were identified. One of these was an uncharacterized catalase that may be part of the oxidative stress response protecting germinating spores against the lethal effects of H₂O₂, especially within phagocytic cells. It is plausible that this uncharacterized, spore-associated catalase might act in conjunction with KatX, a catalase present in B. subtilis spores (Bagyan, I., et al., 1998. J. Bacteriol. 180:2057-2062), and with other spore-coat resident enzymes such as superoxide dismutase, to dissipate H₂O₂ and protect germinating spores against oxidative damage. Other proteins included within this group included a cell division initiation protein, DivIVA, which functions in the proper positioning of the septum during cell-division and also promotes asymmetric septation, an essential prerequisite for sporulation (Cha, J. H. and G. C. Stewart. 1997. J. Bacteriol. 179:1671-1683).

The screen identified a group of clones that expressed proteins of unknown function. Included among these was an acyl transferase of the Gcn5-related acyl transferase (GNAT) superfamily, the members of which are widely distributed in nature and use acyl CoAs to acylate their respective substrates. Interestingly, a paralog in the sequenced B. anthracis Ames strain (BA1085), which is also an acyl transferase of the Gcn5-related acyl transferase (GNAT) superfamily, has been reported to contain the upstream binding motif for the pleiotropic positive regulator of extracellular virulence factor gene expression, PlcR (Read, T. D., et al, 2003. Nature 423:81-86). Also identified by the screen was a carboxyl transferase domain protein, which catalyzes the transfer of a carboxyl group from biotin to an acceptor acyl-CoA, a chlorohydrolase family protein (a family of enzymes that are a large metal dependent hydrolase superfamily); a hydrolase of the carbon-nitrogen hydrolase family functioning in nitrogen metabolism; and an aminotransferase, which catalyzes the transfer of an amino group to a cognate acceptor. Among this group of clones was one that expressed a hydrolase of the alpha/beta fold family with aminopeptidase activity, which was previously reported to be a component of the exosporium of the anthrax spore (Liu, H., et al., 2004. J. Bacteriol. 186:164-178). Also identified was a protein encoded by vrrA (variable region with repetitive sequence), which encodes a 30-kDa protein in the Sterne strain but encodes truncated proteins in the Ames strain and Vollum strain, due to a single nucleotide and a 24-bp deletion, respectively (Andersen, G. L., et al., 1996. J. Bacteriol. 178:377-384). Despite this, the fact the amino acid sequence of VrrA of B. anthracis Sterne differs from that of the closely related B. cereus and B. mycoides at 61 different positions (Andersen, G. L., et al., 1996. J. Bacteriol. 178:377-384), and also the fact that this protein was a target of the AVA-induced immune response in humans suggests that VrrA could be a potential virulence determinant of B. anthracis. Finally, the screen identified a clone expressing a 15.2-kDa hypothetical protein (BA5515) of unknown function. This hydrophilic spore-associated protein was encoded by a 360 bp gene that was present in the sequenced genomes of both B. anthracis Ames and Sterne strains, but not in any of the heretofore-sequenced genomes of close relatives as evidenced by BLAST analysis. Also, no significant homology to other database entries was detected.

In summary, 69 clones expressing anthrax spore-associated proteins targeted by AVA-induced immunity were identified. Positive clones expressed proteins previously identified by other methods as constituents of the anthrax spore-surface, proteins highly expressed during spore germination, proteins that were orthologs of drug targets and virulence determinants of diverse pathogens, and several proteins of unknown function. Of note, the majority of proteins identified by this screen were not spore structural proteins but, rather, proteins expressed during vegetative growth. It is possible that, when on the spore-surface, proteins expressed during vegetative growth-phase that are also spore-associated take on completely different roles than those ascribed to them during vegetative growth, such as those that help establish early infection and spore germination. Such disparate roles for the same protein at different cellular locations have been described previously in other pathogens (Heithoff, D. M., et al, 1999. J. Bacteriol. 181:799-807).

The functions ascribed above to the targets of AVA-induced immunity are putative and not be construed as limiting. More extensive studies to determine the definitive roles of these SA-proteins have commenced. Because the proteins identified in this study are associated with the infective form of Bacillus anthracis (which is likely to interact first with components of the host immune system), and because the expression of a subset of SA-proteins is reportedly increased during spore-germination (Huang, C. M., et al., 2004. Proteomics 4:2653-2661), various approaches are being employed to identify SA-proteins operating during early infection with anthrax spores. Furthermore, because a subset of SA-proteins identified herein were either orthologs of proteins of diverse pathogens under investigation as drug targets, or virulence determinants of both Gram positive and Gram negative bacteria, deletions are being generated of genes encoding selected SA-proteins in various B. anthracis strains to determine the contribution of such proteins to virulence of the pathogen using relevant animal models. The proteins identified by these studies are then further evaluated as an optimally delivered, PA-based vaccine, for protection of appropriate animal models against a challenge with virulent B. anthracis strains. Results of such studies facilitate the development of defined, non-reactogenic anthrax vaccines. In addition, because these proteins are part of the protein repertoire of the spore-surface, a subset of which have been reported to be highly expressed during germination (Huang, C. M., et al., 2004. Proteomics 4:2653-2661), the above-delineated studies help identify SA-proteins with potential for the development of drugs or spore-inactivation strategies. Finally, because of the spore-surface localization of SA-proteins and accessibility to ligands, such as antibodies, experiments have been initiated that are geared toward the identification of B. anthracis-specific domains within SA-proteins, as well as toward confirmation of spore-surface-localization of such domains for the development of assays for spore-detection.

Although the foregoing invention has been described in some detail by way of illustration and example for purposes of clarity and understanding, it will be apparent to those skilled in the art that certain changes and modifications can be practiced. Therefore, the description and examples should not be construed as limiting the scope of the invention, which is delineated by the appended numbered claims.

### Example 2

This example will prophetically describe the further evaluation of identified anthrax spore associated proteins.

Identification of individual spore-associated proteins that contribute to protective immunity, and optimization of formulation and delivery of such proteins to the immune system, can result in the development of more efficacious second and third generation multivalent anthrax vaccines. Because protective efficacy of a multivalent vaccine comprising these proteins cannot be directly studied in humans due to ethical reasons, the vaccine potential of these proteins can be addressed in animal models (in accordance with the recent “animal rule” see Food and Drug Administration “New drug and biological drug products: evidence needed to demonstrate effectiveness of new drugs when human efficacy studies are not ethical or feasible.” Fed Regist 2002; 67:37988-98] criteria proposed by the US Food and Drug Administration [FDA] for demonstrating vaccine effectiveness in situations that preclude human volunteer challenge studies by allowing reasonably well-understood models to substitute for human studies).

The following scheme for further evaluation of the vaccine potential of identified proteins will be accomplished using well-established experimental methods:

Step 1. Each protein will be purified to homogeneity/near homogeneity using defined sequential chromatographic protein purification techniques. Proteins that are difficult to purify will be subjected to bioinformatics to select hydrophilic, surface-exposed domains (most likely to be recognized by the immune system), which will then be chemically synthesized.

Step 2. Step 1 will be followed by a preliminary evaluation of the vaccine potential of each protein using A/J mice (a mouse strain that is highly susceptible to the attenuated, experimental Bacillus anthracis Sterne strain which can be used in these experiments). Defined amounts of each purified protein will be injected intraperitoneally into groups of A/J mice without and with appropriate adjuvants on day 0, and boosted again on day 14. Immunized mice will then be challenged on day 28 with a defined number B. anthracis Sterne (the anthrax vaccine approved for human-use in the USA is derived from the culture supernatant of a related strain) with 10×LD₅₀ spores via intranasal instillation or aerosol. The “time to death” will be noted for each group and compared with that for unimmunized mice, and survival curves will be plotted. Spore-associated proteins that significantly increases the time to death/completely protect mice are vaccine candidates that warrant further study.

Step 3. The next batch of experiments will involve the evaluation of the vaccine candidate proteins as a multivalent experimental vaccine administered via transcutaneous immunization (TCI). Transcutaneous immunization [TCI] is a needle-free method of immunization that involves application of protein antigens co-administered with an adjuvant on intact skin, resulting in the development of robust systemic and mucosal immune responses both against the antigens, as well as the adjuvant. For TCI, vaccine candidate proteins will be pooled, and used to immunize A/J mice transcutaneously along with cholera toxin (CT) as an adjuvant. The experimental vaccine will contain 50 μg of each protein will be administered with 50 μg of adjuvant and without or with 50 μg of protective antigen (PA), the nontoxic receptor binding moiety of anthrax toxins, which is the principal component of AVA. These experiments will allow a direct comparison and evaluation of the efficacy of the experimental vaccine with and without PA, and hence might dictate the use of the multivalent experimental vaccine either as a more efficacious second generation (with PA) or a novel third generation anthrax vaccine (without PA). Several groups of A/J mice will be administered a primary immunization (day 0) or a primary and a booster immunization (day 0 and day 14) via TCI with the respective experimental vaccine formulation. Mice will then be challenged as described above on day 28. Efficacy will be assessed by the ability of the experimental vaccine to protect A/J mice against a lethal challenge with B. anthracis, compared with that of a control group of unimmunized A/J mice. The duration of protective immunity will then be assessed by challenging A/J mice at 28-day intervals (starting from day 28 to day 168).

Step 4. A parallel set of identical experiments will be performed using CpG oligonucleotides as an adjuvant instead of CT.

Step 5. The above set of experiments should yield information leading to optimization of the immunization regimen, formulation of the multivalent experimental vaccine and the best adjuvant for induction of long-lasting protective immunity. To confirm efficacy, the multivalent experimental vaccine will evaluated in another mammalian species, namely, rabbits, via TCI using the identical experimental protocol described above. Immunized rabbits will be challenged as outlined above using fully virulent strains of B. anthracis.

Step 6. Similar experiments will also be performed in both mice and rabbits, in which genes encoding spore-associated proteins will be cloned into suitable plasmid DNA vectors and administered as a multivalent genetic (DNA) vaccine.

SEQUENCES

BAS0087
Accession No. NC_005945, REGION: 97324 . . . 98781
Bacillus anthracis str. Sterne, complete genome.
Bases 1 to 1458
ORIGIN

SEQ ID NO: 1

| | |
|---|---|
| 1 | atggaaaagc aagtgagagt gcgctatgcg ccaagtccaa caggacactt acatatcgga |
| 61 | aatgcgcgta cggcattatt taattattta tttgctcgtc atcaagatgg taagtttatt |
| 121 | attcgtattg aagatactga tgtaaaacgt aatgttgctg gtggagaaga aagccaatta |
| 181 | aaatacttga aatggctcgg tatggactgg gatgaaggtg ttgatgttgg tggtgaattt |
| 241 | ggaccatatc gtcaaacaga gcgtttagat atttataaaa agttatatga agatttatta |
| 301 | gagcgtggtt tagcttacaa atgttatatg acagaagaag agctagaggc agaacgcgaa |
| 361 | gggcaaattg ctcgtggtga aacacctcgt tacgcaggca accaccgtga tttaactgaa |
| 421 | gcgcaagtga aagaatttga agctgaggga cgtattccaa gtattcgttt ccgcgtacca |
| 481 | gctgaccgtg attacacatt taaagatatt gtaaaagatg aagttgcatt ccattcaaat |
| 541 | gatttcggtg attttgttat cgtgaaaaaa gatgggattc caacttataa ctttgcagta |
| 601 | gcagtagatg atcacttaat ggaaattaca cacgtacttc gtggtgatga ccatatttca |
| 661 | aacacaccaa aacaaatgat gatttatgaa gctttcggtt gggatattcc gcaattcggt |
| 721 | catatgactt taattgtaaa tgaaagccgt aaaaaattaa gtaagcgtga tgaatctatt |
| 781 | attcaattta ttgagcaata taaagagctt ggatatcttc cagaagcaat ctttaacttt |
| 841 | attgcactac taggttggtc gccagtagga gaagaagaaa tcttctctca agaagagttt |
| 901 | atcaaaatgt ttgatgcagc tcgtttatca aaatcacctg cattatttga ttctcaaaaa |
| 961 | ctaaaatgga tgaacaacca atatatgaaa aagcaagatt tagatacggt ggtagaatta |
| 1021 | agcttaccgc atttagtgaa ggctggacgt ataggtgaaa ctttaagtga acaagaacaa |
| 1081 | gcttggattc gtgatgtaat tgcgttatat catgaacaaa tgagctttgg agctgaaatt |
| 1141 | gtagagcttt ctgaaatgtt cttcaaagat cacgttgatt atgaagaaga aggacaagaa |
| 1201 | gtattaaaag gtgaacaagt accagaagta cttcgtgcat ttgctggtca agtagaagca |
| 1261 | ctagaagcta tggaaccggc agcaattaag gcggctatta aagcggttca aaaggaaaca |
| 1321 | ggtcataaag gtaaaaactt atttatgcca atccgtgttg caactactgg tcaaacacat |
| 1381 | ggcccagagc ttcctaatgc tattgcactt cttggaaaag aaaaagtttt aaatcgtctt |
| 1441 | caaaaagtaa tcggttaa |

BAS0087
Accession No. NC_005945
Bacillus anthracis str. Sterne, complete genome.

SEQ ID NO: 2

MEKQVRVRYAPSPTGHLHIGNARTALFNYLFARHQDGKFIIRIEDTDVKRNVAGGE
ESQLKYLKWLGMDWDEGVDVGGEFGPYRQTERLDIYKKLYEDLLERGLAYKCYM
TEEELEAEREGQIARGETPRYAGNHRDLTEAQVKEFEAEGRIPSIRFRVPADRDYTF
KDIVKDEVAFHSNDFGDFVIVKKDGIPTYNFAVAVDDHLMEITHVLRGDDHISNTP
KQMMIYEAFGWDIPQFGHMTLIVNESRKKLSKRDESIIQFIEQYKELGYLPEAIFNFI
ALLGWSPVGEEEIFSQEEFIKMFDAARLSKSPALFDSQKLKWMNNQYMKKQDLDT
VVELSLPHLVKAGRIGETLSEQEQAWIRDVIALYHEQMSFGAEIVELSEMFFKDHVD
YEEEGQEVLKGEQVPEVLRAFAGQVEALEAMEPAAIKAAIKAVQKETGHKGKNLF
MPIRVATTGQTHGPELPNAIALLGKEKVLNRLQKVIG
BAS3884
Accession No. NC_005945, REGION: 3833569 . . . 3834123
Bacillus anthracis str. Sterne, complete genome.
Bases 1 to 555
ORIGIN

SEQ ID NO: 3

| | |
|---|---|
| 1 | atgcttacaa tgaaagatgt aattcgcgaa ggagatccta ttttgcgaaa cgttgcagaa |
| 61 | gaggtagtaa taccagcgag cgaagaagat acaaataccc ttaaagaaat gattgaattt |
| 121 | gtaataaata gccaagatcc tgaaatggct gaaaaatata gtttacgccc tggaatcgga |
| 181 | ttagcggctc cgcaaatcgg tatttcaaag aaaatgattg cagttcacgt aacagatacg |
| 241 | gacggtacgt tatatagtca tgcattattc aatccaaaaa tcattagcca ttctgttgaa |
| 301 | cgtacatatt tacaaagtgg tgaaggctgt ctatcagtag accgtgaagt acctggttat |
| 361 | gtacctcgtt atacaagaat tacagtgaaa gcaacttcta tcaacggcga agaagtaaaa |
| 421 | ttacgtttaa aaggtttacc agcaattgta ttccaacatg aaattgacca tttaaatggt |
| 481 | gttatgttct atgaccatat taataaagaa aatccatttg ctgctcctga cggttcaaaa |
| 541 | cctctggagc gataa |

BAS3884
Accession No. NC_005945
Bacillus anthracis str. Sterne, complete genome.

SEQ ID NO: 4

MLTMKDVIREGDPILRNVAEEVVIPASEEDTNTLKEMIEFVINSQDPEMAEKYSLRP
GIGLAAPQIGISKKMIAVHVTDTDGTLYSHALFNPKIISHSVERTYLQSGEGCLSVDR
EVPGYVPRYTRITVKATSINGEEVKLRLKGLPAIVFQHEIDHLNGVMFYDHINKENP
FAAPDGSKPLER
BAS4955
Accession No. NC_005945, REGION: complement(4836199 . . . 4836666)
Bacillus anthracis str. Sterne, complete genome.
Bases 1 to 468
ORIGIN

SEQ ID NO: 5

| | |
|---|---|
| 1 | atgccaaaag gttcaggtaa ggttattgca caaaataaaa aagcatttca tgattatttc |
| 61 | atcgaagaaa catacgaagc agggcttgtc cttcaaggaa cggaaattaa gtcgattcgc |
| 121 | gctggacgcg tgaacttgaa agatgcgttt gcacgtgtac ataatggtga agtatgggtt |
| 181 | cataatatgc atattagtac gtacgaacaa gggaatcgtt tcaaccacga tccgcttcgc |
| 241 | acgagaaagt tacttcttca taaaaaagaa attgagaagt tagcgggtgc ttcaaaagaa |
| 301 | acaggatatg cactagttcc agttagaatc tatttgaaaa atggatttgc gaaaatggca |
| 361 | cttggtttag caaaaggtaa gaaacaatac gataaacgtc acgatttaaa agagaaagaa |
| 421 | gctaaacgtg aaattgcacg cgcgttccgt gatcgccaaa agatgtaa |

BAS4955
Accession No. NC_005945
Bacillus anthracis str. Sterne, complete genome.

SEQ ID NO: 6

MPKGSGKVIAQNKKAFHDYFIEETYEAGLVLQGTEIKSIRAGRVNLKDAFARVHNG
EVWVHNMHISTYEQGNRFNHDPLRTRKLLLHKKEIEKLAGASKETGYALVPVRIYL
KNGFAKMALGLAKGKKQYDKRHDLKEKEAKREIARAFRDRQKM
BAS4956
Accession No. NC_005945, REGION: complement(4836915 . . . 4839341)
Bacillus anthracis str. Sterne, complete genome.
Bases 1 to 2427
ORIGIN

SEQ ID NO: 7

| | |
|---|---|
| 1 | ttggaagaaa tcatacaaga acatattgat aagttgttat tatttatgag agaagaagcg |
| 61 | tataaaccgc taacgataca agagttagaa gaggcatttg ggattgaagg ttccgagggc |
| 121 | tttaaagatt tcgtaaaggc acttgtaacg atggaagaaa agggactcgt tattcgtact |
| 181 | cgtagcaacc gttacggtct tcctgaaaag atgaatttaa tacgtggtaa gttaattgga |
| 241 | catgcacgtg gttttgcatt tgttgtacca gacgagaaga aaacgggaga cgatgatctt |
| 301 | ttcatcccac ctacagaatt aaacggtgcg cttcatggtg atacagtatt agcacgcctt |
| 361 | agttcccaat cgagtggttc gcgtcaagaa ggttctattg tacgcatttt agaacgtgga |
| 421 | acgaaagaac tagttggtac atatacagaa tcgaaaaact ttggatttgt tatacctgac |
| 481 | aataagcgct ggacgagtga cattttcgta ttgaaaagtg catcaatggg tgctgtagaa |
| 541 | ggtcataaag tagttgtgaa aattacgagc tatccagaga atcgtttaag tgcagaaggt |
| 601 | gaagttattc aaattctagg tcataaaaat gacccaggag tagatatttt atctgttatt |
| 661 | cataaacatc atttaccttt agcattccca gaggaagtga tggaacacgc aaacagtgta |
| 721 | ccagaaacga tttcagagga agatttaaaa gatcgccgtg acctgcgtga ccaaatgatc |
| 781 | gtaacaattg acggtgcaga cgcaaaagat ttagatgacg ctgttacagt aacaaagctt |
| 841 | gagaacggta actataaact tggcgttcat attgcggatg taagtcatta cgttcaagaa |
| 901 | ggttctccaa ttgatgtaga agcagcggag agagcgacga gtgtatatct tgttgaccgt |
| 961 | gtaattccaa tgatcccgca tcgtctatct aacggtattt gttcattaaa tccgaaagta |
| 1021 | gaccgtctga cgttatcttg tgaaatggaa attaacaatt taggtgacgt tgtaaaacac |
| 1081 | gagattttcc aaagtgtgat taaaacgaca gagcgtatga cgtatgctga cgtaagaagc |
| 1141 | attttagaag atgaggacga agaattaatg aaacgctatg agccgctcgt accgatgttt |
| 1201 | aaagagatgg ggcaattagc acaaatttta cgtgaaaaac gtatgcgccg cggggcaatc |
| 1261 | gactttgact ttaaagaagc gaaagtatta gtagatgaag aaggaaaacc gacagatgtt |
| 1321 | gttatgcgtg atcgttctgt atcagagaag ttaattgaag aatttatgct tgttgcaaac |
| 1381 | gaaacagtag cagagcactt ccactggatg aacgtaccat tcatgtaccg tgtccatgaa |
| 1441 | gatccgaaag aagataagtt agagcgtttc ttcgagtttg taacgaactt cggatatgca |
| 1501 | gtaaaaggac gtgcgaatga agtacatcct cgcgcgctac aacaaattct tgaaatggtt |
| 1561 | caaggacagc cagaagaagt agtaatctca acagttatgc ttcgttctat gaagcaagca |
| 1621 | cgttacgatg cagatagctt aggacatttc ggtttatcaa ctgagttcta cacacatttc |
| 1681 | acatcgccaa ttcgtcgtta cccagatacg attgttcata gattaattcg tgaatacatc |
| 1741 | attaacggta aagtcgacaa tgaaacacaa gcaaagtggc gtgaaaaatt acctgagatt |
| 1801 | gcagagcact cttctaatat ggagcgtcgt gctgttgaag cagaacgtga aacagatgag |
| 1861 | ctgaaaaaag cagaatatat gcttgataag attggcgaag agtatgacgg tatgattagc |
| 1921 | tctgtaacaa acttcggttt attcgtagag cttccaaata caattgaagg tcttgtacac |
| 1981 | gttagctact taacggatga ttactaccgt tacgatgagc agcatttcgc aatgatcgga |
| 2041 | gaacgtacag gtaacgtatt ccgcatcggt gacgaaatta caattcgtgt tattaatgta |
| 2101 | aacaaagacg agcgtgcaat cgactttgaa atcgttggca tgaaaggtac acctcgtcgt |
| 2161 | aagttcaaag accgcccagt cgttattgaa cagccaagaa caggtagaaa gaaacgcggt |
| 2221 | ggacgtagcg agcgcagtaa tgagcgcggc ggagaacgtg gcacaggaag aaaatttgac |
| 2281 | cgtggtggca aagggaaagg aagaggatct gcatccgcat ctacgtccgc tagecagcca |
| 2341 | gggaaaaaag atggtaacgg caagaagaaa aaagcattct tcgaaaacgt accaggattc |
| 2401 | aagaagaaaa agaaaaagcg taagtaa |

BAS4956
Accession No. NC_005945
Bacillus anthracis str. Sterne, complete genome.

SEQ ID NO: 8

MEEIIQEHIDKLLLFMREEAYKPLTIQELEEAFGIEGSEGFKDFVKALVTMEEKGLVI
RTRSNRYGLPEKMNLIRGKLIGHARGFAFVVPDEKKTGDDDLFIPPTELNGALHGDT
VLARLSSQSSGSRQEGSIVRILERGTKELVGTYTESKNFGFVIPDNKRWTSDIFVLKS
ASMGAVEGHKVVVKITSYPENRLSAEGEVIQILGHKNDPGVDILSVIHKHHLPLAFP
EEVMEHANSVPETISEEDLKDRRDLRDQMIVTIDGADAKDLDDAVTVTKLENGNY
KLGVHIADVSHYVQEGSPIDVEAAERATSVYLVDRVIPMIPHRLSNGICSLNPKVDR
LTLSCEMEINNLGDVVKHEIFQSVIKTTERMTYADVRSILEDEDEELMKRYEPLVPM
FKEMGQLAQILREKRMRRGAIDFDFKEAKVLVDEEGKPTDVVMRDRSVSEKLIEEF
MLVANETVAEHFHWMNVPFMYRVHEDPKEDKLERFFEFVTNFGYAVKGRANEVH
PRALQQILEMVQGQPEEVVISTVMLRSMKQARYDADSLGHFGLSTEFYTHFTSPIRR
YPDTIVHRLIREYIINGKVDNETQAKWREKLPEIAEHSSNMERRAVEAERETDELKK
AEYMLDKIGEEYDGMISSVTNFGLFVELPNTIEGLVHVSYLTDDYYRYDEQHFAMI
GERTGNVFRIGDEITIRVINVNKDERAIDFEIVGMKGTPRRKFKDRPVVIEQPRTGRK
KRGGRSERSNERGGERGTGRKFDRGGKGKGRGSASASTSASQPGKKDGNGKKKK
AFFENVPGFKKKKKKRK
BAS5178
Accession No. NC_005945, REGION: complement(5057771 . . . 5058847)
Bacillus anthracis str. Sterne, complete genome.
Bases 1 to 1077
ORIGIN

SEQ ID NO: 9

| | |
|---|---|
| 1 | gtgaatgatg tgttagatcg tttgcaagct gtagaaaatc gttatgagaa gttaaatgaa |
| 61 | ttgctaagcg acccagcaat tattagtgat tcaaataagc ttcgtgaata ttcaaaggaa |
| 121 | cagtctgata tacaggaaac ggtagaggtg tatcgtgagt ataaggatgt tcgtgagcaa |
| 181 | ttaaaagatg cgaaagcaat gttagaagat aagttagacg cagaaatgcg tgaaatggta |
| 241 | aaagaagagg tttctgagct agaatcacaa gaaaaaacat tatcagagcg tctgaaaatt |
| 301 | ttacttgtac caaaagatcc taacgatgat aagaacgtta tcgttgaggt tcgtggagct |
| 361 | gccggtggtg acgaggctgc tttatttgct ggtgatttat accgtatgta tagccgttac |
| 421 | gctgaggtac aaggttggaa aacggagatt atcgaggcta gctatacaga gttaggtgga |
| 481 | tataaagaga ttatctttat gattaacggt aaaggtgctt tcgcgaagtt gaaatttgag |
| 541 | aatggcgctc accgtgtaca acgtgttcct gaaacggaat ctggtggacg tattcataca |
| 601 | tctacagcaa ctgtagctgt attaccagag gcagaagaag tagaaattga tattcatgag |
| 661 | aaagatgttc gtgttgatac attcgcttct agtggacctg gtggacagag cgttaataca |
| 721 | acgatgtcag cggtacgttt aacgcattta ccgactggtg tagttgtatc gtgtcaggat |
| 781 | gagaaatcac aaattaagaa taaagaaaaa gcgatgaaag tattacgcgc acgtgtttat |
| 841 | gataagttta gacaagaagc acaagctgag tatgatcaaa accgtaaaca agctgttggt |
| 901 | acgggtgatc gttcagagcg tattcgtacg tataacttcc cgcaaaaccg tgttacagac |
| 961 | catcgaatcg gtttaacgat tcaaaagcta gatcaaatct tacaaggtaa gttagatgat |
| 1021 | ttcatcaatg ccttagtgat ggaagatcag gctcaaagga tggaggcagc tgagtaa |

BAS5178
Accession No. NC_005945
Bacillus anthracis str. Sterne, complete genome.

SEQ ID NO: 10

MNDVLDRLQAVENRYEKLNELLSDPAIISDSNKLREYSKEQSDIQETVEVYREYKD
VREQLKDAKAMLEDKLDAEMREMVKEEVSELESQEKTLSERLKIILLVPKDPNDDK
NVIVEVRGAAGGDEAALFAGDLYRMYSRYAEVQGWKTEIIEASYTELGGYKEIIFM
INGKGAFAKLKFENGAHRVQRVPETESGGRIHTSTATVAVLPEAEEVEIDIHEKDVR
VDTFASSGPGGQSVNTTMSAVRLTHLPTGVVVSCQDEKSQIKNKEKAMKVLRARV
YDKFRQEAQAEYDQNRKQAVGTGDRSERIRTYNFPQNRVTDHRIGLTIQKLDQILQ
GKLDDFINALVMEDQAQRMEAAE
BAS0016
Accession No. NC_005945, REGION: 21870 . . . 22421
Bacillus anthracis str. Sterne, complete genome.
Bases 1 to 552
ORIGIN

SEQ ID NO: 11

| | |
|---|---|
| 1 | ttgacaacag cgcatataag tggagattgg agaactcgtt accaacacgt aacgagtttt |
| 61 | tattttaaaa ccaatcataa agtatggtgt aaggatataa tgtccttatt acacactcat |
| 121 | attccgatga ttatcttcat agatatgaaa ggagtcaaca tggatcttat tatacaaacg |
| 181 | tttcctttag atggaaaaac tttatattat gtacaatgtc ctgtctgtaa gaacaataga |
| 241 | attttaaaca gtggtgcaaa tgtatcacgc attattagcg atgatacatt ccgtaaactt |
| 301 | tgtggttgca cttgtgacgt aaagcaaact gcaacaaaag tagaggcacc aaaaaaagtt |
| 361 | aaaaaagaag ctgtaaagaa agaagcagct ccaaaacgta caggtaaagt attaacagca |
| 421 | gtaattaacg ggaaagaaat gactgttaaa gagattgctg aggcgtacga tattagtaca |
| 481 | agtactgttc gtcagcgtat taacgctgga aaatctgaga gtgaaattat tgctccgaca |
| 541 | aagaagaagt aa |

BAS0016
Accession No. NC_005945
Bacillus anthracis str. Sterne, complete genome.

SEQ ID NO: 12

MTTAHISGDWRTRYQHVTSFYFKTNHKVWCKDIMSLLHTHIPMIIFIDMKGVNMDL
IIQTFPLDGKTLYYVQCPVCKNNRILNSGANVSRIISDDTFRKLCGCTCDVKQTATK
VEAPKKVKKEAVKKEAAPKRTGKVLTAVINGKEMTVKEIAEAYDISTSTVRQRINA
GKSESEIIAPTKKK
BAS0015
Accession No. NC_005945 REGION: 20339 . . . 21613
Bacillus anthracis str. Sterne, complete genome.
Bases 1 to 1275
ORIGIN

SEQ ID NO: 13

| | |
|---|---|
| 1 | atgcttgata ttaaattttt acgtacaaat tttgaagaag taaaagcaaa gttacagcat |
| 61 | agaggcgaag atttaactga ttttggtcgc tttgaagaat tggatacgag aagaagagaa |
| 121 | ctacttgttc aaacagagga actaaaaagt aaacgtaacg aagtatctca acaaatctct |
| 181 | gtattgaagc gcgaaaagaa agatgcagaa gctctaattc tagaaatgcg tgaagttgga |
| 241 | gaaaaagtaa aagatcttga taatgaactt cgtacagttg aagaagattt agaaagattg |
| 301 | atgttatcta ttccaaatat ccctcacgaa tctgctccag ttggtgaaac agaggatgat |
| 361 | aatgtagtag ctcgtacttg gggagaagtg aaagaatttg cttttgaacc aaaaccacat |
| 421 | tgggatcttg ctacagattt aggaatctta gattttgagc gtgctggaaa agtaacagga |
| 481 | agccgcttcg tattctacaa aggtgctggc gcaagattag agcgtgcttt aattagcttt |
| 541 | atgcttgatc ttcatactga tgagcatgga tatgaagaag tattacctcc gtacatggta |
| 601 | aaccgtgcaa gcatgacagg gacaggacaa cttccgaagt ttgaagaaga tgcattccgt |
| 661 | attgaaagtg aagactactt cttaattcca acagctgaag tacctgtaac aaatatgcac |
| 721 | cgtgatgaaa tcttaaataa agagcaatta cctataagat atgctgcatt tagctcttgt |
| 781 | ttccgttctg aagcaggttc agctggccgt gatacacgtg gtttaattcg tcagcatcag |
| 841 | ttcaataaag tagagcttgt aaagttcgta aaaccagaag attcttacga agagttagaa |
| 901 | aaactaacaa atgatgcaga acgcgtgtta caattattag agttgccata tcgcgttatg |
| 961 | agcatgtgca caggcgattt aggatttaca gcagcgaaga aatacgatat cgaagtatgg |
| 1021 | attccaagct atggcacata tcgtgaaatc tcttcttgta gtaatttcga ggctttccaa |
| 1081 | gcgagacgtg caaatatccg tttccgtcgt gagccaaacg gcaaaccaga acatgttcat |
| 1141 | acattaaatg gatctggtct tgcaattgga cgtacggtag cagctatttt agagaactac |
| 1201 | caacaagaag atggtacaat tataattcca gaagttcttc gcccttatat gggaggaaaa |
| 1261 | acagttatta agtaa |

BAS0015
Accession No. NC_005945
Bacillus anthracis str. Sterne, complete genome.

SEQ ID NO: 14

MLDIKFLRTNFEEVKAKLQHRGEDLTDFGRFEELDTRRRELLVQTEELKSKRNEVS
QQISVLKREKKDAEALILEMREVGEKVKDLDNELRTVEEDLERLMLSIPNIPHESAP
VGETEDDNVVARTWGEVKEFAFEPKPHWDLATDLGILDFERAGKVTGSRFVFYKG
AGARLERALISFMLDLHTDEHGYEEVLPPYMVNRASMTGTGQLPKFEEDAFRIESE
DYFLIPTAEVPVTNMHRDEILNKEQLPIRYAAFSSCFRSEAGSAGRDTRGLIRQHQFN
KVELVKFVKPEDSYEELEKLTNDAERVLQLLELPYRVMSMCTGDLGFTAAKKYDIE
VWIPSYGTYREISSCSNFEAFQARRANIRFRREPNGKPEHVHTLNGSGLAIGRTVAAI
LENYQQEDGTIIIPEVLRPYMGGKTVIK
BAS0206
Accession No. NC_005945, REGION: 205187 . . . 206140
Bacillus anthracis str. Sterne, complete genome.
Bases 1 to 954
ORIGIN

SEQ ID NO: 15

| | |
|---|---|
| 1 | atgataatag gagctttagc atgtttgatt gcaagtatgt catggggagc gatgtttcca |
| 61 | gttgctgatc atgcgttaga atacatagat ccgttttatt tttcgcttat tcgctatgga |
| 121 | gcggtggcga taatgctgat tatattgttg ttaatgaaag aagggaaaca ggcatttcgt |
| 181 | ttagaaggaa gaggaaagtt actcgtcttt ttcggaacga tggcgtttac tgtatataat |
| 241 | gtacttatat ttttaggtca aatgttaatg ggaaaatcag gcgtgatggt agcctccatt |
| 301 | atggaagcac ttatgccgat gatttctatt tgtatcctat ggggatataa gcatgtaaaa |
| 361 | ccgaaaaagt atatgataac gagcatgctt atcgctttta taggggctgt atttgttatt |
| 421 | acaaaaggtg atatgagttt ctttttaaca ttgaaagata acatgttttc gctagcatgt |
| 481 | atatttgttg gagttgtggg ctgggttatt tatacgatgg gtggtcaaac gtgtagcgat |
| 541 | tggtcaacat tacgttattc tacgttgacg tgtgtattcg gtacgactgt tacaggaatt |
| 601 | ataacgataa ttataacggc gtttggatat gtttcagttc cgaatatggg aacgatttct |
| 661 | attgtgaaat acgatttatt atttatgatg acattaccag gaatagtagc gttactagct |
| 721 | tggaactatg gtgtgaaaat tttatcgtcc attaatggga ttttatttat taattttgta |
| 781 | cccattacaa ctttagttat tatgatgatg caaggatatc aaataacaat gtttgatatt |
| 841 | gtagggactt tacttgttat tgcagcactt attcgtaata atgtttgtca gaggaaagaa |
| 901 | gaaaatatca acaagagaat tttagaaaaa gagcaattac gtcaagctgt ttaa |

BAS0206
Accession No. NC_005945
Bacillus anthracis str. Sterne, complete genome.

SEQ ID NO: 16

MIIGALACLIASMSWGAMFPVADHALEYIDPFYFSLIRYGAVAIMLIILLLMKEGKQ
AFRLEGRGKLLVFFGTMAFTVYNVLIFLGQMLMGKSGVMVASIMEALMPMISICIL
WGYKHVKPKKYMITSMLIAFIGAVFVITKGDMSFFLTLKDNMFSLACIFVGVVGWV
IYTMGGQTCSDWSTLRYSTLTCVFGTTVTGIITIIITAFGYVSVPNMGTISIVKYDLLF
MMTLPGIVALLAWNYGVKILSSINGILFINFVPITTLVIMMMQGYQITMFDIVGTLLV
IAALIRNNVCQRKEENINKRILEKEQLRQAV
BAS0205
Accession No. NC_005945, REGION: 205187 . . . 206140
Bacillus anthracis str. Sterne, complete genome.
Bases 1 to 867
ORIGIN

SEQ ID NO: 17

| | |
|---|---|
| 1 | atggaattaa gagacttgca aatcttccag agcgttgccg accaaggtag tgtaagtagc |
| 61 | gcagcaaagg aattaaatta cgtacaatca aatgtaactg cacgtattaa acaactagaa |
| 121 | aacgagctaa aaacaccgct cttttatcgt cataagcgag gcatgacttt aacagctgaa |
| 181 | ggaagaaaaa tgctcgttta tgttaataaa attttgcaag atgttgacga gctaaaacaa |
| 241 | gtatttttag atagcgaaac accctctggc atattaaaaa tcggtactgt cgaaacagta |
| 301 | agtacattac ctaccatttt atcttcttac tataaaagct atccgaacgt cgatttgtca |
| 361 | ttacaagcag gtttaacaga agaattaatt agagaagtac tcgatcatca attagatggc |
| 421 | gcttttatat caggaccaat aaaacatcca cttattgaac aatacgatgt tagtacagaa |
| 481 | aaattaatgc ttgtaacaca aaataaaact tttcatattg aagaatttac aacaacgcct |
| 541 | ctactcgttt ttaatcaagg atgtggatac cgttctaaac tagaacgatg gctgaaagat |
| 601 | gaaggtttgc ttccaaaaag aattatggaa ttcaatatat tagagacact attaaacagt |
| 661 | gttgcactcg gccttggaat tacactcgta ccacagtctg ctgtccatca tctttctaaa |
| 721 | gcaggtaaag ttcattgcca tgcaattcct gagaaatatg gtagtatttc aacggttttc |
| 781 | atacgccgca aagatagcta tatgacgaat tcaatgcgta gctttttaaa aacaatcgaa |
| 841 | gagcaccacc acattaatat gctttaa |

BAS0205
Accession No. NC_005945
Bacillus anthracis str. Sterne, complete genome.

SEQ ID NO: 18

MELRDLQIFQSVADQGSVSSAAKELNYVQSNVTARIKQLENELKTPLFYRHKRGMT
LTAEGRKMLVYVNKILQDVDELKQVFLDSETPSGILKIGTVETVSTLPTILSSYYKSY
PNVDLSLQAGLTEELIREVLDHQLDGAFISGPIKHPLIEQYDVSTEKLMLVTQNKTFH
IEEFTTTPLLVFNQGCGYRSKLERWLKDEGLLPKRIMEFNILETLLNSVALGLGITLV
PQSAVHHLSKAGKVHCHAIPEKYGSISTVFIRRKDSYMTNSMRSFLKTIEEHHHINML
BAS1105
Accession No. NC_005945, REGION: 1158156 . . . 1159091
Bacillus anthracis str. Sterne, complete genome.
Bases 1 to 936
ORIGIN

SEQ ID NO: 19

| | |
|---|---|
| 1 | atgactaaac aacgtgagaa attaattgaa gtaaaaaatg taaagcaaca cttcgacgtg |
| 61 | agtggtggtg ttgtcaaagc ggttaacgat atttcatttg atatttaccg cggagaaaca |
| 121 | tttggtcttg taggagaatc gggttgtggt aaatcaacaa ctggaagaac gattattcgt |
| 181 | ttatatgatg caactgctgg tgaagtgttg ttcgacggtg aaaatgtaca tggtaaaaaa |
| 241 | tcacgcgcag aactgaagaa attcaatcgt aaaatgcaaa tgattttcca agatccatat |
| 301 | gcatcattaa accctcgtat gacagtaggg gatattattg cagaaggtat cgatattcac |
| 361 | ggactagcaa aaaacaaaaa agagcgtatg gaccgtgttc atgaattatt aaacacagtt |
| 421 | ggcttaaata aagagcacgc aaaccgtttc ccgcatgaat tctcaggtgg acaacgtcag |
| 481 | cgtatcggta tcgctcgtgc acttgctgta gaacctgaat ttatcattgc cgatgagcca |
| 541 | atctcagcac ttgacgtatc aattcaggcg caagttgtaa acttactgaa aaagttacaa |
| 601 | aaagaaaaag gtttaacata cttattcatt gcccatgatt tatcaatggt aaaatacatt |
| 661 | agtgaccgca ttggtgtaat gtaccgtggt caaatcgttg aattaacaac aagtgatgag |
| 721 | ttatatgcga atccaattca tccatatact aaatcactat tatcagcgat tccacttcca |
| 781 | gatccagatt atgagcgtaa tcgtaaacgt atcgtatacg atccatctca gcataattat |
| 841 | ggtagtgaag aaccgacaat gcgtgaaatt cgcccaggac attttgtact atgttctgaa |
| 901 | gcggagtata agaaatataa agagatttat caataa |

BAS1105
Accession No. NC_005945
Bacillus anthracis str. Sterne, complete genome.

SEQ ID NO: 20

MTKQREKLIEVKNVKQHFDVSGGVVKAVNDISFDIYRGETFGLVGESGCGKSTTGR
TIIRLYDATAGEVLFDGENVHGKKSRAELKKFNRKMQMIFQDPYASLNPRMTVGDI
IAEGIDIHGLAKNKKERMDRVHELLNTVGLNKEHANRFPHEFSGGQRQRIGIARAL
AVEPEFIIADEPISALDVSIQAQVVNLLKKLQKEKGLTYLFIAHDLSMVKYISDRIGV
MYRGQIVELTTSDELYANPIHPYTKSLLSAIPLPDPDYERNRKRIVYDPSQHNYGSEE
PTMREIRPGHFVLCSEAEYKKYKEIYQ
BAS3376
Accession No. NC_005945, REGION: complement(3348101 . . . 3348820)
Bacillus anthracis str. Sterne, complete genome.
Bases 1 to 720
ORIGIN

SEQ ID NO: 21

| | |
|---|---|
| 1 | atgaaacgaa acacatacat cgatttctta gcgtattacg gaatagggag tgctcaccct |
| 61 | ggtggtttta cgttaacaaa acaattgtta gcacaactgc cttttagata tggagctaac |
| 121 | gtccttgaga taggctgcgg tacggggaaa acagcagcgt atatgacaaa agactgtggt |
| 181 | tataaagtaa cggcggttga aaagaatgag attatgattc aaaaggcgaa agataggtgg |
| 241 | tcgtctgaag gaatagatat tcaattaatt gaaggaaagg cagagcaatt accttgtttg |
| 301 | catgactcat ttgaattcgt actcggagaa tcgatacttg cttttacaga gaaagaaagg |
| 361 | gttatctcgg agtgctatcg tgtattacag aaggacggaa agttagttgt aattgaaatg |
| 421 | attattaatg cccacattgg gagggaagag gaagaaaaaa tcgctcaatt atatggcatg |
| 481 | aaagaactat taactgagaa tgagtgggta caattatttc agaaagcaaa ttttaaaaga |
| 541 | attacaattg ctggcggtgg cacgattgca gaaacgattt ctagctatgt agaagagcca |
| 601 | gaatggaatg tatcacaatt tattccgaat gagttatatg aggcatgggt acagcatgaa |
| 661 | aatgtacggc ttatgtacca acatatttta gggcatcgta tttttatatg tgaaaaataa |

BAS3376
Accession No. NC_005945
Bacillus anthracis str. Sterne, complete genome.

SEQ ID NO: 22

MKRNTYIDFLAYYGIGSAHPGGFTLTKQLLAQLPFRYGANVLEIGCGTGKTAAYMT
KDCGYKVTAVEKNEIMIQKAKDRWSSEGIDIQLIEGKAEQLPCLHDSFEFVLGESIL
AFTEKERVISECYRVLQKDGKLVVIEMIINAHIGREEEEKIAQLYGMKELLTENEWV
QLFQKANFKRITIAGGGTIAETISSYVEEPEWNVSQFIPNELYEAWVQHENVRLMYQ
HILGHRIFICEK
BAS3377
Accession No. NC_005945, REGION: complement(3348933 . . . 3350639)
Bacillus anthracis str. Sterne, complete genome.
Bases 1 to 1707
ORIGIN

SEQ ID NO: 23

| | |
|---|---|
| 1 | atgaagaaaa agatgaaaaa attcacggca gttgtagcac cagttttggc aatgagtatg |
| 61 | gctttaacag catgttctgg atcttctggt ggggagaaga aatcgactac aacgtctaat |
| 121 | aatggtgggg aagagaagaa gtctgatatt aaatatgcgg cgaagcaagt gttaaatcgt |
| 181 | acagaaacga atgaaattcc gacgatggat acttccaaaa atacagatac acttggctca |
| 241 | caaattttag ggaatacaat ggaagggtta tatcgccttg ataaaaacaa taagccaatc |
| 301 | ccagctgtag cagaatctag cacaaaaagc gaggatggta aaaaatatac atttaaacta |
| 361 | cgtaaagatg caaaatggtc aaacggtgat ccagtaacag cgaaagattt cgtatttgca |
| 421 | tggcaacgtc tagtagatcc aaaaaaagct gctgagtatg catttatcgc ttactatatt |
| 481 | aaaaatgcgg aaacaattaa tcaaggaaaa ggagaagttt ctacattagg tgtaaaagcg |
| 541 | gtagatgatt atacgcttga agtggaacta gaaagaccag taccatattt cttgaactta |
| 601 | atggcatttg cgtcttacta tccattaaat gaaaagtttg tgaaagaaaa agggaataaa |
| 661 | ttcggtttag agtctgatac aacactttat aatggaccat tcgtgcttac tgattggaag |
| 721 | catgagcaag gttggaaatt aaagaaaaat gagcagtatt gggacaaaaa gactgtcaaa |
| 781 | ctagaagaaa tcaattatag tgtagtaaaa gaaccagcta ctagagtaaa tttatatgac |
| 841 | acaggtgcga ttgatttcac acttttatca ggtgaatttg ttgataagta tagaaataat |
| 901 | aaagaagaat ttggtgcata ttcggaaaca agtacgtttt atttacgtct aaaccaaaaa |
| 961 | cgtggtgggc aagatacacc gttaaagagc aaaaaactac gtgaagcaat tgcattatca |
| 1021 | attgataaaa aagctttaac gaatgttatt ttaaatgatg gttcaaaacc agtggattat |
| 1081 | ttagtaccaa aaggtttagc gagtggacca gacggtaaag atttcgcaga aacgttcaaa |
| 1141 | aatggtttaa aacaagactc caaaaaggca gcggcagcct gggaagaagc gaaaaaagaa |
| 1201 | cttggaaaag atcaagtcac aattgaactg ttaaactatg atactggtaa tgcgaaaaaa |
| 1261 | gttggggagt atgtaaaaga ccaagttgaa aagaatttaa aaggtgtaac agtaaatatt |
| 1321 | aaactgcagc catttaagca aaaactaaaa ttagaatcag accaagatta tgatttctca |
| 1381 | tatggcggct ggaatccaga ttatgcggat ccaatgacat accttgatat gtttgaaaca |
| 1441 | aaaaattcgc agaaccagat gagctactca aattcaaaat atgatgacat tattactaaa |
| 1501 | agtaagacag aatggatggc tgatgcgaaa aaacgttgga cagagttagg gaaagcagaa |
| 1561 | aaattgttac ttgaagaaga tgtagcgctt gtgcctttat atcaaactgc tagatcatat |
| 1621 | gttatgaaac caaatataaa gggaattgtg aaacataata ttagtccgga atatagcttt |
| 1681 | aaatgggcgt atgtagaaga gaaataa |

BAS3377
Accession No. NC_005945
Bacillus anthracis str. Sterne, complete genome.

SEQ ID NO: 24

MKKKMKKFTAVVAPVLAMSMALTACSGSSGGEKKSTTTSNNGGEEKKSDIKYAA
KQVLNRTETNEIPTMDTSKNTDTLGSQILGNTMEGLYRLDKNNKPIPAVAESSTKSE
DGKKYTFKLRKDAKWSNGDPVTAKDFVFAWQRLVDPKKAAEYAFIAYYIKNAETI
NQGKGEVSTLGVKAVDDYTLEVELERPVPYFLNLMAFASYYPLNEKFVKEKGNKF
GLESDTTLYNGPFVLTDWKHEQGWKLKKNEQYWDKKTVKLEEINYSVVKEPATR
VNLYDTGAIDFTLLSGEFVDKYRNNKEEFGAYSETSTFYLRLNQKRGGQDTPLKSK
KLREAIALSIDKKALTNVILNDGSKPVDYLVPKGLASGPDGKDFAETFKNGLKQDS
KKAAAAWEEAKKELGKDQVTIELLNYDTGNAKKVGEYVKDQVEKNLKGVTVNIK
LQPFKQKLKLESDQDYDFSYGGWNPDYADPMTYLDMFETKNSQNQMSYSNSKYD
DIITKSKTEWMADAKKRWTELGKAEKLLLEEDVALVPLYQTARSYVMKPNIKGIV
KHNISPEYSFKWAYVEEK
BAS4394
Accession No. NC_005945, REGION: 4306678 . . . 4307985
Bacillus anthracis str. Sterne, complete genome.
Bases 1 to 1308
ORIGIN

SEQ ID NO: 25

| | |
|---|---|
| 1 | atgctggaaa aatcgtcgaa tatgcagata tcgagcatat atttactagt ccaaaacacc |
| 61 | cttatacaat cggacttctt caatctcttc caagtctcga tacagaccaa gaagaattac |
| 121 | aaacgattcc tggatccgta ccgagtccat accatatgcc gagtggatgt cgcttcgctg |
| 181 | atagatacac acatgcaaaa gaactatgtc acaatactct tccagaactt caactcacgc |
| 241 | aagatggaag tgaagttcga tgctggatgt tcactgacct ttgggataaa tcatcttcag |
| 301 | aaaaattgga ggtattataa aatgtctact actacgcaaa taaataaacg agatttatta |
| 361 | caagtgcaaa atttaaaaca atacttccct ataaaaaaag gaattctagg acgctctatt |
| 421 | agctatatta aagcggttga cgatattagt tttacagttt atgaaaagga gactgttagt |
| 481 | attgttggtg aatctgggtg cggaaagtcc accactgggc gtgcaatatt gcgccttgat |
| 541 | gaagcgacaa gtggaaaaat tatatttcaa gataaagatt tactagcatt aaataactca |
| 601 | gcaatgcgaa aggttcgaaa agatttacaa gttatttttc aagatccctt cgcttcttta |
| 661 | aaccctcggc aaactgtagg aagcatttta gaagaagcta tgtccattca aaacgtatgt |
| 721 | ccaaaagggg aaagaaaagc aaaagtaatt gagttactcg ggaaagttgg tcttccacct |
| 781 | gatgcagtga agcgctatcc acatgaattt agtggtggtc aacggcaaag aattggaatc |
| 841 | gcgcgcgctt tagctgtgaa tccaaaactc atcatttgtg acgaagccgt ctccgcctta |
| 901 | gatgtttcag tgcaagcaca agttttaaat ttattaaagc agttgcaaca acaatatggt |
| 961 | ttaacgtact tattcatctc tcatgactta gctgtcgttc gtcacatatc agatcgcatc |
| 1021 | attgtaatgt accttggtac catcgtggag attgccgata aacattctct ttttaacaat |
| 1081 | ccgcaacacc cttacacaaa agcgcttctc tcagcaattc ctaccattag tgcaggaacg |
| 1141 | aaaaaagagc gtattgaact taaaggagac ctcccctctc ctttaaatcc gccaacaggc |
| 1201 | tgtcgctttc atactcgttg tccgtatgct attgaaaaat gcgctacgca acaaccaagt |
| 1261 | tttcaatcta taagtaaaga tcataaagta gcctgtcata ttatttaa |

BAS4394
Accession No. NC_005945
Bacillus anthracis str. Sterne, complete genome.

SEQ ID NO: 26

MLEKSSNMQISSIYLLVQNTLIQSDFFNLFQVSIQTKKNYKRFLDPYRVHTICRVDV
ASLIDTHMQKNYVTILFQNFNSRKMEVKFDAGCSLTFGINHLQKNWRYYKMSTTT
QINKRDLLQVQNLKQYFPIKKGILGRSISYIKAVDDISFTVYEKETVSIVGESGCGKST
TGRAILRLDEATSGKIIFQDKDLLALNNSAMRKVRKDLQVIFQDPFASLNIPRQTVGSI
LEEAMSIQNVCPKGERKAKVIELLGKVGLPPDAVKRYPHEFSGGQRQRIGIARALA
VNPKLIICDEAVSALDVSVQAQVLNLLKQLQQQYGLTYLFISHDLAVVRHISDRIIV
MYLGTIVEIADKHSLFNNPQHPYTKALLSAIPTISAGTKKERIELKGDLPSPLNPPTGC
RFHTRCPYAIEKCATQQPSFQSISKDHKVACHII
BAS4648
Accession No. NC_005945, REGION: 4543055 . . . 4543888
Bacillus anthracis str. Sterne, complete genome.
Bases 1 to 834
ORIGIN

SEQ ID NO: 27

| | |
|---|---|
| 1 | ttgctagtag aactaagggg aattcaaaag aaatatggca aatcacttat actagacaac |
| 61 | attgatttat ccattccaga aggagaagca ctggctatta tcggcgggaa cgggactgga |
| 121 | aaaagtactc tactcaaaat aattgcaggt tttatttcac ctacggcagg gacaattcaa |
| 181 | agaaaagaac atatacaaat cggttatgta cctgaacatt ttcctgaagg gattcgtttt |
| 241 | acattagagg attatctata ccatctcggc cacattcacg gtttatcaac aaaatatgta |
| 301 | aaagataaaa ttccgatgct tctggaatct tttcatttac atcatgcaag acattctgtt |
| 361 | gtacgaaact tttcaaaggg catgaaacaa aaaacaggta ttatgcaagc attacttacg |
| 421 | gacgtacatt tattaatttt ggacgaaccc ctttctggac tcgatcctaa ctctcagcaa |
| 481 | gaattagaac atattttact ctcattaaaa caacaaggta tatccgtgtt atttacatgt |
| 541 | cacgaaaaac aactattaga aaacttcgct gatagaattg tgacgttagc aaatcataca |
| 601 | atcacagaaa atatctctgc acaaaaagga gcagagcggg tctatattga agcaatcgtt |
| 661 | cacgaaacat tttcagcgat agaactacaa aaacaatccg gttttataca cgttgcacac |
| 721 | aattcaaatc aaaaccttat tcaattgcac atcgaaaaag aacatacaaa tgacatactt |
| 781 | cagtttttat tacataaaaa agcatctatc acactgctac aacctaactt ttaa |

BAS4648
Accession No. NC_005945
Bacillus anthracis str. Sterne, complete genome.

SEQ ID NO: 28

MLVELRGIQKKYGKSLILDNIDLSIPEGEALAIIGGNGTGKSTLLKIIAGFISPTAGTIQ
RKEHIQIGYVPEHFPEGIRFTLEDYLYHLGHIHGLSTKYVKDKIPMLLESFHLHHARH
SVVRNFSKGMKQKTGIMQALLTDVHLLILDEPLSGLDPNSQQELEHILLSLKQQGIS
VLFTCHEKQLLENFADRIVTLANHTITENISAQKGAERVYIEAIVHETFSAIELQKQS
GFIHVAHNSNQNLIQLHIEKEHTNDILQFLLHKKASITLLQPNF
BAS4647
Accession No. NC_005945, REGION: 4542285 . . . 4542857
Bacillus anthracis str. Sterne, complete genome.
Bases 1 to 573
ORIGIN

SEQ ID NO: 29

| | |
|---|---|
| 1 | atgaaattag aacgtgtatt accgtttgct cgctcgcttc tgcaaacggc tgttaaagaa |
| 61 | ggcgattatg ctgtagatgc aactttagga aacggtcatg acacttgctt cctagctgaa |
| 121 | atcgttggag atagcggaaa agtatttgga tttgatattc aaaaagaagc aattgaaagc |
| 181 | tcgacgaccc gtttaaaaga aaaagaactt ttcgaacgta ctgttttagt tcacgatagt |
| 241 | cacgatacac tgctatccgt attaccagaa gatgcaaagg gaaaagtaac aggcgcaatc |
| 301 | ttcaacttag gttaccttcc aggcggagac aagcatatcg ttacaaaacc gaactcaaca |
| 361 | atttcagcga tcgaacaatt actagaagta atggcacctg aaggtatcat cgtccttgtc |
| 421 | atttaccacg gacacccaga aggacaagta gaacgcgacg ctgttctcaa atttgccgaa |
| 481 | gaactagacc aaaaacaagc acacgtactg cgatacggct tcattaacca gcaaaataac |
| 541 | ccgccattta ttgtggcgat tgagaaacga taa |

BAS4647
Accession No. NC_005945
Bacillus anthracis str. Sterne, complete genome.

SEQ ID NO: 30

MKLERVLPFARSLLQTAVKEGDYAVDATLGNGHDTCFLAEIVGDSGKVFGFDIQKE
AIESSTTRLKEKELFERTVLVHDSHDTLLSVLPEDAKGKVTGAIFNLGYLPGGDKHI
VTKPNSTISAIEQLLEVMAPEGIIVLVIYHGHPEGQVERDAVLKFAEELDQKQAHVL
RYGFINQQNNPPFIVAIEKR
BAS3035
Accession No. NC_005945, REGION: complement(3007882 . . . 3008574)
Bacillus anthracis str. Sterne, complete genome.
Bases 1 to 693
ORIGIN

SEQ ID NO: 31

| | |
|---|---|
| 1 | atgaatacgg atgtacaaga gtctttttta aacaatatcg cacgtaaatt aaatcgagaa |
| 61 | cgtcgctcgg gagttactcc tcccaggtgg aaaaacaatc cacttagcca cttttctaat |
| 121 | gagatagatc acaaaagttt agtagagcag tttattgcaa acttgcatac gttacataca |
| 181 | gaagtaagtc atatacaccg ttcagaaata gggagtgcac tacagtatgt tgtacataaa |
| 241 | tttaatattc aatctgcggt gtattgggac gatgatagat tacatcaact tgaaatagga |
| 301 | aagcatttaa taggaaattt cgtttctcat cgaatgtggc aaagtaaaga aggggaaaga |
| 361 | gaactacggg attatgcagc tcaagtggat atgggaatta catatgctga aatgggactc |
| 421 | gctgagacgg gaaccgttgt tttatggaat ggtggtggac gcgggcgttt agttagcgtt |
| 481 | ttgccagcag tttatgtagc aattctctca gaacatacaa tttatagacg cttaacagaa |
| 541 | ggagtaacta gaattcatga acaggtttcg aatggattac ctgcctgtat taattttatc |
| 601 | actgggccta gccggacagg tgatattgag atggaattag cttttggagt tcatggccca |
| 661 | ggcaaggtcc atgtcattct attaaaggat tag |

BAS3035
Accession No. NC_005945
Bacillus anthracis str. Sterne, complete genome.

SEQ ID NO: 32

MNTDVQESFLNNIARKLNRERRSGVTPPRWKNNPLSHFSNEIDHKSLVEQFIANLHT
LHTEVSHIHRSEIGSALQYVVHKFNIQSAVYWDDDRLHQLEIGKHLIGNFVSHRMW
QSKEGERELRDYAAQVDMGITYAEMGLAETGTVVLWNGGGRGRLVSVLPAVYVA
ILSEHTIYRRLTEGVTRIHEQVSNGLPACINFITGPSRTGDIEMELAFGVHGPGKVHVI
LLKD
BAS3034
Accession No. NC_005945, REGION: complement(3006489 . . . 3007841)
Bacillus anthracis str. Sterne, complete genome.
Bases 1 to 1353
ORIGIN

SEQ ID NO: 33

| | |
|---|---|
| 1 | atgaagaata aatttcgtta ttatgtcttt acaatgctca cgtttattac gatagtaaat |
| 61 | tatattgatc gaggggccat cgcttatgcg cagtctttca ttataaagga atatggcttt |
| 121 | gacccgaagg agtggggagc tatattaggg tattttggtt acggttacat gattggttct |
| 181 | ttattaggag gtattttttc agataaaaaa ggaccgaaat ttgtatggat tgtagcagcg |
| 241 | acggcttggt ctatttttga aattgcgact gcttttgctg gagaaatagg gattgctgtt |
| 301 | tttggagggt ctgctttaat aggatttgct attttccgcg ttttatttgg attaacagaa |
| 361 | ggtccatctt ttgcggtttc gaataagaca gcagcaaact gggcagctcc aaaagaaagg |
| 421 | gcttttctaa catcccttgg ttttgttggc gttccgttag gggcagtatt aacagcacct |
| 481 | gtagcggttc tgttgctatc tttcactagt tggaaaatca tgttttttat cctcggtaca |
| 541 | atagggattg tatgggcgat tatttggtat tttactttta cgaatatgcc tgaggatcat |
| 601 | ccacgagtga caaaagaaga actagctgaa atacgaagta cggaaggtgt gcttcaatca |
| 661 | gcaaaagtag agaaagaaat tccaaaagag ccatggtact ccttttttaa agttccgaca |
| 721 | ttcgttatgg ttacgatagc atatttttgc ttccaatata tcaatttttt aatattaact |
| 781 | tggacaccaa aatacttgca agatgtattc cattttcaat tatcttccct ttggtatctt |
| 841 | gggatgattc cttggctcgg agcttgcatc acattgccac taggggcgaa gctatctgat |
| 901 | cgtattttac gtaaaacagg aaaccttcgt ttagctcgaa ccgggttacc gattattgct |
| 961 | ttattactga cagcaatttg ttttagcttc attccagcga tgaataatta cgtagctgta |
| 1021 | ttagcgctta tgtcgcttgg aaatgcgttt gcttttttac caagttcatt attttgggca |
| 1081 | attattgtcg atactgctcc tgcttactca ggaacatata gtggaattat gcatttcatc |
| 1141 | gctaatatcg cgacaatctt agccccgact ttaactggat acttagttgt aagttatggc |
| 1201 | tatccttcta tgtttatcgt agctgccatt ttggccgcta ttgcaatggg ggcaatgttg |
| 1261 | tttgtgaaac cagggcagca gacgaagacg gaaagcttat ttaactggag aggtaagaag |
| 1321 | cggttagagg aacctcgtgc taattttgaa tga |

BAS3034
Accession No. NC_005945
Bacillus anthracis str. Sterne, complete genome.

SEQ ID NO: 34

MKNKFRYYVFTMLTFITIVNYIDRGAIAYAQSFIIKEYGFDPKEWGAILGYFGYGYM
IGSLLGGIFSDKKGPKFVWIVAATAWSIFEIATAFAGEIGIAVFGGSALIGFAIFRVLF
GLTEGPSFAVSNKTAANWAAPKERAFLTSLGFVGVPLGAVLTAPVAVLLLSFTSWK
IMFFILGTIGIVWAIIWYFTFTNMPEDHPRVTKEELAEIRSTEGVLQSAKVEKEIPKEP
WYSFFKVPTFVMVTIAYFCFQYINFLILTWTPKYLQDVFHFQLSSLWYLGMIPWLG
ACITLPLGAKLSDRILRKTGNLRLARTGLPIIALLLTAICFSFIPAMNNYVAVLALMSL
GNAFAFLPSSLFWAIIVDTAPAYSGTYSGIMHFIANIATILAPTLTGYLVVSYGYPSM
FIVAAILAAIAMGAMLFVKPGQQTKTESLFNWRGKKRLEEPRANFE
BAS2639
Accession No. NC_005945, REGION: complement(2625262 . . . 2626752)
Bacillus anthracis str. Sterne, complete genome.
Bases 1 to 1491
ORIGIN

SEQ ID NO: 35

| | |
|---|---|
| 1 | atggagcaat tagtagagtg gttagtaggg caagtgtgga gtattggttt agttgttttc |
| 61 | gcgttaggag caggtgtgta ttttacaatt gcaactcgtt ttcttcaaat tcgttatttt |
| 121 | aaagagatga ttaaactatt atttgaaggg aagagctcag aaacgggaat atcatccttt |
| 181 | caagcatttt gtttagcttt atcaggcagg gttggaatag gtaatattgc aggggtcgcg |
| 241 | acagctatcg cttttggcgg gcctggagct gtattttgga tgtgggtaat ggctctttta |
| 301 | ggagcagcta gtgcctttgt cgaatcaaca ttatctcaaa tatataaaag taaagttgaa |
| 361 | aatgaatatc gcggtggtac accgtatttc attgaaaaag gcttaaacat gaaatggttt |
| 421 | gcagtcattg tagcggtcgt tgtaacactt tcatatggtg ttttattacc aggtattcaa |
| 481 | tctagtagta tcgcagttgg attcgaaaac tctaatggga ttagcaaata tataactggt |
| 541 | atcttgttag ttgtattatt agcagcaatt atttttggtg gcgtaaagag aattgctggc |
| 601 | gtttctcaaa tgctcgttcc atttatggca attggttatg taattgttac atgtatcgta |
| 661 | ttaattgcga atgtaaaaga aatcccaagt atgttcgctt taattttctc aagtgctttt |
| 721 | ggtgtgaatg aaatgtttgg tggaatcgtc ggtgcagcaa tcgcgtgggg cgtaaagtgc |
| 781 | gctgtatttt ctaacgttgc tggcgttgga gaagcgacgt atagttcggc cgcggctgaa |
| 841 | gtatctcatc cagcaaaaca agggttagtt caagcgtttt ctgtatacat tgatacaatt |
| 901 | gtcgtatgta cagcgacagc tcttatgatc ttaataacag gtatgtataa tgttatacct |
| 961 | gaagggaaaa gcgctatcgt aaagaatata gggaatgttg atgcgggtcc aatttataca |
| 1021 | caacaagcag ttgaaactgt tatgacaggg tttggtccat tattcatttc aatcgcaatt |
| 1081 | ttcttcttcg catttacaac attacttgca tactactata tcgctgaaac gacacttact |
| 1141 | tatttagacc gtgaacttaa gcatagttgg ttaaaaccag ttttgaaaat tggattttta |
| 1201 | attatggttt acatcggtag tgtagaatca gcatcgcttt tatggaatct tggagattta |
| 1261 | ggaatcggta gtatggcatg gttaaactta atcgcgattc tactattaag taaaatcgca |
| 1321 | ttaaaagtgt taaaagatta tgaaacgcag aaaaaagaag ggaaagatcc cgtgtttgat |
| 1381 | cctaaaaatg tgggaattga aggtttaaca ttttgggaag aaagaagtaa agaggttgca |
| 1441 | agaaaaaact caaaagaaca agcggtagtg gatgatagtc tgaaattgta g |

BAS2639
Accession No. NC_005945
Bacillus anthracis str. Sterne, complete genome.

SEQ ID NO: 36

MEQLVEWLVGQVWSIGLVVFALGAGVYFTIATRFLQIRYFKEMIKLLFEGKSSETGI
SSFQAFCLALSGRVGIGNIAGVATAIAFGGPGAVFWMWVMALLGAASAFVESTLSQ
IYKSKVENEYRGGTPYFIEKGLNMKWFAVIVAVVVTLSYGVLLPGIQSSSIAVGFEN
SNGISKYITGILLVVLLAAIIFGGVKRIAGVSQMLVPFMAIGYVIVTCIVLIANVKEIPS
MFALIFSSAFGVNEMFGGIVGAAIAWGVKCAVFSNVAGVGEATYSSAAAEVSHPA
KQGLVQAFSVYIDTIVVCTATALMILITGMYNVIPEGKSAIVKNIGNVDAGPIYTQQ
AVETVMTGFGPLFISIAIFFFAFTTLLAYYYIAETTLTYLDRELKHSWLKPVLKIGFLI
MVYIGSVESASLLWNLGDLGIGSMAWLNLIAILLLSKIALKVLKDYETQKKEGKDP
VFDPKNVGIEGLTFWEERSKEVARKNSKEQAVVDDSLKL
BAS1932
Accession No. NC_005945, REGION: 1946836 . . . 1948011
Bacillus anthracis str. Sterne, complete genome.
Bases 1 to 1176
ORIGIN

SEQ ID NO: 37

| | |
|---|---|
| 1 | atgagtttga aatatggaag agatacaatt gttgaagttg acttaaatgc agtaaaacat |
| 61 | aatgtaaaag aatttaaaaa acgtgtgaat gatgaaaata ttgcaatgat ggctgctgta |
| 121 | aaagcgaatg ggtatggtca tggggcagtt gaagttgcaa aagctgctat tgaagcagga |
| 181 | ataaatcagc ttgcaattgc atttgtagat gaagcgatag agttaagaga agcaggaatt |
| 241 | aacgtgccga ttttaatttt aggctataca tcagtagcgg ctgcggaaga agcaattcaa |
| 301 | tatgacgtta tgatgaccgt ttatagaagt gaagatttac aaggtataaa tgaaatcgca |
| 361 | aaccgtcttc aaaagaaagc gcaaattcag gtgaaaattg atacaggaat gagtcgcatt |
| 421 | ggtttacagg aagaagaggt taaaccattt ttagaggaat taaaacgtat ggagtatgta |
| 481 | gaggtagtgg gaatgtttac acattactct acggcagatg aaatcgataa atcatatacg |
| 541 | aatatgcaaa caagtttatt tgagaaagct gtcaatacag caaaagaatt aggaattcat |
| 601 | attccatata ttcatagttc aaatagtgca ggttcaatgg aacctagcaa tacatttcaa |
| 661 | aatatggttc gtgtaggtat cggaatttat ggaatgtatc cttcaaaaga ggtaaatcat |
| 721 | tcagttgttt cgttacagcc tgcgttgtcg ttaaaatcaa aagtagccca tattaagcat |
| 781 | gcgaagaaaa atcgcggtgt aagttatggg aatacgtatg taacgactgg tgaagaatgg |
| 841 | attgccaccg taccgattgg ttatgctgat ggttataatc gtcagttgtc taataaaggg |
| 901 | catgcattaa taaatggagt tcgagtacct gttattggcc gtgtttgtat ggatcagctc |
| 961 | atgttagacg tttcaaaagc aatgccagta caagtgggag acgaagtagt attctacggt |
| 1021 | aaacaaggcg aagaaaacat cgcagtagaa gaaatagcgg atatgttagg tacaattaac |
| 1081 | tatgaagtta catgtatgtt agatagaaga attccacgtg tgtataaaga aaataatgaa |
| 1141 | acaactgctg ttgtaaatat actaagaaaa aactga |

BAS1932
Accession No. NC_005945
Bacillus anthracis str. Sterne, complete genome.

SEQ ID NO: 38

MSLKYGRDTIVEVDLNAVKHNVKEFKKRVNDENIAMMAAVKANGYGHGAVEVA
KAAIEAGINQLAIAFVDEAIELREAGINVPILILGYTSVAAAEEAIQYDVMMTVYRSE
DLQGINEIANRLQKKAQIQVKIDTGMSRIGLQEEEVKPFLEELKRMEYVEVVGMFT
HYSTADEIDKSYTNMQTSLFEKAVNTAKELGIHIPYIHSSNSAGSMEPSNTFQNMVR
VGIGIYGMYPSKEVNHSVVSLQPALSLKSKVAHIKHAKKNRGVSYGNTYVTTGEE
WIATVPIGYADGYNRQLSNKGHALINGVRVPVIGRVCMDQLMLDVSKAMPVQVG
DEVVFYGKQGEENIAVEEIADMLGTINYEVTCMLDPRIPRRVYKENNETTAVVNILR
KN
BAS5205
Accession No. NC_005945, REGION: 5089114 . . . 5090967
Bacillus anthracis str. Sterne, complete genome.
Bases 1 to 1854
ORIGIN

SEQ ID NO: 39

| | |
|---|---|
| 1 | atgttcaaag gaggcaaaat gaaaaaactt ttcaatatat gtttaattgt atttgtacta |
| 61 | ttttcacagt ttattagttt cccgtacaat caggcaaaag ctgagacttt aaaggaaact |
| 121 | tcattatttg acactgttga gatgaaagat gcaacggatc agattattga cgaagcaaaa |
| 181 | aatcctaaca acttaataaa gataggatca accattcaag tagaatatgc ttggtcaata |
| 241 | aaggatcaac aagttgtaca tgcaaacgat acagcagtac ttcaaatacc acttgcatta |
| 301 | aaagtatcta aagatttaca aggagattta gtaacagatc aaaaaaatat tggtcaatat |
| 361 | tttataacag ctaaagataa taaattaaaa ctaatattta atgatcaagt agaaaattcg |
| 421 | aaagacgcta aaggaaaaat taaaattgat actgtgttta acccaacttt aaagactgaa |
| 481 | gaaaaatcag ttcaaatcgc ttttccttta ggaacactgg ttcagcctat aacagttcct |
| 541 | attcaagtag aagattctaa agaagatgga accaaacagg atactaataa acaagtgcaa |
| 601 | gatcaagtag ctaaacctac tactgataat ccggaacaaa atccagcaac taaacctgct |
| 661 | actgacaatc cggaacaaaa ttcagcaact aaacctgcta ctgacaatcc ggaacaaaat |
| 721 | ccagcaacta aacctgctac tgacaatccg gaacaaaatc cagcaactaa acctgctact |
| 781 | gataatccgg aacaaaatcc agcaactaaa cctgctgctg ataacccaga acaaaatcta |
| 841 | gcaagcgatc ctgctgagat tacaaattca ggtccaaagc aaataacaac aaacatttta |
| 901 | acgggtgtaa agttgacgga caaagacgga aaaccattta cagaggataa ccgtccaagt |
| 961 | acagattccc ctgccaatat tgagtttaca tgggaacttt taaaatcaat gaatgtgaaa |
| 1021 | agcggagatt actatatttt tgatcttcct aaacatttta agatttacaa tacaattaac |
| 1081 | agccctttat acgatagtga aaacaatcca attggtaatt ttactgttac aaaagatgga |
| 1141 | aaagtcacaa tgacattcaa cgattatgtt gaagaacatc cagatgttgt tggtaaccta |
| 1201 | caattaaaga cagaatttaa taaagctgaa attaagggta caacaacaca ggaaattcct |
| 1261 | ttcccaatta aagataaaga tgtttctatt acagttgact ttaaacctaa tgtacaaacg |
| 1321 | gctacaaata aaaaagggtt acctgataga ccaattaata caaatgagat taattggaca |
| 1381 | gtagagatga acaaaacgaa agacaccctt aaaaacgctg tttttaaaga taacatccca |
| 1441 | caaggtacaa gtttaaataa ggattctatt aaagtttatt atttagaagt tgatgttaac |
| 1501 | gggaatgcaa cacgtggtca agaagctgat ccagcagatt acaaaattat ttcatcagat |
| 1561 | ggttcaaaat tggagattgc ttttaaagat tctattaaaa aagcatatca aatcgaatat |
| 1621 | gtcacaaaaa tcactgatga aaacgtaaaa agcttccaaa ataacgttac gataacaagt |
| 1681 | gataatcaag ggcaacaaaa agcaagctct actgtaacag tctctcgtgg tacacattta |
| 1741 | aacaaaacaa gtaaatatga tccaaagacc caaacaattg aatggacgat tacttacaac |
| 1801 | ggtgatcaaa gaaatatcaa aaaaaacaga tgcactttta aaagatattt ttga |

BAS5205
Accession No. NC_005945
Bacillus anthracis str. Sterne, complete genome.

SEQ ID NO: 40

MFKGGKMKKLFNICLIVFVLFSQFISFPYNQAKAETLKETSLFDTVEMKDATDQIIDE
AKNPNNLIKIGSTIQVEYAWSIKDQQVVHANDTAVLQIPLALKVSKDLQGDLVTDQ
KNIGQYFITAKDNKLKLIFNDQVENSKDAKGKIKIDTVFNPTLKTEEKSVQIAFPLGT
LVQPITVPIQVEDSKEDGTKQDTNKQVQDQVAKPTTDNPEQNPATKPATDNPEQNS
ATKPATDNPEQNPATKPATDNPEQNPATKPATDNPEQNPATKPAADNPEQNLASDP
AEITNSGPKQITTNILTGVKLTDKDGKPFTEDNRPSTDSPANIEFTWELLKSMNVKSG
DYYIFDLPKHFKIYNTINSPLYDSENNPIGNFTVTKDGKVTMTFNDYVEEHPDVVGN
LQLKTEFNKAEIKGTTTQEIPFPIKDKDVSITVDFKPNVQTATNKKGLPDRPINTNEIN
WTVEMNKTKDTLKNAVFKDNIPQGTSLNKDSIKVYYLEVDVNGNATRGQEADPA
DYKIISSDGSKLEIAFKDSIKKAYQIEYVTKITDENVKSFQNNVTITSDNQGQQKASS
TVTVSRGTHLNKTSKYDPKTQTIEWTITYNGDQRNIKKNRCTFKRYF
BAS5183
Accession No. NC_005945, REGION: complement(5063148 . . . 5064437)
Bacillus anthracis str. Sterne, complete genome.
Bases 1 to 1290
ORIGIN

SEQ ID NO: 41

| | |
|---|---|
| 1 | atggaaaaat tgctaattga aggcggaaga gctttaaatg gaacaattcg cgtgagtggt |
| 61 | gcaaagaaca gcgccgttgc attaattcca gcgacaattt tagcagatac tccagtaact |
| 121 | attggtggtg tccctaatat ttcggacgtg aaaatgttag gagacttact agaggaaatt |
| 181 | ggaggaagag taacgtatgg acaggaggaa gagatggtag tcgatccttc taacatggtt |
| 241 | gcaatgcctt taccaaacgg aaaagtgaaa aaattgcgtg cttcttatta tttaatgggt |
| 301 | gcgatgcttg gccgttttaa aaaagctgtt attgggcttc caggtggatg tcacttagga |
| 361 | ccgaggccaa ttgatcagca tattaaaggg tttgaagcgt taggtgcaca tgttacgaat |
| 421 | gaacaaggtg ctatctattt aagagcagat gaactacgcg gggctcgtat ttatttagat |
| 481 | gttgttagtg taggagctac gattaatatt atgctagcag ctgtacgagc gaaaggtaga |
| 541 | actgttattg aaaacgcagc gaaagaacca gagattattg atgtagctac actgttaact |
| 601 | agcatgggag cacgtattaa aggtgctggt acagatgtaa tccgaattga tggtgtggat |
| 661 | tcattgcacg gttgtcatca tacgatcatt ccagatcgta ttgaagcggg tacgtatatg |
| 721 | attttaggtg ctgcatcagg aggagaagta acagttgata atgttattcc tcagcactta |
| 781 | gagtcagtta cggcgaagct ccgagaagct ggtgtccagg ttgaaacgaa tgatgaccag |
| 841 | attacagtga acggtgatag aagattaaaa gtagttgata taaaaacgct tgtatatcca |
| 901 | ggtttcccaa cagacttaca acagccgttt acaacacttt taacaaaggc gcatggaacg |
| 961 | ggtgttgtaa cggatacgat ttatggtgca cgttttaaac atattgatga attacgtcgt |
| 1021 | atgaatgcac aaattaaagt agaaggtcga tcagctatcg taactggtcc tgttttattg |
| 1081 | caaggtgcaa aagtgaaagc gagtgatttg cgagctggag cagcacttgt tatcgcagga |
| 1141 | ttaatggcgg atggaattac agaagtaacc ggacttgagc atattgatcg aggttatgaa |
| 1201 | aatatagtag acaagcttaa agggcttggt gcaaacattt ggcgagaaca aatgacaaag |
| 1261 | caagaaattg aagaaatgaa gaacgcataa |

BAS5183
Accession No. NC_005945
Bacillus anthracis str. Sterne, complete genome.

SEQ ID NO: 42

MEKLLIEGGRALNGTIRVSGAKNSAVALIPATILADTPVTIGGVPNISDVKMLGDLLE
EIGGRVTYGQEEEMVVDPSNMVAMPLPNGKVKKLRASYYLMGAMLGRFKKAVIG
LPGGCHLGPRPIDQHIKGFEALGAHVTNEQGAIYLRADELRGARIYLDVVSVGATIN
IMLAAVRAKGRTVIENAAKEPEIIDVATLLTSMGARIKGAGTDVIRIDGVDSLHGCH
HTIIPDRIEAGTYMILGAASGGEVTVDNVIPQHLESVTAKLREAGVQVETNDDQITV
NGDRRLKVVDIKTLVYPGFPTDLQQPFTTLLTKAHGTGVVTDTIYGARFKHIDELRR
MNAQIKVEGRSAIVTGPVLLQGAKVKASDLRAGAALVIAGLMADGITEVTGLEHID
RGYENIVDKLKGLGANIWREQMTKQEIEEMKNA
BAS5217
Accession No. NC_005945, REGION: 5104992 . . . 5106029
Bacillus anthracis str. Sterne, complete genome.
Bases 1 to 1038
ORIGIN

SEQ ID NO: 43

| | |
|---|---|
| 1 | atggctatct tgcaaggttt agcactatta cttgttgtac tttgtctatt tacacttttt |
| 61 | agttaccgtg ctccttacgg catgaaagca atgggtgctt tagctaatgc agcaatcgca |
| 121 | agttttctta ttgaagcatt tcaccgttat atcggtggag aaatgtttca taataaattt |
| 181 | ttacaatcag taggagaagc ttctggtagt atgagcggtg tggcagcggc aattttagtc |
| 241 | gcactagcaa tcggtgtttc acccgtatat gctgttttaa ttggtatcgc tactagtgga |
| 301 | ttcggtattt taccaggatt tttcgctgga tacgtttgcg ctttcgtcgt gaaatttctt |
| 361 | gaaaagaaat taccagctgg tgtagagttt ttagcaattt tatttattgc tgcaccaatc |
| 421 | tcacgcggaa tggcaatgct tatggatccg ctcgtaaacg caacgctcgg taaaatcggt |
| 481 | tctatgattt cagttgcaac tacagaaagt cctatcatta tgggtattat gcttggtgga |
| 541 | ttaatcacag ttatttctac ctctccactg agttctatgg cactaactgc aatgctcgca |
| 601 | ttaacaggtt taccaatggc aattggtagt cttgccgtag cagcctcagc tccaatgaac |
| 661 | tttattttct ttaagcgact aaaaatttgc tcaaaaaaag acacaatcgc tgtagcaatc |
| 721 | gagcctttaa cacaagccga tgttgtttca gcaaatccaa ttccaattta tgcaacaaac |
| 781 | ttcgttggcg gtgcacttgc tggtattatt acatctctgt tccagctcgt taataacgca |
| 841 | ccaggaacag catcaccaat cccaggactt cttgtcttat tcgggtttaa tgacgttgta |
| 901 | aaagtaacga ttgccgctgt attatgtgga atcgttacca ctattgttgg gtacatcgga |
| 961 | tcaatcttgt tccgtaaata cccaattcgt tctgctgatg aaattcgcgg catttcttcg |
| 1021 | gaagagaagg ttgcataa |

BAS5217
Accession No. NC_005945
Bacillus anthracis str. Sterne, complete genome.

SEQ ID NO: 44

MAILQGLALLLVVLCLFTLFSYRAPYGMKAMGALANAAIASFLIEAFHRYIGGEMF
HNKLFLQSVGEASGSMSGVAAAILVALAIGVSPVYAVLIGIATSGFGILPGFFAGYVC
AFVVKFLEKKLPAGVEFLAILFIAAPISRGMAMLMDPLVNATLGKIGSMISVATTESP
IIMGIMLGGLITVISTSPLSSMALTAMLALTGLPMAIGSLAVAASAPMNFIFFKRLKIC
SKKDTIAVAIEPLTQADVVSANPIPIYATNFVGGALAGIITSLFQLVNNAPGTASPIPG
LLVLFGFNDVVKVTIAAVLCGIVTTIVGYIGSILFRKYPIRSADEIRGISSEEKVA
BAS1477
Accession No. NC_005945, REGION: complement(1499846 . . . 1501009)
Bacillus anthracis str. Sterne, complete genome.
Bases 1 to 1164
ORIGIN

SEQ ID NO: 45

| | |
|---|---|
| 1 | atgacacaaa atatttcaca aggcgagagg atacactcca tcgatattat tagaggaata |
| 61 | gctgtactag gtatttttct tgttaactgg cccattatcg ctgggattga ctcacgtgat |
| 121 | ctttcaggag tttacgaggg gctagatagc tatatccgtc tattttacga tatgttcatt |
| 181 | caaacaaagt tttatactat cttttcattt ttattcggcc taggctttta catctttatg |
| 241 | actcgtgctg aagcaaaaac agatcgacca aaaactttat ttgttcgtcg tttacttatt |
| 301 | ttattattat ttggtttctt acattacgtt cttttatggg acggagacat tttacatagt |
| 361 | tatgcaatcg ctggattttt cttattttta ttttataaga gaaaacctcg tactatttta |
| 421 | atatgggcaa tcgttttatt aagtattttt caatttctta tgctaatcgc tactattggt |
| 481 | attgccttca tgccaaagga ggaacttggg ttatccttac caatcatgcc acttgaagac |
| 541 | tgggtgtcac aaatacaaaa tcgtttccat gctttttatg ctaatggaat tggactaaat |
| 601 | gtatcaatgc tcccagaaac agttgggcta tttttactcg gtttatatgc cggtaaaaaa |
| 661 | gacattttcc gccgcacgaa agagttagat ccaaagctaa aaaaatggca aatcattatg |
| 721 | tttgttttaa cattaccgtt ttggttcttt atggttcgtt atttcttatc aacatcgtca |
| 781 | tatgaaccac tttatatgca agggcttgca atgtttagcg gaaaaacatt attcatcttc |
| 841 | tatattttca ctcttatgcg tttattacaa aaagaaaaat ggcaaacatt attacgtccc |
| 901 | ttccagtacg ttggtcgaat ggcattaaca aactacattt cacatacaat tgttacgtta |
| 961 | cttgtatttg gtctattgct taaaagttat tatccagctc cattatgggt aggaccacta |
| 1021 | ttttgcgtcg gtttctacac gttacaaatc tttattagcc gctggtggct gtcacgttat |
| 1081 | caatacgggc cacttgagta catttggcat cttggtacgt acgggaaaat gatgccactt |
| 1141 | aaaaagaaaa gcaaggtctc ataa |

BAS1477
Accession No. NC_005945
Bacillus anthracis str. Sterne, complete genome.

SEQ ID NO: 46

MTQNISQGERIHSIDIIRGIAVLGIFLVNWPIIAGIDSRDLSGVYEGLDSYIRLFYDMFI
QTKEYTIFSFLFGLGFYIFMTRAEAKTDRPKTLFVRRLLILLLFGFLHYVLLWDGDIL
HSYAIAGFFLFLFYKRKPRTILIWAIVLLSIFQFLMLIATIGIAFMPKEELGLSLPIMPLE
DWVSQIQNRFHAFYANGIGLNVSMLPETVGLFLLGLYAGKKDIFRRTKELDPKLKK
WQIIMFVLTLPFWFFMVRYFLSTSSYEPLYMQGLAMFSGKTLFIFYIFTLMRLLQKE
KWQTLLRPFQYVGRMALTNYISHTIVTLLVFGLLLKSYYPAPLWVGPLFCVGFYTL
QIFISRWWLSRYQYGPLEYIWHLGTYGKMMPLKKKSKVS
BAS1135
Accession No. NC_005945, REGION: 1187110 . . . 1187847
Bacillus anthracis str. Sterne, complete genome.
Bases 1 to 738
ORIGIN

SEQ ID NO: 47

| | |
|---|---|
| 1 | gtgaaaggga ttattttagc aggtggtact ggatcgagat tatatccaat aacgaaagta |
| 61 | acgaataaac atttacttcc tgttggtcgg tatccgatga tttatcatgc ggtatataag |
| 121 | ttaaaacaat gtgatattac agatattatg attattacag gtaaagagca tatgggggat |
| 181 | gttgttagct ttttagggag cggtcaagag tttggcgtgt cctttacgta tcgtgtgcaa |
| 241 | gataaagctg gcggaattgc acaagcatta gggctttgtg aagattttgt tgggaatgat |
| 301 | cgcatggtag ttatattagg tgataatatt ttttcagatg atattcgtcc gtatgttgaa |
| 361 | gagtttacaa atcaaaaaga aggtgcgaaa gtactgctgc aatctgtaga tgatccggag |
| 421 | agatttggcg tagcaaatat tcaaaaccgc aaaataattg aaattgaaga aaagccgaaa |
| 481 | gagccgaaaa gttcctatgc agttacagga atttacttgt atgattcgaa agtcttttct |
| 541 | tatataaaag aattaaaacc ttccgcaagg ggagaacttg aaattacaga tatcaataat |
| 601 | tggtatttaa agcgaggggt acttacttat aatgaaatga gcggttggtg gactgatgcg |
| 661 | ggaactcatg tttctcttca aagagcgaat gcgttagcac gggatataaa ctttggtaaa |
| 721 | cagtttaacg gagaatag |

BAS1135
Accession No. NC_005945
Bacillus anthracis str. Sterne, complete genome.

SEQ ID NO: 48

MKGIILAGGTGSRLYPITKVTNKHLLPVGRYPMIYHAVYKLKQCDITDIMIITGKEH
MGDVVSFLGSGQEFGVSFTYRVQDKAGGIAQALGLCEDFVGNDRMVVILGDNIFS
DDIRPYVEEFTNQKEGAKVLLQSVDDPERFGVANIQNRKIIEIEEKPKEPKSSYAVTG
IYLYDSKVFSYIKELKPSARGELEITDINNWYLKRGVLTYNEMSGWWTDAGTHVSL
QRANALARDINFGKQFNGE
BAS5285
Accession No. NC_005945, REGION: complement(5168832 . . . 5170271)
Bacillus anthracis str. Sterne, complete genome.
Bases 1 to 1440
ORIGIN

SEQ ID NO: 49

| | |
|---|---|
| 1 | ttggaacgtt tttctggatt attttcaact ttttctacac gtgccatata cggcatttat |
| 61 | agctttttcg ttgcaatatt aatttttgat tatttttcag atactcaaaa gtataattat |
| 121 | gtaatgatat tagttggagt tttattatta tgtacaattg gaaactttat acttagcagt |
| 181 | ggatcgctat atcttgaaag agtaaatgaa aaaatatgtt tttttgtact actaattatt |
| 241 | tgtttagcag tgaaaactgc atggattgtt acatataaga ttgatccgat tggtgattat |
| 301 | gaagcctttt tcaatactgc aaaagcatta ggtgataact ttgttattca tgatagatat |
| 361 | gtagcactat ttccacatat ttttggttat gcttcattct taagtatctt cctaaaaata |
| 421 | tttggggcaa actttatgat tccgccaatt attaatgttg ttttaactac aatttcgatg |
| 481 | ggattaatat attttatagc tagacgaatt ggtggagtga gaacagcgat aacagcaagt |
| 541 | gttttatgga ttctcttacc atcacaaacg atgtataaca tgtttgcact ttcagaaccg |
| 601 | ttgtattgta caatactgtt attagcctgg gcaattatga taattgttta tgataaaatt |
| 661 | gaaaatatga agattgcaaa ggttcttatg tattcaattc tattagctgc attacttgta |
| 721 | ttaataaata tggcaagacc aattgcagca gtgccaatta tagccttagc gatatggatg |
| 781 | tttattatag atacaaagca tatcgggaat aaaaagctac ttattaataa gttggcatac |
| 841 | gtaggggtta tcattattgg atacttagtg atgtcttcag cggcaaatca ttatgtaaca |
| 901 | ttacgtttag gtgaagagat agcaacagtg ccgggataca atattcatgt aggatttaat |
| 961 | aagggagcct caggaacatg gaatccagga gattcagcgt tactatatca ttatagtggt |
| 1021 | caaccaggat ggagtgctca ggacgttcaa aagcaaatgc ttgaagaagc gaaaaagaga |
| 1081 | atcaaaaacg atgatataga tttcgggaaa ctaatgtatg ataaatttat tatcttttta |
| 1141 | ggtaatgatg atcaagctgt taaatatgca gatccaatta tggaccataa agtacgctat |
| 1201 | actataattt ctaatgtttt ctattacttt ttactagtta cttcactgtt cggagcgtta |
| 1261 | gtagctataa aaaataaaaa taaatcttca cttttaatta tttgtttata tgtgattggg |
| 1321 | ctaacaatgg cacaaatgat agtagaagta gcaccgagat atcattattc ggctacaata |
| 1381 | cctatgatct ttttagctgc ttttggcatt aagcatattt acaataaaaa aagaatataa |

BAS5285
Accession No. NC_005945
Bacillus anthracis str. Sterne, complete genome.

SEQ ID NO: 50

MERFSGLFSTFSTRAIYGIYSFFVAILIFDYFSDTQKYNYVMILVGVLLLCTIGNFILSS
GSLYLERVNEKICFFVLLIICLAVKTAWIVTYKIDPIGDYEAFFNTAKALGDNFVIHD
RYVALFPHIFGYASFLSIFLKIFGANFMIPPIINVVLTTISMGLIYFIARRIGGVRTAITA
SVLWILLPSQTMYNMFALSEPLYCTILLLAWAIMIIVYDKIENMKIAKVLMYSILLA
ALLVLINMARPIAAVPIIALAIWMFIIDTKHIGNKKLINKLAYVGVIIIGYLVMSSAA
NHYVTLRLGEEIATVPGYNIHVGFNKGASGTWNPGDSALLYHYSGQPGWSAQDVQ
KQMLEEAKKRIKNDDIDFGKLMYDKFIIFLGNDDQAVKYADPIMDHKVRYTIISNV
FYYFLLVTSLFGALVAIKNKNKSSLLIICLYVIGLTMAQMIVEVAPRYHYSATIPMIF
LAAFGIKHIYNKKRI
BAS0638
Accession No. NC_005945, REGION: 688776 . . . 691175
Bacillus anthracis str. Sterne, complete genome.
Bases 1 to 2400
ORIGIN

SEQ ID NO: 51

| | |
|---|---|
| 1 | atgagaagaa aagcgccact taaagtgtta tcgtcattag caattgcggc aattatcgga |
| 61 | tgtacatctg taatgagtgc tccattagcg tacgcagaaa cgccagcaaa agagaaagaa |
| 121 | aatgtatcta caacaccaat tgattacaat ttaattcaag aagatcgtct agcggaagcg |
| 181 | ctgaaagaaa gaggaacaat taatccagca tcttctaaag aagagacgaa aaaggctgta |
| 241 | gagaaatata ttgaaaagaa acaaggagac caggcaaata aagaaattct tccagctgat |
| 301 | actgctaaag aggcatctga tttcgtgaaa aaagtaaaag agaaaaaaat ggaagaaaag |
| 361 | gagaaagtaa agaaacctga aaaaaatgtt agccctgagc aaaagcctga accaaataaa |
| 421 | aaacaattga atggacaagt tccaacatct aaagcaaagc aagcgccata taaggggtct |
| 481 | gttcgaacgg ataaagtatt agtattactc gttgaattta gtgattataa acataataat |
| 541 | attgatcaaa caccagggta tatgtattcg aatgacttta gtagagagca ttatcaaaag |
| 601 | atgttatttg gtaatgagcc gtacacatta tttgatggtt caaaagtaaa aacgtttaaa |
| 661 | caatattatg aagagcagtc tggcggtagt tatacgactg atggatatgt aacagaatgg |
| 721 | ttaactgttc caggaaaagc atctgactac ggtgctgatg gtagcagtgg tcatgataac |
| 781 | aaaggtccaa aaggcgcacg tgatttagtg aaagaagctt tacatgcagc tgctgagaaa |
| 841 | ggtttagatt tatctcaatt tgatcagttt gatagatatg atacaaatag tgatggaaat |
| 901 | caaaatgaac ctgatggtgt aattgatcat ttaatggtaa tccatgctgg tgttggtcaa |
| 961 | gaagctggtg gaggtaaatt aggtgatgat gccatttggt cacatcgttc aaaattagca |
| 1021 | atagatccag tagcaattga agggacaaaa tcaaaggtag attactttgg tggcaaagta |
| 1081 | gcagcacatg attacacaat tgaaccagaa gatggagcag taggtgtatt tgcgcatgaa |
| 1141 | tttggacatg atcttggctt accagatgaa tatgatacga aatatactgg aactggttca |
| 1201 | cctgtcgaag cttggtcatt aatgagtgga ggtagttgga cagggaaaat tgcaggaaca |
| 1261 | gagccaacta gtttttcacc acaaaataaa gatttcttac aaaagaatat gggtggcaac |
| 1321 | tgggcaaaaa ttttagaagt agattacgat aaaattaagc gtggtgtagg agttcctaca |
| 1381 | tatattgatc aaagtgttac gaaatcaaat cgtccaggcg ttgtacgtgt taacttacca |
| 1441 | ggcaaaagtg ttgaaacgat taaaccggag tttggaaagc atgcatatta tagtacaaga |
| 1501 | ggcgatgata tgcatacaac attagaaaca ccgttctttg atttaacaaa aggaacaaat |
| 1561 | gcaaagtttg attataaagc aaattatgag ttagaagcag agtgcgattt tgttgaagtt |
| 1621 | cacgcagtaa cagaagatgg aacgaaaaca ttaattgata gacttggaga aaaagtagtc |
| 1681 | caaggagata aagatacaac agatggaaaa tggattgata aatcatatga tttaagtcaa |
| 1741 | tttaaaggaa aaaaagtgaa actgcaattc gattatatta cagatccagc tgtaacatat |
| 1801 | aaaggtttcg cgatggatca tgtaaatgta actgttgatg gacaagtagt attttctgat |
| 1861 | gatgcagaag gacagtctaa aatgaattta aatggttttg ttgtttctga tgggacagag |
| 1921 | aaaaaagctc attattacta cttagagtgg agaaactatg caggatcaga taatggatta |
| 1981 | aaagcaggaa aaggtccagt gtataataca ggtcttgtcg tttggtatgc agatgatagc |
| 2041 | tttaaagata actgggttgg ggtgcatcca ggtgaaggat tccttggggt tgtagactct |
| 2101 | catccagaag catttgttgg caatttaaac ggaaaaccaa cttacggtaa cacaggtatg |
| 2161 | caaattgcag acgctgcatt ttcatttgat caaacaccag catggagtgt aaattcatta |
| 2221 | acacgtggac agtttaacta ttctggatta caaggtgtta caacttttga tgattcaaaa |
| 2281 | gtatatagta acaaccaaat tgcagacgca ggaagaaaag ttccgaaact tggacttaaa |
| 2341 | ttccaagttg ttggacaggc agatgataaa tcagcaggcg ctgtttggat taaacgttaa |

BAS0638
Accession No. NC_005945
Bacillus anthracis str. Sterne, complete genome.

SEQ ID NO: 52

MRRKAPLKVLSSLAIAAIIGCTSVMSAPLAYAETPAKEKENVSTTPIDYNLIQEDRLA
EALKERGTINPASSKEETKKAVEKYIEKKQGDQANKEILPADTAKEASDFVKKVKE
KKMEEKEKVKKPEKNVSPEQKPEPNKKQLNGQVPTSKAKQAPYKGSVRTDKVLVL
LVEFSDYKHNNIDQTPGYMYSNDFSREHYQKMLFGNEPYTLFDGSKVKTFKQYYE
EQSGGSYTTDGYVTEWLTVPGKASDYGADGSSGHDNKGPKGARDLVKEALHAAA
EKGLDLSQFDQFDRYDTNSDGNQNEPDGVIDHLMVIHAGVGQEAGGGKLGDDAI
WSHRSKLAIDPVAIEGTKSKVDYFGGKVAAHDYTIEPEDGAVGVFAHEFGHDLGLP
DEYDTKYTGTGSPVEAWSLMSGGSWTGKIAGTEPTSFSPQNKDFLQKNMGGNWA
KILEVDYDKIKRGVGVPTYIDQSVTKSNRPGVVRVNLPGKSVETIKPEFGKHAYYST
RGDDMHTLTLETPFFDLTKGTNAKFDYKANYELEAECDFVEVHAVTEDGTKTLIDRL
GEKVVQGDKDTTDGKWIDKSYDLSQFKGKKVKLQFDYITDPAVTYKGFAMDHVN
VTVDGQVVFSDDAEGQSKMNLNGFVVSDGTEKKAHYYYLEWRNYAGSDNGLKA
GKGPVYNTGLVVWYADDSFKDNWVGVHPGEGFLGVVDSHPEAFVGNLNGKPTY
GNTGMQIADAAFSFDQTPAWSVNSLTRGQFNYSGLQGVTTFDDSKVYSNNQIADA
GRKVPKLGLKFQVVGQADDKSAGAVWIKR
BAS0637
Accession No. NC_005945, REGION: 687812 . . . 688459
Bacillus anthracis str. Sterne, complete genome.
Bases 1 to 648
ORIGIN

SEQ ID NO: 53

| | |
|---|---|
| 1 | atgaaattct ttattgatac agcaaatatt aacgaaatta aagaggcaaa tgcattaggc |
| 61 | gtattagctg gagtaacgac aaatccatca cttgtagcaa aagaaggcgt agatttccac |
| 121 | gagcgtattc gtgaaatttg caacgttgta gaaggacctg taagtgcaga agtaattagc |
| 181 | ttagaagcag ataaaatgat cgaagaagga aaagagttag cgaaaattgc tccaaacgtt |
| 241 | gttgtaaagg ttccgatgac aacagaaggt ttaaaagcag taaaagcgtt ctctgactta |
| 301 | ggaattcgta caaacgttac attagtgttc tcagcagttc aagcattact tgcagctcgt |
| 361 | gctggtgcaa catacgtttc accattctta ggtcgcttag atgatatcgg tcataacggt |
| 421 | atggacttaa ttcgccaaat cgcagaaatc tttgcaattc atggcatcga aacagaaatt |
| 481 | atcgcagcat ctgtacgtca cagtgttcac gtaactgacg cagcgttaaa tggtgcacat |
| 541 | attgcaacaa tcccagcaaa cgtaattgct tcattagtga agcatccatt aacagatcaa |
| 601 | ggaattgaga aattcttagc tgattgggaa aaaacacaag agaaataa |

BAS0637
Accession No. NC_005945
Bacillus anthracis str. Sterne, complete genome.

SEQ ID NO: 54

MKFFIDTANINEIKEANALGVLAGVTTNPSLVAKEGVDFHERIREICNVVEGPVSAE
VISLEADKMIEEGKELAKIAPNVVVKVPMYTTEGLKAVKAFSDLGIRTNVTLVFSAV
QALLAARAGATYVSPFLGRLDDIGHNGMDLIRQIAEIFAIHGIETEIIAASVRHSVHV
TDAALNGAHIATIPANVIASLVKHPLTDQGIEKFLADWEKTQEK
BAS1246
Accession No. NC_005945, REGION: 1287792 . . . 1289420
Bacillus anthracis str. Sterne, complete genome.
Bases 1 to 1629
ORIGIN

SEQ ID NO: 55

| | |
|---|---|
| 1 | ttgtttttaa atacgaatga gattcttgat tatagtgcat taaaatatat gccaaatttg |
| 61 | aaatctttaa cagttgcgaa tgcgaagata aaagatccgt cgttctttgc gaacttaaag |
| 121 | caattaaatc atttagcttt gcgtggtaat gaattttcag atgtaacacc acttgttaag |
| 181 | atggatcatt tagattctct tgatttaagt aataataaaa ttacaaacgt tgcaccacta |
| 241 | attgaaatga aaaatgtaaa aagtttatat ttatcaggta accaaataga agatgtaaca |
| 301 | gcattagcga aaatggaaca actagattat ttgaatttag cgaataataa aattacgaat |
| 361 | gttgctccat taagcgcgtt aaaaaatgta acatacttga ctttagctgg taatcaaatt |
| 421 | gaagatatta aaccgttata ttcattacct ttaacagact tagtattaac acgtaataaa |
| 481 | gttaaagatt tatccggcat tgagcaaatg aagcaattag aagaattgtg gatcgggaaa |
| 541 | aatgaaataa aagatgttac tcctctaagt aagatgacac agttaaaaca attacaccta |
| 601 | cctaacaatg agttaaagga tattacgcca ttatcaagtc tagtaaactt acaaaaactt |
| 661 | gatttagaag caaattatat ttcagactta acaccggcta gtaatttgaa aaagttagta |
| 721 | ttcttaagtt ttgttgcaaa tgaaattcgt gatgttcgac cagtgataga actaagtaaa |
| 781 | acagcctaca tcaatgttca aaatcaaaaa gtatttttag aggaaacaga agtaaataaa |
| 841 | gaagtaaaag tacctatata cgaaaaagac ggtaaaatct ctacaaaaat tcgtttgaag |
| 901 | ggcgaaggtg gtacgtatag taacgatgca gttaagtgga gtacaccagg tgagaaagta |
| 961 | tatgaatttg gtgtgaaaga tccatttgcg gatacaggaa tcttctttac gggatctgtc |
| 1021 | attcaaaatg tggtagaaag caaagcggat aacacttcta aagaagacaa tacttctaaa |
| 1081 | gaagatgcaa aagtagaagt agtggaattt aaagatgtac caaaaggaca ttggtcagaa |
| 1141 | gaagcaattc attatttagc gaaagaaaat attttcaagg gatatggaaa tggacaattt |
| 1201 | ggatttgggg atagtattac tcgcggacaa gttgcgtctt tagtacaaag gtacttgaaa |
| 1261 | ttagaaaata aagtagagca gaaagagaga tttacagata cgaaaggaca tatgtttgag |
| 1321 | caagatattg ctacagttgc gcaagctgga attatgcaag gagatggtac tggggagttt |
| 1381 | cgtccagatg gagtattaac tcgatacgaa atgtctgtag tattatataa agtatttcag |
| 1441 | ttaaaagaag atggaaataa taaagtgaac tttaaagatg taccaactgg tcattgggca |
| 1501 | gaagggtatg tgaaagcgtt agtggataat aacatatcaa aaggtgatgg aaaagaacgc |
| 1561 | tttttaggtg atgattttgt aacacgtgaa caatatgcac agtttttata taacgcaata |
| 1621 | acgaaataa |

BAS1246
Accession No. NC_005945
Bacillus anthracis str. Sterne, complete genome.

SEQ ID NO: 56

MFLNTNEILDYSALKYMPNLKSLTVANAKIKDPSFFANLKQLNHLALRGNEFSDVT
PLVKMDHLDSLDLSNNKITNVAPLIEMKNVKSLYLSGNQIEDVTALAKMEQLDYLN
LANNKITNVAPLSALKNVTYLTLAGNQIEDIKPLYSLPLTDLVLTRNKVKDLSGIEQ
MKQLEELWIGKNEIKDVTPLSKMTQLKQLHLPNNELKDITPLSSLVNLQKLDLEAN
YISDLTPASNLKKLVFLSFVANEIRDVRPVIELSKTAYINVQNQKVFLEETEVNKEVK
VPIYEKDGKISTKIRLKGEGGTYSNDAVKWSTPGEKVYEFGVKDPFADTGIFFTGSV
IQNVVESKADNTSKEDNTSKEDAKVEVVEFKDVPKGHWSEEAIHYLAKENIFKGYG
NGQFGFGDSITRGQVASLVQRYLKLENKVEQKERFTDTKGHMFEQDIATVAQAGI
MQGDGTGEFRPDGVLTRYEMSVVLYKVFQLKEDGNNKVNFKDVPTGHWAEGYV
KALVDNNISKGDGKERFLGDDFVTREQYAQFLYNAITK
BAS4444
Accession No. NC_005945, REGION: complement(4354321 . . . 4355034)
Bacillus anthracis str. Sterne, complete genome.
Bases 1 to 714
ORIGIN

SEQ ID NO: 57

| | |
|---|---|
| 1 | atgaaaaagg tttctgtatt acccgctttt attataacgt tcgtatgtat gctagctttt |
| 61 | cttgtaatgc catacgggaa tgcttcagca aaactagctg atggtactta cgatattaat |
| 121 | tacgtaattc aaaaagcgga aaatgattca gcttcaatgg caaatgacta ttttgaaaag |
| 181 | ccagcaaaat taatagtgaa aaacggtgag atgagagtac aagttccgat gaatcatagt |
| 241 | gcttggatta cagaatttaa agcaccagag aacgggaatt ttgttgatgc gaaagttgtt |
| 301 | agtaaagatg aatcggcaga taaaagaaca gtagagttta aagtagatga tttatccaaa |
| 361 | ccggcagctg taaaaattca tgttgttgta ccaaatgcaa actatgacca ccactacaca |
| 421 | attcgttttg cttttgatgc aaatgtaaaa gctgtaggtg gcgataacgg cgtagctgct |
| 481 | acaacaaaaa ataatgatca agcgaaaaca gatacacaag taaaagaaga gaaaacaaaa |
| 541 | gtagagagta aggaaacagc taaagaagtg aacaaagaaa caaaaaatga aaatggaaaa |
| 601 | gctgaaaaaa cagataatcc aaaaacaggc gatgaagcac gtattggatt gtttgcagcg |
| 661 | ttaattctta tttcaggtgt tttcttaatt cgtaaagtga aattgagtaa ataa |

BAS4444
Accession No. NC_005945
Bacillus anthracis str. Sterne, complete genome.

SEQ ID NO: 58

MKKVSVLPAFIITFVCMLAFLVMPYGNASAKLADGTYDINYVIQKAENDSASMAN
DYFEKPAKLIVKNGEMRVQVPMNHSAWITEFKAPENGNFVDAKVVSKDESADKRT
VEFKVDDLSKPAAVKIHVVVPNANYDHHYTIRFAFDANVKAVGGDNGVAATTKN
NDQAKTDTQVKEEKTKVESKETAKEVNKETKNENGKAEKTDNPKTGDEARIGLFA
ALILISGVFLIRKVKLSK
BAS4444
Accession No. NC_005945, REGION: complement(4354321 . . . 4355034)
Bacillus anthracis str. Sterne, complete genome.
Bases 1 to 1287
ORIGIN

SEQ ID NO: 59

| | |
|---|---|
| 1 | atgtttatag atcaggtcaa gatatatgta aaaggcggcg acggtggtaa cggaatggtt |
| 61 | gcgtatcgtc gtgagaagta tgtaccaaaa ggtggcccag caggtggcga cggtggtaaa |
| 121 | ggtgcagatg ttgtttttat tgttgaggaa ggcttacgta cattaatgga cttccgctac |
| 181 | caacgtcatt tcaaagctga tcgcggtcag cacggaatga gtaaaggtca gcacggacgt |
| 241 | aaatctgaag atttacttgt aaaggttccg ccaggaacag tagtaaaaga tgaaaaaact |
| 301 | ggtcaaattc ttgcggattt agtaacgcat ggacaaacgg ctgtaattgc aaaaggtggc |
| 361 | cgcggtggtc gtggtaactc acgtttcgca acagctacga acccagcgcc agaaatcgct |
| 421 | gaaaacgggg aaccaggtca agagcgtgat gtcattctag aactgaaagt actggcagac |
| 481 | gttggacttg ttggattccc aagtgtaggt aaatctacat tattatctgt cgtatcatca |
| 541 | gcacgtccga aaattgcaga gtatcacttc acaacaatcg ttccaaacct tggtgttgtt |
| 601 | gaaactggtg ataaccgcag cttcgttatg gctgaccttc ctggactaat tgaaggcgca |
| 661 | catgctggcg tcggacttgg acaccaattc ttacgtcata ttgagcgtac acgtgtaatc |
| 721 | gtgcatgtta ttgatatgtc tggtttagaa ggccgtgatc catatgaaga ttacgtaaca |
| 781 | attaataatg aattaaaaga atacaatctt cgcttaacgg agcgtccaca agttgttgtt |
| 841 | gcaaacaaaa tggatatgcc agatgcagaa gaaaacttac aagcatttaa agagaaagtg |
| 901 | ggagacgaag taaaaatctt cccaatctca gctgtaacga aacaaggtgt tcgtgactta |
| 961 | ctgtttgaag tagcgaactt aatagaaaca acacctgaat tcccaataca tgaagttgtg |
| 1021 | gatgagtctg acacaagtgt aatgtacaaa tttgagactg aaggtgttaa atttgatatt |
| 1081 | acacgtgaaa gtgatggtac gtttgttatc tctggttacg atatcgagaa aacattcaag |
| 1141 | atgacagact tctcacgtga tgaatctgta cgtcgtttcg ctcgccaaat gcgcggaatg |
| 1201 | ggtattgatg aagcgcttcg tgcacgtggt gcaaaagacg gagatattgt aaaaattctt |
| 1261 | gaatatgaat ttgaatttat cgactaa |

BAS4444
Accession No. NC_005945
Bacillus anthracis str. Sterne, complete genome.

SEQ ID NO: 60

MFIDQVKIYVKGGDGGNGMVAYRREKYVPKGGPAGGDGGKGADVVFIVEEGLRT
LMDFRYQRHFKADRGQHGMSKGQHGRKSEDLLVKVPPGTVVKDEKTGQILADLV
THGQTAVIAKGGRGGRGNSRFATATNPAPEIAENGEPGQERDVILELKVLADVGLV
GFPSVGKSTLLSVVSSARPKIAEYHFTTIVPNLGVVETGDNRSFVMADLPGLIEGAH
AGVGLGHQFLRHIERTRVIVHVIDMSGLEGRDPYEDYVTINNELKEYNLRLTERPQV
VVANKMDMPDAEENLQAFKEKVGDEVKIFPISAVTKQGVRDLLFEVANLIETTPEF
PIHEVVDESDTSVMYKFETEGVKFDITRESDGTFVISGYDIEKTFKMTDFSRDESVRR
FARQMRGMGIDEALRARGAKDGDIVKILEYEFEFID
BAS4236
Accession No. NC_005945, REGION: complement(4150337 . . . 4151050)
Bacillus anthracis str. Sterne, complete genome.
Bases 1 to 714
ORIGIN

SEQ ID NO: 61

| | |
|---|---|
| 1 | ttgagtctat tcgccgcaat tggatatatg gttcgagaag tgtttgtttt tgtttcttat |
| 61 | gtgaagaaca atgcgtttcc gcagccatta tcatcagacg atgagagaaa gtacttagag |
| 121 | ttaatggagc aaggtgatgc tcaagcgagg aatctgttaa ttgaacataa tttacggctt |
| 181 | gtagctcata tcgttaaaaa atttgaaaat acaggggaag atgcagaaga tttaatttca |
| 241 | attggtacaa tcgggctcat taaagcaatc gagagctatt cggcaggaaa aggtacaaaa |
| 301 | cttgcgacgt acgcagcacg ctgtattgaa aatgaaattt tgatgcattt acgtgtatta |
| 361 | aagaaaacga aaaaggacgt ttcacttcat gatccaatcg ggcaagataa agaggggaat |
| 421 | gaaatatcgc ttattgatat attaaaatca gagtctgaag atgtaattga catgatccag |
| 481 | cttagtatgg agttagaaaa gattaaagag tatatcgata ttttagacga acgagagaaa |
| 541 | gaagtaatcg tgaagcgttt tggactgggg cttgataagg agaagacgca acgagagatt |
| 601 | gcgaaggcac ttggtatttc cagaagctat gtatcaagaa ttgaaaagcg cgctttaatg |
| 661 | aaaatgttcc atgaatttgt aagagcagag aaagagaaaa aagcaaaaga ataa |

BAS4236
Accession No. NC_005945
Bacillus anthracis str. Sterne, complete genome.

SEQ ID NO: 62

MSLFAAIGYMVREVFVFVSYVKNNAFPQPLSSDDERKYLELMEQGDAQARNLLIE
HNLRLVAHIVKKFENTGEDAEDLISIGTIGLIKAIESYSAGKGTKLATYAARCIENEIL
MHLRVLKKTKKDVSLHDPIGQDKEGNEISLIDILKSESEDVIDMIQLSMELEKIKEYI
DILDEREKEVIVKRFGLGLDKEKTQREIAKALGISRSYVSRIEKRALMKMFHEFVRA
EKEKKAKE
BAS4413
Accession No. NC_005945, REGION: complement(4321621 . . . 4323414)
Bacillus anthracis str. Sterne, complete genome.
Bases 1 to 1794
ORIGIN

SEQ ID NO: 63

| | |
|---|---|
| 1 | atgaaaggga aacttatagt agtcggcggt ggcttggctg gcttaatggc aacgattaaa |
| 61 | gcggcggaag caggagtaaa tgttgaactg ttctctttag taccagtaaa acgttcgcat |
| 121 | tctgtatgtg cccaaggtgg aattaacggt gccgtgaata cgaaaggtga aggggattct |
| 181 | ccatggatcc actttgacga tacaatttat ggtggggact tcttagcgaa ccaaccacca |
| 241 | gttaaagcaa tgtgtgaagc agcacctggt atcattcatt taatggaccg tatgggtgtt |
| 301 | atgttcaacc gtacggaaga aggacttctt gatttccgtc gttttggtgg aacgcaacat |
| 361 | caccgtacag catttgctgg tgcaacaact ggacagcaat tactatacgc attagatgag |
| 421 | caagtacgtc gtcatgaagt agcaggactt gtaacgaaat atgaaggttg ggatttctta |
| 481 | cgagctgttg ttgatgacga aggtgtgtgc cgaggaatcg ttgcacaaga cttacaaaca |
| 541 | atggagatta aaagtttcgg agctgatgcc gtgattatgg caacaggggg ccctggtatc |
| 601 | atcttcggaa aatcaactaa ctctattatt aatacaggta cagcagcttc tgctgtatat |
| 661 | caacaaggcg catattatgc aaacggtgag ttcattcaaa ttcacccaac ggcaattcct |
| 721 | ggagacgata aattacgtct tatgagtgaa tctgcacgtg gtgaaggtgg acgtgtttgg |
| 781 | acatataaag atggtaaacc atggtacttc ttagaagaaa aatatccggc ttacggaaat |
| 841 | cttgtacctc gtgatatcgc aacgcgtgaa atctttgatg tttgcgtaga gcaaaaacta |
| 901 | ggtattaacg gtgaaaatat ggtttactta gatctttctc ataaagatcc gaaagaacta |
| 961 | gatattaaac ttggtggaat tattgaaatc tatgagaaat ttacaggtga tgatcctcgt |
| 1021 | aaactaccaa tgaaaatctt cccagctgtt cactattcaa tgggcggact atgggttgat |
| 1081 | tataaacaga tgacaaatat tccaggttta tttgcagcag gtgagtgtga ttattctatg |
| 1141 | cacggtggta accgtcttgg tgcgaactca ctattatcag caatttacgg tggtatggta |
| 1201 | gcaggaccga atgcaattga atatatgaaa ggtctttcta aatcatcaga tgctgtttca |
| 1261 | tctactgtgt atgaacaaaa tgaattaatc gaaacagaga aatttaacaa tattttaacg |
| 1321 | ctcgatggta acgaaaatgc gtatgttctt cataaagagc ttggagaatg gatgacagat |
| 1381 | aacgttacag tagttcgtga aaataaaaaa ttattagaaa cagatgcaaa aattgaagag |
| 1441 | ttaatggctc gttataaacg tattaacatt aacgatacag caagatggag taaccaaggt |
| 1501 | gcttcattta cacgccaact tgcaaatatg tttgagctag cacgtgttat tacaattggt |
| 1561 | gcatataacc gtaatgagag ccgtggggcg cattacaaac ctgaattccc aaatcgtgat |
| 1621 | gatgcaaact tcttaaaaac tacgatggca aaatttgaag gagaaggaaa tgcaccagca |
| 1681 | ttccattatg aagatgtgga tatttcatta attaaaccac gtaaacgtga ttattcttca |
| 1741 | aaacacgatg tagctgctaa gggtgaagag aagggggata aacaacatgt ctga |

BAS4413
Accession No. NC_005945
Bacillus anthracis str. Sterne, complete genome.

SEQ ID NO: 64

MKGKLIVVGGGLAGLMATIKAAEAGVNVELFSLVPVKRSHSVCAQGGINGAVNTK
GEGDSPWIHFDDTIYGGDFLANQPPVKAMCEAAPGIIHLMDRMGVMFNRTEEGLL
DFRRFGGTQHHRTAFAGATTGQQLLYALDEQVRRHEVAGLVTKYEGWDFLRAVV
DDEGVCRGIVAQDLQTMEIKSFGADAVIMATGGPGIIFGKSTNSIINTGTAASAVYQ
QGAYYANGEFIQIHPTAIPGDDKLRLMSESARGEGGRVWTYKDGKYWYFLEEKYP
AYGNLVPRDIATREIFDVCVEQKLGINGENMVYLDLSHKDPKELDIKLGGIIEIYEKF
TGDDPRKLPMKIFPAVHYSMGGLWVDYKQMTNIPGLFAAGECDYSMHGGNRLGA
NSLLSAIYGGMVAGPNAIEYMKGLSKSSDAVSSTVYEQNELIETEKFNNILTLDGNE
NAYVLHKELGEWMTDNVTVVRENKKLETDAKIEELMARYKRININDTARWSNQ
GASFTRQLANMFELARVITIGAYNRNESRGAHYKPEFPNRDDANFLKTTMAKFEGE
GNAPAFHYEDVDISLIKPRKRDYSSKHDVAAKGEEKGDKQHV
BAS2768
Accession No. NC_005945, REGION: 2748965 . . . 2750131
Bacillus anthracis str. Sterne, complete genome.
Bases 1 to 1167
ORIGIN

SEQ ID NO: 65

| | |
|---|---|
| 1 | ttgttaatgt ccaatttatt agctttcgat gctggtacag gaagtattcg agcagttctt |
| 61 | tttgatttac atggtaatca aattgcagta agtcaaaaag aatggattca caaatctgac |
| 121 | ccactttatc caggctctat gaactttgat gtaatagaaa attggaaact agtacaagaa |
| 181 | tgcacgaaag aggttcttca aaaaagcaat actcttgcct cttccattct tgctattagt |
| 241 | gcgacaagta tgcgggaagg gtttgtttta tatgatcaag atgggcaaga aatatgggcg |
| 301 | tgtgcaaatg ttgatgggcg cgcatctgct gaggttagtg aactaaaaga aattcggtca |
| 361 | caccttgaaa aagatttata tacaaagtct ggtcaaactt tttcattagg tgctttaccc |
| 421 | cgcctacttt ggattaaaaa acatgaaccc gacgtctata atactattca ctcttttaca |
| 481 | atgttaaacg attggatctt atataaatta agcagagtgc tgcaaatcgg tccttcaaac |
| 541 | ggatgtacgt caggtatttt tgacttacaa aatagagtgt gggataacga tgttgctaag |
| 601 | gagtgcggtc tatctttacc attttcacca acagtaaatg aagctggtac agtaattggc |
| 661 | aacgttacaa aagagtgtgc agcattaact ggattatgtg aaggaattcc tgtcgtagcc |
| 721 | gggggcggag atgctcaaat ggcatcgctc ggaactggag tcgtgaaacc gaatcaaaca |
| 781 | ttaatatgcg gaggtagttt ttggcaacaa gaagcgaatg ttactgagcc aataccagat |
| 841 | ccacaagctg cgattcgtat aaattgtcac gtcgtacgta acctatggca atacgaaacg |
| 901 | attgcctttt tcccaggcct cgttatgcgc tggtttcgag acgctttttg tcaagaggaa |
| 961 | aagaaacttg ctgacaaact cggtgtagat gcttatgaat tactagaaga acaagcgaaa |
| 1021 | gacgtacctg tcggttcaca tggcattatc cctacttttt caaacgttat gaactacatt |
| 1081 | tcttggcgtc atgccgcacc ttctttttta aatttaagtt tagacgctga caaatgcgga |
| 1141 | aaaaaaagaa ctgtttcgtg ctattga |

BAS2768
Accession No. NC_005945
Bacillus anthracis str. Sterne, complete genome.

SEQ ID NO: 66

MLMSNLLAFDAGTGSIRAVLFDLHGNQIAVSQKEWIHKSDPLYPGSMNFDVIENWK
LVQECTKEVLQKSNTLASSILAISATSMREGFVLYDQDGQEIWACANVDGRASAEV
SELKEIRSHLEKDLYTKSGQTFSLGALPRLLWIKKHEPDVYNTIHSFTMLNDWILYK
LSRVLQIGPSNGCTSGIFDLQNRVWDNDVAKECGLSLPFSPTVNEAGTVIGNVTKEC
AALTGLCEGIPVVAGGGDAQMASLGTGVVKPNQTLICGGSFWQQEANVTEPIPDPQ
AAIRINCHVVRNLWQYETIAFFPGLVMRWFRDAFCQEEKKLADKLGVDAYELLEE
QAKDVPVGSHGIIPTFSNVMNYISWRHAAPSFLNLSLDAKCGKKRTVSCY
BAS0672
Accession No. NC_005945, REGION: 724379 . . . 725554
Bacillus anthracis str. Sterne, complete genome.
Bases 1 to 1176
ORIGIN

SEQ ID NO: 67

| | |
|---|---|
| 1 | atggatgttg caaaagaact tgttttgtca aaggatcagt tggttgagtg gagaaggcat |
| 61 | tttcataagt atccagagtt atcttttcaa gaagaaaaaa catcacagtt tgtattcgac |
| 121 | atacttcgga aaatcccatg tttagaagtg tcaagaccta ctaaatatag tgtaatggca |
| 181 | aggttgattg gaaagcagtc tggtaaaact attgcggttc gagctgacat ggatgctctt |
| 241 | cctattcatg aagaaaatga gtttgacttt atttctgcat atccaggtgt aatgcatgca |
| 301 | tgtggccatg atggacatat agcgatatta cttggagtcg tacataagtt agtagaagca |
| 361 | agagagaaga ttaaaggaga ggttcgtttt ctattccagc atgctgaaga aaactttcct |
| 421 | ggcggagcag aggaaatggt cgcggctggt gtaatggaag gggtcgatta tattgttggt |
| 481 | gctcaccttt gggcgtcatt agaggttggg aaagtaggtg taatttatgg tcctgcaatg |
| 541 | gctgccccag atgtttttaa aattacgata gaaggaaaag gtggacatgc tggaatccct |
| 601 | catgaaacgg ttgatagtat tgccatcggc acacaagtcg tttcacaact tcagcaaatt |
| 661 | gtatctcgtc tcacgaatcc gttagattct ctcgtagtat ctgttacgca atttcatgct |
| 721 | gggacaaccc ataatgtaat tccagcacaa acggagattg aagggacagt gcggagttta |
| 781 | agacatgagt tacgagaaga aacagagaaa aggattgaac agattgtaaa gcatgtgacg |
| 841 | gaagcttatg gagcgaaata tactttttct tatgaatatg gatatcgtcc agtagtaaat |
| 901 | gattatgaag tgacagagat tattgagcaa acagcattac agctttatgg aagggaacga |
| 961 | gttactcgtt tacagccgac gatggctgga gaagattttt cagcgttttt acaaaaagta |
| 1021 | ccagggacat tcttttttat cggagcagga agtaaagaga aaggaattat atatcctcat |
| 1081 | catcaccctc gttttacgat tgatgaagat gcattaccaa ttggcgtgca agtctttgta |
| 1141 | tcatcgatta tgaatttcat aagtaaagga gaatga |

BAS0672
Accession No. NC_005945
Bacillus anthracis str. Sterne, complete genome.

SEQ ID NO: 68

MDVAKELVLSKDQLVEWRRHFHKYPELSFQEEKTSQFVFDILRKIPCLEVSRPTKYS
VMARLIGKQSGKTIAVRADMDALPIHEENEFDFISAYPGVMHACGHDGHIAILLGV
VHKLVEAREKIKGEVRFLFQHAEENFPGGAEEMVAAGVMEGVDYIVGAHLWASLE
VGKVGVIYGPAMAAPDVFKITIEGKGGHAGIPHETVDSIAIGTQVVSQLQQIVSRLT
NPLDSLVVSVTQFHAGTTHNVIPAQTEIEGTVRSLRHELREETEKRIEQIVKHVTEAY
GAKYTFSYEYGYRPVVNDYEVTEIIEQTALQLYGRERVTRLQPTMAGEDFSAFLQK
VPGTFFFIGAGSKEKGIIYPHHHPRFTIDEDALPIGVQVFVSSIMNFISKGE
BAS0673
Accession No. NC_005945, REGION: 725556 . . . 725753
Bacillus anthracis str. Sterne, complete genome.
Bases 1 to 198
ORIGIN

SEQ ID NO: 69

| | |
|---|---|
| 1 | atgaagaaaa tacacgtatt agcgcttatt ccagtttttt gtttagttgt tggccccgta |
| 61 | tttgctaatt cagttactcc ttacatatta gggatgccat ttttattatt ttggatatta |
| 121 | ttatcagtgc ttattacatc tctttgtatg gggattgtat acgtatttga tcctgctaat |
| 181 | aagggggatg taaaatga |

BAS0673
Accession No. NC_005945
Bacillus anthracis str. Sterne, complete genome.

SEQ ID NO: 70

MKKIHVLALIPVFCLVVGPVFANSVTPYILGMPFLLFWILLSVLITSLCMGIVYVFDP
ANKGDVK
BAS4315
Accession No. NC_005945, REGION: complement(4227217 . . . 4228227)
Bacillus anthracis str. Sterne, complete genome.
Bases 1 to 1011
ORIGIN

SEQ ID NO: 71

| | |
|---|---|
| 1 | gtgagtataa tggacgaacg tctcctttca ggggaatctg catatgaaga tgcggattta |
| 61 | gaatattcat tacggccaca gacgctccgt cagtatattg gccaagataa agcgaaacat |
| 121 | aatttagaag tgtttattga agcggcgaaa atgcgtgaag aaacgttaga tcacgtgctt |
| 181 | ttatatggac caccaggact tggtaaaacg acgcttgcga atattattgc caatgaaatg |
| 241 | ggcgtaaatg ttagaacaac ttcaggtcca gcaatcgaaa ggccaggaga tttagcagct |
| 301 | gtattaacat cgcttcaacc aggggatgta ttatttattg atgaaattca tcgtttgcat |
| 361 | agatcaattg aagaagtact atatcctgcg atggaagatt tttgccttga tattgtcatt |
| 421 | ggaaaaggac cgtcagcgcg atctgtacgt ttagatttac cgccatttac attagttgga |
| 481 | gcaacgacgc gtgcgggagc attatcagcg ccattacgtg accgtttcgg tgtactttca |
| 541 | agattagagt attacacagt agatcagctt tctgcgattg tggaacgtac agcagaagta |
| 601 | tttgaagttg aaattgattc gttagctgca ctagaaattg caagacgtgc tcgtggtaca |
| 661 | cctcgtattg cgaatcgttt attacgacgt gtacgagatt tcgcacaagt tcgtggtaac |
| 721 | ggaacagtta cgatggaaat tacgcaaatg gcattagaat tgctgcaagt agataaatta |
| 781 | ggtctagatc atattgacca taaattattg cttggtatta ttgaaaaatt ccacggtggc |
| 841 | ccagttggac tagaaacggt ttcggcaacg attggagaag aatctcatac gattgaagat |
| 901 | gtgtatgagc catatttatt acaaattggc tttttacaac gaacgccaag gggccggatt |
| 961 | gtaacgccgc ttgcatatga gcatttcgga atggagatgc caaaagtatg a |

BAS4315
Accession No. NC_005945
Bacillus anthracis str. Sterne, complete genome.

SEQ ID NO: 72

MSIMDERLLSGESAYEDADLEYSLRPQTLRQYIGQDKAKHNLEVFIEAAKMREETL
DHVLLYGPPGLGKTTLANIIANEMGVNVRTTSGPAIERPGDLAAVLTSLQPGDVLFI
DEIHRLHRSIEEVLYPAMEDFCLDIVIGKGPSARSVRLDLPPFTLVGATTRAGALSAP
LRDRFGVLSRLEYYTVDQLSAIVERTAEVFEVEIDSLAALEIARRARGTPRIANRLLR
RVRDFAQVRGNGTVTMEITQMALELLQVDKLGLDHIDHKLLLGIIEKFHGGPVGLE
TVSATIGEESHTIEDVYEPYLLQIGFLQRTPRGRIVTPLAYEHFGMEMPKV
BAS5149
Accession No. NC_005945, REGION: complement(5033136 . . . 5033675)
Bacillus anthracis str. Sterne, complete genome.
Bases 1 to 540
ORIGIN

SEQ ID NO: 73

| | |
|---|---|
| 1 | ttggaaaaag aaggtgttac aatggttata aattttgagg aattacatcc aaatgagcga |
| 61 | gcggaattag aacgaaatat ctttttttct acattggaac agttgaaagg atgggcgcga |
| 121 | agtaattctt tatggccgat gacattcgga ctggcatgct gtgcaattga aatgatggga |
| 181 | gtaggttcat cacattatga tttagatcga tttgggtcat tttttcggac ttcaccaagg |
| 241 | caatcggacg ttatgattgt gtcgggaacg gtaacgaaga agatggctcc tattgttcgg |
| 301 | cgcttatatg accaaatgcc tgaaccaaaa tgggttattg caatgggatc ttgtgcgaca |
| 361 | gcaggtggtc catatgtaaa ttcgtacgct gttgtgaaag gtgtagatca aattgtgcca |
| 421 | gttgacgtgt atatccctgg ttgcccacca aatcctgcag ctttaattta tggaattaat |
| 481 | aaattaaaag aaaaaattcg ttacgaagca aagactggaa agcaggtgac gaataaatga |

BAS5149
Accession No. NC_005945
Bacillus anthracis str. Sterne, complete genome.

SEQ ID NO: 74

MEKEGVTMVINFEELHPNERAELERNIFFSTLEQLKGWARSNSLWPMTFGLACCAI
EMMGVGSSHYDLDRFGSFFRTSPRQSDVMIVSGTVTKKMAPIVRRLYDQMPEPKW
VIAMGSCATAGGPYVNSYAVVKGVDQIVPVDVYIPGCPPNPAALIYGINKLKEKIRY
EAKTGKQVTNK
BAS4186
Accession No. NC_005945, REGION: complement(4101517 . . . 4102413)
Bacillus anthracis str. Sterne, complete genome.
Bases 1 to 897
ORIGIN

SEQ ID NO: 75

| | |
|---|---|
| 1 | atgttaaaga ttggatctca tgtttccatg agcgggaaga aaatgttatt agcagcaagt |
| 61 | gaagaggctg tttcatacgg tgcaacaacg tttatgattt atacaggtgc accgcaaaat |
| 121 | acaagaagaa aaccaattga agaattgaac atagaagcag gaagaaaaca tatggaacaa |
| 181 | aacggtattg aagagattat cgtacatgcg ccatatatta ttaatgtcgg aaatacgacg |
| 241 | aagccagaaa cattccaatt aggtgtagat ttccttcgta tggaaattga gagaacatca |
| 301 | gcattaggtg tggcgaaaca aatcgttctt cacccaggtg cgcacgttgg tgcaggagcg |
| 361 | gatgctggta ttcaacagat tattaaagga cttaatgaag tgttaacgcc agatcagact |
| 421 | gttaacattg cgttagaaac gatggcagga aaaggaacag aatgcggccg tagtttcgag |
| 481 | gaaattgcaa aaattattga tggcgtaaaa tataatgaaa aactatcagt atgctttgat |
| 541 | acatgtcata cgcacgatgc aggatatgac attgtaaata actttgacgg tgtattaaac |
| 601 | gaatttgata agattgttgg tatcgatcgt ttacaagtac ttcatattaa tgatagtaaa |
| 661 | aatgtacgcg gcgcaggaaa agaccgtcat gaaaatattg gtttcggtca tatcggttat |
| 721 | aaagcattgc atcatattgt acatcatcca cagttaacgc acgtaccaaa aattcttgaa |
| 781 | acgccatatg taggtgaaga taaaaaagat aagaagccgc catataaatt agaaatcgaa |
| 841 | atgctgaaaa atggtacttt tgatgaagga cttcttgaaa aaattaaagc gcaataa |

BAS4186
Accession No. NC_005945
Bacillus anthracis str. Sterne, complete genome.

SEQ ID NO: 76

MLKIGSHVSMSGKKMLLAASEEAVSYGATTFMIYTGAPQNTRRKPIEELNIEAGRK
HMEQNGIEEIIVHAPYIINVGNTTKPETFQLGVDFLRMEIERTSALGVAKQIVLHPGA
HVGAGADAGIQQIIKGLNEVLTPDQTVNIALETMAGKGTECGRSFEEIAKIIDGVKY
NEKLSVCFDTCHTHDAGYDIVNNFDGVLNEFDKIVGIDRLQVLHINDSKNVRGAGK
DRHENIGFGHIGYKALHHIVHHPQLTHVPKILETPYVGEDKKDKKPPYKLEIEMLKN
GTFDEGLLEKIKAQ
BAS4875
Accession No. NC_005945, REGION: complement(4753280 . . . 4755064)
Bacillus anthracis str. Sterne, complete genome.
Bases 1 to 1785
ORIGIN

SEQ ID NO: 77

| | |
|---|---|
| 1 | atggaaaaaa cagtaggaaa tgcggttaaa ggcggtagct ttttagtaga tgagattacg |
| 61 | attgatcaag tgtttacgcc agaagatttt tcatctgagc ataaaatgat tgcaaaaacg |
| 121 | acagaggact ttatcgtaaa tgaagttctt ccagagcttg aatatttaga gcaacatgag |
| 181 | tttgatcgtt ctgttcgtct tttaaaagaa gctggggaac ttggtttatt aggcgctgac |
| 241 | gtaccagaag agtacggcgg aattggtctt gataaagtaa gctcagcgtt aatcgcagag |
| 301 | aaattctctc gcgctggtgg ttttgcaatt actcacggtg ctcacgtagg tatcggatct |
| 361 | ttaccaatcg tgttattcgg taacgaagag caaaagaaaa agtatttacc attgcttgca |
| 421 | actggtgaaa aattagctgc atacgcatta acagagccag gttcaggatc tgacgcacta |
| 481 | ggtgcaaaaa caacagcacg tttaaatgca gaaggtacac attacgtatt aaatggtgaa |
| 541 | aaacagtgga ttacaaactc tgcattcgct gacgtattta tcgtatacgc aaaaattgat |
| 601 | ggagagcact tctcagcatt tatcgtagag aaagactacg ctggtgtatc tacaagccca |
| 661 | gaagaaaaga aaatgggtat taaatgttct tcaactcgta cgttaatttt agaagatgcg |
| 721 | ttagtaccga aagaaaactt acttggtgaa atcggtaaag ggcatattat cgcattcaac |
| 781 | attttaaata tcggtcgtta taaattaggt gttggtacag ttggatctgc gaaacgtgca |
| 841 | gtagaaattt cagcacaata tgcaaaccaa cgtcaacagt tcaaacaacc aatcgctcgc |
| 901 | ttcccattaa ttcaagagaa acttgcgaat atggcagcga aaacatatgc agctgaaagc |
| 961 | tctgtatatc gtacagtagg tttattcgaa agccgcatga gcacattatc tgaagaagaa |
| 1021 | gtaaaagacg gtaaagcagt tgcagcttct atcgctgaat atgcaatcga gtgctcttta |
| 1081 | aacaaagtat tcggttctga agtactagac tatacagtag atgaaggtgt tcaaatccac |
| 1141 | ggtggttacg gatttatggc agagtacgag attgaaagaa tgtatcgcga ttctcgtatt |
| 1201 | aaccgtattt tcgaaggaac gaatgaaatt aaccgcctaa tcgtaccagg tacgttctta |
| 1261 | cgtaaagcga tgaaaggtga attaccactt cttcaaaagg cacaaaaatt acaagaagag |
| 1321 | ttaatgatga tgatgccaga agaagtaggc gatgagccat tagcacttca aaaatattta |
| 1381 | gtaaataacg cgaagaaaat cggcttaatg gtagctggat tagctgctca aaaatacggt |
| 1441 | aaagcattag ataaagagca agaaattctt gtgaatatcg ctgacatcgt aagtaaccta |
| 1501 | tacgcaatgg aatcagctgt tcttcgtaca gaaaaagcaa ttaaaacaac tggtcttgaa |
| 1561 | aagaataaac aaaaagtgtt atacactgaa gtattctgcc aagaagcgtt caacgaaatc |
| 1621 | gaagcacatg cgaaagaaac acttatcgca gttgaaaacg gcgacatgtt acgcatgatg |
| 1681 | ttatcatcat tacgtaaatt aactcgccac acaccactta acgtaattcc gaagaagcgt |
| 1741 | gaaatcgctg cgaaaatttt agaagatgag cgttatacag tttaa |

BAS4875
Accession No. NC_005945
Bacillus anthracis str. Sterne, complete genome.

SEQ ID NO: 78

MEKTVGNAVKGGSFLVDEITIDQVFTPEDFSSEHKMIAKTTEDFIVNEVLPELEYLE
QHEFDRSVRLLKEAGELGLLGADVPEEYGGIGLDKVSSALIAEKFSRAGGFAITHGA
HVGIGSLPIVLFGNEEQKKKYLPLLATGEKLAAYALTEPGSGSDALGAKTTARLNA
EGTHYVLNGEKQWITNSAFADVFIVYAKIDGEHFSAFIVEKDYAGVSTSPEEKKMGI
KCSSTRTLILEDALVPKENLLGEIGKGHIIAFNILNIGRYKLGVGTVGSAKRAVEISAQ
YANQRQQFKQPIARFPLIQEKLANMAAKTYAAESSVYRTVGLFESRMSTLSEEEVK
DGKAVAASIAEYAIECSLNKVFGSEVLDYTVDEGVQIHGGYGFMAEYEIERMYRDS
RINRIFEGTNEINRLIVPGTFLRKAMKGELPLLQKAQKLQEELMMMMPEEVGDEPL
ALQKYLVNNAKKIGLMVAGLAAQKYGKALDKEQEILVNIADIVSNLYAMESAVLR
TEKAIKTTGLEKNKQKVLYTEVFCQEAFNEIEAHAKETLIAVENGDMLRMMLSSLR
KLTRHTPLNVIPKKREIAAKILEDERYTV
BAS4876
Accession No. NC_005945, REGION: complement(4755339 . . . 4756511)
Bacillus anthracis str. Sterne, complete genome.
Bases 1 to 1173
ORIGIN

SEQ ID NO: 79

| | |
|---|---|
| 1 | atgagagaag ctgtcattgt tgcgggagca agaacaccaa ttggaaaagc aaagaggggt |
| 61 | tcattaaaaa cagttcgtcc tgacgatcta ggggcgttag tagtaaagga aacgttaaag |
| 121 | cgtgcgaatt atgaaggacc aatcgatgat ttaattttcg gttgtgcgat gccagaagca |
| 181 | gagcaaggtt taaatatggc tcgtaatatc ggcggattag caggactttc ttacgatgtt |
| 241 | ccagctatta caattaaccg ttactgttct tcaggtttac aaagtatcgc ttacggagca |
| 301 | gagcgcatta tgcttggtca ctcggaagcg gtattatcag gcggagcgga atcaatgagt |
| 361 | ttagttccga tgatgggaca cgtcgttcgt ccgaatagtc gccttgtaga agcggctcca |
| 421 | gaatattata tgggtatggg acatacagcg gagcaagttg ctgtgaaata tggaatttct |
| 481 | cgtgaagagc aagatgcatt tgcagtaaga agtcatcaac gtgctgcgaa agcattagct |
| 541 | gcaggaaact ttgctgatga aacagtatct gtagatgtaa cgttacgtac tgttggagca |
| 601 | aataacaaac tgcaagaaga aacaattact tttgcgcaag acgaaggtgt aagagcagag |
| 661 | acgacgctag atattttagg taaattacgt ccagcattta acgttcgcgg ttctgtaaca |
| 721 | gctggtaact cttcacaaat gagtgacggt gcagcatctg tactattaat ggatcgtgaa |
| 781 | aaagcagtga gcgatggcat gaaaccactt gcgaaattcc gttcatttgc agtagctggc |
| 841 | gtaccaccag aagtaatggg aatcggccca atcgctgcca ttccaaaagc gttaaaacta |
| 901 | gctggcttag agctatctga tattggctta tttgaactaa atgaagcatt cgcttctcaa |
| 961 | tcgatccaag ttattcgtga acttggttta gatgaagaaa aagtaaacgt aaacggcggt |
| 1021 | gcaatcgcac ttggacatcc acttggctgt acaggagcaa aactaacgct atctcttatt |
| 1081 | cacgaaatga aacgccgcaa cgaacaattc ggtatcgtaa caatgtgtat cggcggcgga |
| 1141 | atgggagcag caggagtgtt tgaattacta taa |

BAS4876
Accession No. NC_005945
Bacillus anthracis str. Sterne, complete genome.

SEQ ID NO: 80

MREAVIVAGARTPIGKAKRGSLKTVRPDDLGALVVKETLKRANYEGPIDDLIFGCA
MPEAEQGLNMARNIGGLAGLSYDVPAITINRYCSSGLQSIAYGAERIMLGHSEAVLS
GGAESMSLVPMMGHVVRPNSRLVEAAPEYYMGMGHTAEQVAVKYGISREEQDAF
AVRSHQRAAKALAAGNFADETVSVDVTLRTVGANNKLQEETITFAQDEGVRAETT
LDILGKLRPAFNVRGSVTAGNSSQMSDGAASVLLMDREKAVSDGMKPLAKFRSFA
VAGVPPEVMGIGPIAAIPKALKLAGLELSDIGLFELNEAFASQSIQVIRELGLDEEKV
NVNGGAIALGHPLGCTGAKLTLSLIHEMKRRNEQFGIVTMCIGGGMGAAGVFELL
BAS2724
Accession No. NC_005945, REGION: complement(2701773 . . . 2703020)
Bacillus anthracis str. Sterne, complete genome.
Bases 1 to 1248
ORIGIN

SEQ ID NO: 81

| | |
|---|---|
| 1 | atgtatacag aacaagaaca gaaagattat atgaaagtat ggttttcact tgcagaagag |
| 61 | gctggtcgga atgggtttac ttggccgagt ctattagaaa atgaagaatg gaatcaatat |
| 121 | atggcaacag gtatgtatcg tatgccaata aaaacttata ctgctatttc aaaagcgaca |
| 181 | gaagaaatta tgtatgtgct atatagaacg tatcaatata ttctcaatac gtcaaaagat |
| 241 | tttcaaaaac taggatttcc agctgaaacg tgggaaattg cgagaatgaa acatactggt |
| 301 | ttgttttcat attttacaag gtttgacttt atcgtaaata gtgaagatat aaagttaata |
| 361 | gaagtaaatt gtgatacacc aacaggttat ttagaaccgt ctgtcgcaaa tgaagtgtta |
| 421 | tgtcgttatc acgatgtgaa tcatccaaat catatagaag agcatattgt gcaggcgtgg |
| 481 | gaacaaatta aacatgacta tagtatagat cctagagaaa cgatttattt tacgagttat |
| 541 | gattggcatg atgaagacca tcaaacggtt caatttttaa gaagctattg cttagatcag |
| 601 | tcaacggatt atataggtat acaagatatc gtcgtggcag atgatggtat atatacgcca |
| 661 | aatggtgaga gaattcatta tttatataga ctatatccaa ttgaatattt agtatcagac |
| 721 | gctgataaaa acgggaaaag aattggactt cagtttttag atcatatagc gcaaggtaga |
| 781 | gtgaaaatca ttaatccacc agctgctttt cttatgcaaa ataaaagtgt actcgcatta |
| 841 | atttggcaac tttacgaaga ggaagttttc tttgaggaag aggagcgagg aatcattcag |
| 901 | aactacttct taccgacata ttttacaaat aaaccgttta tagaaagaaa tgaatcatat |
| 961 | gtttctaaac ctctatacgg ccgtgaaggc ggaggagtat ctatatacga aaataatgaa |
| 1021 | ctattagctg aagataaaac agagtattac tttgaacagc gaaaaatata tcagcaatac |
| 1081 | gtagaaatgc ctgactatac gattgacaca tgggacggtc cgtatactgg taaactatta |
| 1141 | attggttcac actgtattag cgggagagct gctggtttat ttttacgtgt aggtgagaaa |
| 1201 | ataacgggaa acttatcgat gtttactgga gttacaattg aaggataa |

BAS2724
Accession No. NC_005945
Bacillus anthracis str. Sterne, complete genome.

SEQ ID NO: 82

MYTEQEQKDYMKVWFSLAEEAGRNGFTWPSLLENEEWNQYMATGMYRMPIKTY
TAISKATEEIMYVLYRTYQYILNTSKDFQKLGFPAETWEIARMKHTGLFSYFTRFDFI
VNSEDIKLIEVNCDTPTGYLEPSVANEVLCRYHDVNHPNHIEEHIVQAWEQIKHDYS
IDPRETIYFTSYDWHDEDHQTVQFLRSYCLDQSTDYIGIQDIVVADDGIYTPNGERIH
YLYRLYPIEYLVSDADKNGKRIGLQFLDHIAQGRVKIINPPAAFLMQNKSVLALIWQ
LYEEEVFFEEEERGIIQNYFLPTYFTNKPFIERNESYVSKPLYGREGGGVSIYENNELL
AEDKTEYYFEQRKIYQQYVEMPDYTIDTWDGPYTGKLLIGSHCISGRAAGLFLRVG
EKITGNLSMFTGVTIEG
BAS1283
Accession No. NC_005945, REGION: 1317580 . . . 1318674
Bacillus anthracis str. Sterne, complete genome.
Bases 1 to 1095
ORIGIN

SEQ ID NO: 83

| | |
|---|---|
| 1 | atgtttacaa gtcgagtcat agatacatta caaattaagt atcccattat tcaagcaggt |
| 61 | atggcgggtg cgattacgac gccaaaactt gttgcagctg taagtaatag cggaggatta |
| 121 | ggtactcttg gagcaggcta tatgagccca gaacaaattc gtgaagcaat ttatacaata |
| 181 | agggagctaa cagataagcc cttcggtgtt aatttacttt taacgaaaga ggtacagata |
| 241 | gaagaagaga agataaactt gggaaaggga ttacttagcg gagtgaatag agaattcggt |
| 301 | atagaggaag aagagcagtt aaagcttcca aaaagttata aagaacaatt ccaagtgtta |
| 361 | ttagaagaaa aagtaccagt cgttagcttt gcgtttcaaa cgttagaaaa agaagagata |
| 421 | aatgatttga aaagaagtgg aataaaagtc atcggcacag ctactcatgt ggcagaggcg |
| 481 | aaagtgcttg ctgaattagg agtagacatt attgtcggtc aaggtagcga ggcaggaggg |
| 541 | catagaggaa cgtttatcgg gaaagaacag gacgctatga ttggtacgtt tgcattaatt |
| 601 | ccgcagctag tagcagcagt tccgcatatc ccgattgttg cagtaggtgg tgtaatgaac |
| 661 | ggacaagggc ttgttgctgc atttacactg ggggcagaag ctgttcaaat gggatcagcc |
| 721 | tttttaacga gtgaagaaag tattacgcat gatgtgtata aagaagcagt tttacatagt |
| 781 | acagatacga gcacaactgt aactcgggcg ttttccggga aatatgcacg cggtattcgt |
| 841 | aatgaattta tagagaagca tgaagggaaa gaagaagggc ttccgatgta tccggtgcaa |
| 901 | aatgtattaa cttctaaaat acgccaagaa gcagcaaaac aaaataatgg agaatatatg |
| 961 | tcactttggg cgggacaagc gtcgtcatta gcacgaatag aatcagctca gcatgtagtg |
| 1021 | gagcgagtta tggaagaagc aaataacgta atcgaacaat tacagaatgt atatagaaaa |
| 1081 | agaccacttg aataa |

BAS1283
Accession No. NC_005945
Bacillus anthracis str. Sterne, complete genome.

SEQ ID NO: 84

MFTSRVIDTLQIKYPIIQAGMAGAITTPKLVAAVSNSGGLGTLGAGYMSPEQIREAIY
TIRELTDKPFGVNLLLTKEVQIEEEKINLGKGLLSGVNREFGIEEEEQLKLPKSYKEQ
FQVLLEEKVPVVSFAFQTLEKEEINDLKRSGIKVIGTATHVAEAKVLAELGVDIIVGQ
GSEAGGHRGTFIGKEQDAMIGTFALIPQLVAAVPHIPIVAVGGVMNGQGLVAAFTL
GAEAVQMGSAFLTSEESITHDVYKEAVLHSTDTSTTVTRAFSGKYARGIRNEFIEKH
EGKEEGLPMYPVQNVLTSKIRQEAAKQNNGEYMSLWAGQASSLARIESAQHVVER
VMEEANNVIEQLQNVYRKRPLE
BAS1282
Accession No. NC_005945, REGION: complement(1316544 . . . 1317464)
Bacillus anthracis str. Sterne, complete genome.
Bases 1 to 921
ORIGIN

SEQ ID NO: 85

| | |
|---|---|
| 1 | atgatatatc caaatgtatt acaacaaggt gatacagtaa tgattattgc accgtccggc |
| 61 | ccaccaacaa ttgaaaatgt attaaaaggt gtaaaggcat tacaagaaat gggtttatct |
| 121 | gtagtaatcg ggaagagtgt ttatgagaaa tatggatatt tagctggaag ggatcaagtc |
| 181 | cggcttgatg atatacatga agcattttca aatcatgaag taaaggccgt tttctgtgca |
| 241 | cgaggtggtt acggtagcgc tcgtctcctc cctcacattc aatatgaaat cattcggaaa |
| 301 | aatccaaaaa tcttttgggg atatagcgat attacagctt tacatactgc cttttcacgt |
| 361 | tatgcaaagc ttattacttt tcatggccca atggttgaag aattagggaa aggtatagat |
| 421 | tctctttctt tatcttcttt caaccaacta tttcatccgt attcaaccat tttatctgcg |
| 481 | tcagaatgta tcgtacctag ctcttcccgt acaattacag gtcaattagt tggagggaat |
| 541 | ttagcagtgc taacgagcat aattggctca cactacgagg tacatacagc caataaactt |
| 601 | ttattacttg aagacattgg ggaagaaccg tatcgcgttg atcgtatgtt aaatcaacta |
| 661 | ctcttatctg gaaagttcaa tgaatgcagt ggtgttattt ttacaagctg tcacgactgt |
| 721 | actccttcta aaccatctca atcattgcaa acgatactat atgaatattt cgcaccgtat |
| 781 | catatacctg tcctattcgg tttaccgatt ggacatataa gcccaaacat tggaattcct |
| 841 | cttggagcta cagctacaat aagtacacat aataaaacac tctctatttc ttctggcgta |
| 901 | gccaccccgt gttcaaatta a |

BAS1282
Accession No. NC_005945
Bacillus anthracis str. Sterne, complete genome.

SEQ ID NO: 86

MIYPNVLQQGDTVMIIAPSGPPTIENVLKGVKALQEMGLSVVIGKSVYEKYGYLAG
RDQVRLDDIHEAFSNHEVKAVFCARGGYGSARLLPHIQYEIIRKNPKIFWGYSDITA
LHTAFSRYAKLITFHGPMVEELGKGIDSLSLSSFNQLFHPYSTILSASECIVPSSSRTIT
GQLVGGNLAVLTSIIGSHYEVHTANKLLLLEDIGEEPYRVDRMLNQLLLSGKFNECS
GVIFTSCHDCTPSKPSQSLQTILYEYFAPYHIPVLFGLPIGHISPNIGIPLGATATISTHN
KTLSISSGVATPCSN
BAS4563
Accession No. NC_005945, REGION: 4467874 . . . 4469040
Bacillus anthracis str. Sterne, complete genome.
Bases 1 to 1167
ORIGIN

SEQ ID NO: 87

| | |
|---|---|
| 1 | atgagtagcg cgtttattta ttcggatgac tttcggggct attcatttag ccctgatcat |
| 61 | ccctttaacc aactgcgcgt cacacttacg tatgatttat tacaaaaggg cggtttcatc |
| 121 | tctccttccc aaatcatctc accacggatg gctacagatg aagagattgc ctacattcat |
| 181 | acagaggagt acataaatgc ggtaaaacgt gctggagaag gtaagttaga aaaatcaatt |
| 241 | gcgatgacat atggactcgg aacagaagat acaccaatgt ttccaaatat gcacgaagca |
| 301 | agcgcattac tcgttggcgg tacgttaacc gctgtcgatg ctgttctttc tgggaaagta |
| 361 | aaacacgctc tcaatttagg tggtggctta catcatggct tccgtggcaa agcatctggc |
| 421 | ttttgcattt ataacgatag ttccatcgca atgaaatata ttcaaaagaa gtacggttta |
| 481 | cgcgttttat atattgatac ggatgctcat cacggtgatg gtgtacagtg gtccttttat |
| 541 | gacgatccta acgtatgcac catttcacta catgaaactg gtcgatattt attccctgga |
| 601 | actggcgctg taaacgaacg cggacaaggt aatggctata gttattcttt taacgttcca |
| 661 | ctcgatgctt ttacagaaga cgaatcgttt ttagattcct atcgaactgt tgtaaaagaa |
| 721 | gtggccgcat actttaaacc ggatattatt ttaacgcaaa atggtgctga cgcacattac |
| 781 | tacgacccac ttacacacct ttgcgcaacg atgaatattt accgcgagat accaaagctc |
| 841 | gctcgcgaaa tcgctaacga atattgcgaa ggtcgctgga ttgctgtcgg cggcggtggc |
| 901 | tatgaccact ggcgtgtcgt cccaagagct tgggcactca tttggctcga aatgaacaac |
| 961 | atccaaaaca tctcaggtta tctccctcca gaatggattg acgcttggaa aggacaagct |
| 1021 | gaaacagaac ttcctctcac atgggaagat ccaaacaaca tgtataaacc tatcccccgc |
| 1081 | aaaccagaaa ttgaagaaaa gaacgcatta actgtagcaa aatcccttga aattattcgg |
| 1141 | aataatatga aaaaatcttt gtactaa |

BAS4563
Accession No. NC_005945
Bacillus anthracis str. Sterne, complete genome.

SEQ ID NO: 88

MSSAFIYSDDFRGYSFSPDHPFNQLRVTLTYDLLQKGGFISPSQIISPRMATDEEIAYI
HTEEYINAVKRAGEGKLEKSIAMTYGLGTEDTPMFPNMHEASALLVGGTLTAVDA
VLSGKVKHALNLGGGLHHGFRGKASGFCIYNDSSIAMKYIQKKYGLRVLYIDTDAH
HGDGVQWSFYDDPNVCTISLHETGRYLFPGTGAVNERGQGNGYSYSFNVPLDAFT
EDESFLDSYRTVVKEVAAYFKPDIILTQNGADAHYYDPLTHLCATMNIYREIPKLAR
EIANEYCEGRWIAVGGGGYDHWRVVPRAWALIWLEMNNIQNISGYLPPEWIDAWK
GQAETELPLTWEDPNNMYKPIPRKPEIEEKNALTVAKSLEIIRNNMKKSLY
BAS4985
Accession No. NC_005945, REGION: complement(4859721 . . . 4861016)
Bacillus anthracis str. Sterne, complete genome.
Bases 1 to 1296
ORIGIN

SEQ ID NO: 89

| | |
|---|---|
| 1 | atgtcaacaa ttattgatgt ttatgctcgc gaagtccttg actctcgtgg taacccaact |
| 61 | gtagaagtag aagtttacac agaaagcggc gctttcggac gcgctatcgt accaagtggt |
| 121 | gcatctactg gtgagcacga agcagtagaa ttacgtgacg gtgacaaatc tcgttacctt |
| 181 | ggtaaaggtg ttatgaacgc agtaaacaac gttaatgaag caatcgctcc agaaatcgtt |
| 241 | ggtttcgacg taactgacca agctggtatc gaccgtgcta tgatcgaatt agatggcact |
| 301 | ccaaacaaag gtaaactagg cgctaacgct atccttggtg tatctatggc agtagctcac |
| 361 | gcagcagctg acttcgtagg tcttccatta taccgttacc ttggtggatt caatgcaaaa |
| 421 | caattaccaa ctccaatgat gaacatcatc aacggtggtt ctcacgctga taacaacgtt |
| 481 | gacttccaag agttcatgat cttaccagtt ggtgctccaa cattcaaaga atcaatccgt |
| 541 | atgggtgctg aagtattcca tgcacttaaa gctgtattac atgacaaagg tcttaacact |
| 601 | gcagtaggtg acgaaggtgg attcgctcca aaccttggtt ctaaccgtga agcattagaa |
| 661 | gtaatcatcg aagctatcga aaaagctggt tacaaagctg gcgagaacgt attcttagga |
| 721 | atggacgttg cttcttctga gttctacaac aaagaaactg gtaaatatga ccttgcaggt |
| 781 | gaaggccgta ctggcttaac ttctgcagaa atggttgatt tctacgaaga gctttgcaaa |
| 841 | gacttcccaa tcatctctat cgaagatggt ttagacgaaa acgactggga tggtcacaaa |
| 901 | ttattaactg agcgtatcgg tgataaagta caattagttg gtgacgattt attcgtaact |
| 961 | aacactcaaa aacttgctga aggtatcgaa aaaggtatct ctaactcaat cttaattaaa |
| 1021 | gttaaccaaa tcggtacttt aactgagact ttcgaagcta tcgaaatggc taaacgtgct |
| 1081 | ggttacacag cagttgtatc tcaccgttct ggtgaaactg aagatgctac aattgctgac |
| 1141 | atcgcagttg caactaacgc tggccaaatc aaaactggtt ctatgagccg tactgaccgt |
| 1201 | attgctaagt acaaccaatt attacgcatc gaagacgaac taggcgaaat cgctgtttac |
| 1261 | gatggtatca aatcttttta taacatcaaa cgataa |

BAS4985
Accession No. NC_005945
Bacillus anthracis str. Sterne, complete genome.

SEQ ID NO: 90

MSTIIDVYAREVLDSRGNPTVEVEVYTESGAFGRAIVPSGASTGEHEAVELRDGDKS
RYLGKGVMNAVNNVNEAIAPEIVGFDVTDQAGIDRAMIELDGTPNKGKLGANAIL
GVSMAVAHAAADFVGLPLYRYLGGFNAKQLPTPMMNIINGGSHADNNVDFQEFMI
LPVGAPTFKESIRMGAEVFHALKAVLHDKGLNTAVGDEGGFAPNLGSNREALEVII
EAIEKAGYKAGENVFLGMDVASSEFYNKETGKYDLAGEGRTGLTSAEMVDFYEEL
CKDFPIISIEDGLDENDWDGHKLLTERIGDKVQLVGDDLFVTNTQKLAEGIEKGISNS
ILIKVNQIGTLTETFEAIEMAKRAGYTAVVSHRSGETEDATIADIAVATNAGQIKTGS
MSRTDRIAKYNQLLRIEDELGEIAVYDGIKSFYNIKR
BAS0331
Accession No. NC_005945, REGION: 357064 . . . 358293
Bacillus anthracis str. Sterne, complete genome.
Bases 1 to 1230
ORIGIN

SEQ ID NO: 91

| | |
|---|---|
| 1 | atgtctaagt tcacaaagta ttttttaatg gaagctaacg atgtaattgt atatgtgaaa |
| 61 | gagaaattat gtaagtttga acatgcaaag gggttacagt gtaaagaaat aggtgatggt |
| 121 | aatttaaatt atgtattccg cgtttgggat gaacagaaga acatttctgt cattgtaaag |
| 181 | caagctgggg atacagctcg tatttcagat gagtttaagt tatcgacgaa tcgtattcgt |
| 241 | attgaatcag atgttttgca gttagaggaa gagttagcac ctggacttgt tccgaaggtg |
| 301 | tatttgtttg atagtgtgat gaattgttgc gtaatggagg acttatcgga tcacacaata |
| 361 | ttacgtacag cacttataaa tcatgaaata tttccgaggc ttgcggatga tttaacgacc |
| 421 | tttttggtaa atacgctctt attaacatcg gatgttgtaa tgaatcataa agagaagaag |
| 481 | gaacttgtga agaattatat aaatcctgag ttatgtgaga ttacagaaga cctcgtatac |
| 541 | gctgagccat ttacaaatca taataagcgt aatgagttat ttccgttaaa tgaagggtgg |
| 601 | attagagaac atatttatag tgataaagag cttcgtatag aagtagcaaa acttaagttt |
| 661 | tcttttatga cgaatgcaca ggcgcttatt cacggtgatt tgcatactgg ttctgttttt |
| 721 | gtaaaaaatg attccacaaa ggtaattgat cctgagtttg ccttttatgg accaatgggc |
| 781 | tatgacattg ggaatgtaat ggcgaattta atgtttgctt gggtgaatgc agatgcgaca |
| 841 | atgtcagctg gagccaagaa agatacgtat atggattggt tacaatcgac aatggtagag |
| 901 | gtaattgatc tatttaagaa gaagttttta gatgcttgga atattcatgt gacagagatt |
| 961 | atggcgaaag aagaaggctt taacgaaatc tatttacaat ctgtattaga ggatacagct |
| 1021 | gcagtgacag gccttgagtt aattcgtcgt attgttgggc tagcgaaagt aaaagatatt |
| 1081 | acttgtattg agaatgagga agcacgtgct agagcagaac gcatttgtct tcaagtagca |
| 1141 | aagaaattta ttttacgagc gaatcaatat aaaacaggta caagctttgt agaaacgtta |
| 1201 | aaagaacagt caatgcacta tgcgaagtaa |

BAS0331
Accession No. NC_005945
Bacillus anthracis str. Sterne, complete genome.

SEQ ID NO: 92

MSKFTKYFLMEANDVIVYVKEKLCKFEHAKGLQCKEIGDGNLNYVFRVWDEQKNI
SVIVKQAGDTARISDEFKLSTNRIRIESDVLQLEEELAPGLVPKVYLFDSVMNCCVM
EDLSDHTILRTALINHEIFPRLADDLTTFLVNTLLLTSDVVMNHKEKKELVKNYINPE
LCEITEDLVYAEPFTNHNKRNELFPLNEGWIREHIYSDKELRIEVAKLKFSFMTNAQ
ALIHGDLHTGSVFVKNDSTKVIDPEFAFYGPMGYDIGNVMANLMFAWVNADATM
SAGAKKDTYMDWLQSTMVEVIDLFKKKFLDAWNIHVTEIMAKEEGFNEIYLQSVL
EDTAAVTGLELIRRIVGLAKVKDITCIENEEARARAERICLQVAKKFILRANQYKTG
TSFVETLKEQSMHYAK
BAS4039
Accession No. NC_005945, REGION: complement(3975031 . . . 3976257)
Bacillus anthracis str. Sterne, complete genome.
Bases 1 to 1227
ORIGIN

SEQ ID NO: 93

| | |
|---|---|
| 1 | atgatgatta aagtagcgtc tattacaaaa gtagaagatg gttcgattgt aacgccaaaa |
| 61 | ggtttctcgg ccattggcac tgcaattggt ctgaaaaagg ggaaaaagga tttaggggca |
| 121 | atcgtttgtg atgtaccggc atcatgtgct gctgtttata caacaaatca aatacaagca |
| 181 | gccccgttgc aagtgacgaa ggatagtata acgactgagg ggaaactaca agctattatc |
| 241 | gttaatagtg gaaatgcaaa tgcttgtaca ggaatgaaag ggttgcaaga tgcttacgag |
| 301 | atgcgtgcat taggggcgga acattttgga ttgaaagaaa agtatgttgc agtagcttca |
| 361 | acaggtgtaa ttggtgttcc gctgccgatg gatataatcc gaaagggaat tgtaactctt |
| 421 | ataccggcga aggaagaaaa tggagctcat tctttttctg aagcaatttt aacgacggat |
| 481 | cttataacga aagaaacttg ctatgaaatg attattgatg ggaagaaagt gatgattgct |
| 541 | ggtgttgcga aaggttcagg gatgattcat ccaaatatgg caacgatgct aagttttatt |
| 601 | acgacagacg ctcgtataga gcatgacgta ttgcaaacag cattatcaca aataacgaat |
| 661 | catacattta atcaaattac agtagatgga gatacttcta cgaatgatat ggtcatcgct |
| 721 | atggcaagtg gattatcaga aacgaaacca atcgatatgg aacatgcaga ttgggaaact |
| 781 | ttcgtatttg ctttacagaa ggtatgtgaa gatttagcca aaaaaattgc acaagatggt |
| 841 | gaaggtgcta cgaagttaat agaagtaaat gtgctaggag ttcaaacaaa tgaagaggca |
| 901 | aagaaaatcg caaagcaaat agtcggttca agtcttgtga aaacagcaat acatggtgaa |
| 961 | gacccaaatt gggggcgaat tattagcagt attggacaaa gtgaagtagc aattaatccg |
| 1021 | aatacaattg acattactct tcaatctata tcggtattaa aaaatagtga gcctcaaaca |
| 1081 | ttttctgaag aagaaatgaa agagagatta caagaagatg aaatagtcat taatgtgtat |
| 1141 | ttacatttag gtaaagagac aggatcagct tggggctgtg acttaagcta tgaatatgtg |
| 1201 | aaaataaacg cttgttatcg tacataa |

BAS4039
Accession No. NC_005945
Bacillus anthracis str. Sterne, complete genome.

SEQ ID NO: 94

MMIKVASITKVEDGSIVTPKGFSAIGTAIGLKKGKKDLGAIVCDVPASCAAVYTTNQ
IQAAPLQVTKDSITTEGKLQAIIVNSGNANACTGMKGLQDAYEMRALGAEHFGLKE
KYVAVASTGVIGVPLPMDIIRKGIVTLIPAKEENGAHSFSEAILTTDLITKETCYEMII
DGKKVMIAGVAKGSGMIHPNMATMLSFITTDARIEHDVLQTALSQITNHTFNQITV
DGDTSTNDMVIAMASGLSETKPIDMEHADWETFVFALQKVCEDLAKKIAQDGEGA
TKLIEVNVLGVQTNEEAKKIAKQIVGSSLVKTAIHGEDPNWGRIISSIGQSEVAINPN
TIDITLQSISVLKNSEPQTFSEEEMKERLQEDEIVINVYLHLGKETGSAWGCDLSYEY
VKINACYRT
BAS4040
Accession No. NC_005945, REGION: complement(3976266 . . . 3977303)
Bacillus anthracis str. Sterne, complete genome.
Bases 1 to 1038
ORIGIN

SEQ ID NO: 95

| | |
|---|---|
| 1 | atgaaggtcg cgattattgg agcaactggg tatggaggta ttgagttaat tcggttatta |
| 61 | gaacaacatc catatttttc gatagcatct ctccattctt tttcacaagt tggcgagtgt |
| 121 | ataacaaatg tatatccgca ttttcaaaat gttcttgttc atacgttaca agaaattgat |
| 181 | gtggaggaaa tagagaagga agcagaaatt gtatttttag caaccccagc aggagtatca |
| 241 | gcagagttaa ctcccaaatt attagcagta ggcttaaaag taattgacct atctggagac |
| 301 | tttcgtatga aagatccttt catatatgaa cagtggtata aaagggcagc tgcaaaagaa |
| 361 | ggagtcctta gggaagctgt atatgggtta agtgaatgga aaaggtccga aattcaaaag |
| 421 | gcaaatttaa ttgcaaaccc gggatgtttt gctacagctg cattattagc gatattaccg |
| 481 | ttagttcgta gcggcataat tgaggaagac tcaattatta ttgatgcgaa atcaggagta |
| 541 | tctggagcag gcaaaacgcc aacaacgatg actcactttc ctgagttata tgataacttg |
| 601 | cgtatttata aagtaaatga gcatcaacac attcctgaga ttgagcaaat gctcgcggag |
| 661 | tggaatagag aaacgaagcc aatcacgttt agtacacatt taataccgat atcacgtggg |
| 721 | attatggtta cactgtatgc gaaagtaaag cgagaaatgg aaatagaaca acttcaacaa |
| 781 | ttatatgaag aagcgtatga acaatcggct tttattcgaa ttcgcatgca aggagagttt |
| 841 | ccaagtccga aagaagtgag aggctcaaat tattgtgata tggggatagc ttacgatgaa |
| 901 | agaacaggaa gagtgacaat tgtttctgtt atagacaata tgatgaaagg tgcggctggt |
| 961 | caagcgattc aaaatgcaaa tatagtagcg ggactagaag aaacgacagg tttacaacat |
| 1021 | atgccgcttt atctataa |

BAS4040
Accession No. NC_005945
Bacillus anthracis str. Sterne, complete genome.

SEQ ID NO: 96

MKVAIIGATGYGGIELIRLLEQHPYFSIASLHSFSQVGECITNVYPHFQNVLVHTLQEI
DVEEIEKEAEIVFLATPAGVSAELTPKLLAVGLKVIDLSGDFRMKDPFIYEQWYKRA
AAKEGVLREAVYGLSEWKRSEIQKANLIANPGCFATAALLAILPLVRSGIIEEDSIIID
AKSGVSGAGKTPTTMTHFPELYDNLRIYKVNEHQHIPEIEQMLAEWNRETKPITFST
HLIPISRGIMVTLYAKVKREMEIEQLQQLYEEAYEQSAFIRIRMQGEFPSPKEVRGSN
YCDMGIAYDERTGRVTIVSVIDNMMKGAAGQAIQNANIVAGLEETTGLQHMPLYL
BAS1676
Accession No. NC_005945, REGION: complement(1695285 . . . 1696514)
Bacillus anthracis str. Sterne, complete genome.
Bases 1 to 1230
ORIGIN

SEQ ID NO: 97

| | |
|---|---|
| 1 | atggaaacga ttgtacaaaa atttggtggc acttctgtcg gaagcgttga acgcattcaa |
| 61 | catgtagcaa atttaattat tgaagaatat gaacgaggac atagtatcgt ctctgtcgtt |
| 121 | tcagcaatgg ggaaaagtac agacgaactt gtagcacttg ctaacgcgat tacagaaaat |
| 181 | ccgagtaaac gcgaaatgga tatgcttcta tcaacaggag agcaagtcac tatttcatta |
| 241 | ttaacgatgg cattacaagc aaaaggttat aacgcaattt cattaacagg atggcaagct |
| 301 | ggtattacga cagaatctgt acatagtagt gcacggatta ctgaaattaa tacggatcga |
| 361 | attcagtctt atcttactaa aggcacgatt gttattgtag ctggtttcca aggagttagt |
| 421 | gaagaccttg aaattacaac gcttggacgt ggtggttctg atacaactgc tgttgcatta |
| 481 | gctgcggcac tgaacgcaaa aaaatgtgat atttacacag atgtgaccgg cgtatatacg |
| 541 | acggatccac gagttgtaaa agatgcttat aaattagatg aaatttctta tgacgaaatg |
| 601 | ttagaacttg caaatctcgg tgctggtgta ttacacccgc gtgctgttga gtttgctaaa |
| 661 | aatcataatg tcattttaga agttcgctca agtatggaac aagaaaatgg aacaattgta |
| 721 | aaaggagaat gtaacatgga acaacaatca atcgttaaag gtattgcatt tgaagataat |
| 781 | attacacgca tcacagtgaa aggattggaa caaggatcgc tttcaactgt tttctctaca |
| 841 | ttagcagcgg cacatattaa tgtagacatc atcattcaaa gtattacaaa tgaaggaact |
| 901 | gttcatctct ccttctccat ccattctaat gatttaagag aaacgttagc agtgttggaa |
| 961 | caaaatcaag aggcacttca ctatgaatca gtagaatatg aaaatcattt agcaaaagta |
| 1021 | tcaattgtag gatctggtat ggtctctaat ccaggtgtcg ctgcgaatat gttcactact |
| 1081 | ttaaaagaag aagatattca tattaaaatg gtaagtacat cagaaattaa agtgtctgtc |
| 1141 | gttattgacc gccttcattt agtaacaggt gtcgaggcgc tgcatcaatc atttatggcg |
| 1201 | aaaattgagc ctttagtgca gatgagttaa |

BAS1676
Accession No. NC_005945
Bacillus anthracis str. Sterne, complete genome.

SEQ ID NO: 98

METIVQKFGGTSVGSVERIQHVANLIIEEYERGHSIVSVVSAMGKSTDELVALANAI
TENPSKREMDMLLSTGEQVTISLLTMALQAKGYNAISLTGWQAGITTESVHSSARIT
EINTDRIQSYLTKGTIVIVAGFQGVSEDLEITTLGRGGSDTTAVALAAALNAKKCDIY
TDVTGVYTTDPRVVKDAYKLDEISYDEMLELANLGAGVLHPRAVEFAKNHNVILE
VRSSMEQENGTIVKGECNMEQQSIVKGIAFEDNITRITVKGLEQGSLSTVFSTLAAA
HINVDIIIQSITNEGTVHLSFSIHSNDLRETLAVLEQNQEALHYESVEYENHLAKVSIV
GSGMVSNPGVAANMFTTLKEEDIHIKMVSTSEIKVSVVIDRLHLVTGVEALHQSFM
AKIEPLVQMS
BAS3739
Accession No. NC_005945, REGION: complement(3707491 . . . 3708777)
Bacillus anthracis str. Sterne, complete genome.
Bases 1 to 1287
ORIGIN

SEQ ID NO: 99

| | |
|---|---|
| 1 | atgaattatt tgtttaaaaa tggtcgttat atgaatgaag aaggaaaaat cgtagcaacg |
| 61 | gatcttctag tacaagacgg taaaatcgct aaagtagcag aaaatattac ggcagataat |
| 121 | gctgaagtga tcgatgtgaa cggaaagtta atcgcacctg gattagtaga tgtacacgta |
| 181 | caccttcgtg aaccaggtgg tgaacataaa gaaacaattg aaacaggtac attagcagcg |
| 241 | gcaaaaggtg gattcactac aatttgcgca atgccaaata cacgcccagt accagattgc |
| 301 | agagaacata tggaagactt gcaaaatcgt attaaagaaa aagcacatgt taacgtacta |
| 361 | ccatatggag caattacagt acgtcaagcc ggttctgaaa tgacagattt cgaaacatta |
| 421 | aaagagcttg gagcatttgc tttcactgat gacggtgtag gcgtacaaga tgctagcatg |
| 481 | atgttagctg ctatgaagcg tgcagcgaaa ttaaatatgg cagtagttgc gcactgtgaa |
| 541 | gagaatactc ttattaataa aggttgtgta catgaaggga agttttctga gaaacacgga |
| 601 | ttaaacggta tcccatcagt atgtgaatct gtacatattg caagggatat actgcttgct |
| 661 | gaagcagcag attgtcacta tcacgtatgt cacgtaagta cgaaaggctc tgtacgcgta |
| 721 | attcgtgatg caaagcgcgc tggaattaaa gtaacagcag aggtaacgcc tcatcactta |
| 781 | gtgttatgtg aagatgatat cccatcagct gatcctaatt ttaaaatgaa cccaccgctt |
| 841 | cgtggaaaag aagaccacga agcattaatt gaaggtttat tagatggaac aatcgatatg |
| 901 | atcgcaactg accatgcacc gcatacagca gaagagaaag cgcaaggaat tgaaagagca |
| 961 | ccattcggga ttactggttt tgaaactgca ttcccacttc tatacacaaa ccttgtgaaa |
| 1021 | aaaggaatta ttacactaga gcagttaatt caattcttaa cagaaaagcc agctgataca |
| 1081 | ttcggcttag aagcaggtcg cctgaaagaa ggtagaacag ctgatattac aatcattgat |
| 1141 | ttagaacaag aagaagagat tgacccaaca acattcttat caaaaggaaa aaatacacca |
| 1201 | ttcgcaggtt ggaaatgcca aggatggccg gtaatgacaa tcgttggtgg taagatcgca |
| 1261 | tggcaaaagg agagtgcatt agtatga |

BAS3739
Accession No. NC_005945
Bacillus anthracis str. Sterne, complete genome.

SEQ ID NO: 100

MNYLFKNGRYMNEEGKIVATDLLVQDGKIAKVAENITADNAEVIDVNGKLIAPGL
VDVHVHLREPGGEHKETIETGTLAAAKGGFTTICAMPNTRPVPDCREHMEDLQNRI
KEKAHVNVLPYGAITVRQAGSEMTDFETLKELGAFAFTDDGVGVQDASMMLAAM
KRAAKLNMAVVAHCEENTLINKGCVHEGKFSEKHGLNGIPSVCESVHIARDILLAE
AADCHYHVCHVSTKGSVRVIRDAKRAGIKVTAEVTPHHLVLCEDDIPSADPNFKM
NPPLRGKEDHEALIEGLLDGTIDMIATDHAPHTAEEKAQGIERAPFGITGFETAFPLL
YTNLVKKGIITLEQLIQFLTEKPADTFGLEAGRLKEGRTADITIIDLEQEEEIDPTTFLS
KGKNTPFAGWKCQGWPVMTIVGGKIAWQKESALV
BAS4303
Accession No. NC_005945, REGION: complement(4214707 . . . 4215219)
Bacillus anthracis str. Sterne, complete genome.
Bases 1 to 513
ORIGIN

SEQ ID NO: 101

| | |
|---|---|
| 1 | atggatttca agcaacatat cgcaattgta ccggactatc caaaagaagg tatcgtgttt |
| 61 | aaagacatta caccgttaat gaacgacggt aaagcataca aagcagcaac agatgcaatc |
| 121 | gttgagtatg caaaagagag agacatcgac cttgtagtag gtccagaagc tcgtggtttt |
| 181 | attattggtt gcccagtttc ttacgcatta gaagtaggat ttgcgccagt tcgtaaatta |
| 241 | ggaaaattac cacgtgaagt aattacagtt gactacggta aagaatatgg taaagatgtt |
| 301 | ttaacaatcc ataaagatgc aattaaacca ggccaacgcg tattaattac agatgatcta |
| 361 | ttagctacag gtggaacaat cgaagcgaca attaagctag ttgaagagct aggcggagtt |
| 421 | gtagcaggaa ttgcattctt agtagaactt acttacttag atggtcgtaa aatgttagat |
| 481 | ggttacgatg tattagtatt agaaaaatac taa |

BAS4303
Accession No. NC_005945
Bacillus anthracis str. Sterne, complete genome.

SEQ ID NO: 102

MDFKQHIAIVPDYPKEGIVFKDITPLMNDGKAYKAATDAIVEYAKERDIDLVVGPE
ARGFIIGCPVSYALEVGFAPVRKLGKLPREVITVDYGKEYGKDVLTIHKDAIKPGQR
VLITDDLLATGGTIEATIKLVEELGGVVAGIAFLVELTYLDGRKMLDGYDVLVLEKY
BAS5179
Accession No. NC_005945, REGION: complement(5059109 . . . 5059693)
Bacillus anthracis str. Sterne, complete genome.
Bases 1 to 585
ORIGIN

SEQ ID NO: 103

| | |
|---|---|
| 1 | atgtacttaa taaatcagaa cggttggatt gaagtgattt gcggtagtat gttttctggt |
| 61 | aaatcagaag agcttatccg ccgtgttcgt cgtacgcaat ttgcgaaaca acatgcaatt |
| 121 | gtatttaaac catgtattga taatcgctat agtgaagaag acgttgtatc acataacgga |
| 181 | ttaaaggtaa aagcagttcc tgtttcagct tcaaaagata tatttaaaca tatcactgaa |
| 241 | gaaatggatg taattgcaat tgatgaagtg caattctttg atggggacat tgtggaagtg |
| 301 | gtgcaagtat tggcaaatcg tggctatcgt gtcattgtag ctggtttaga ccaagatttc |
| 361 | cgtggtctac catttggaca agttcctcag ctgatggcga ttgctgaaca tgtaacaaaa |
| 421 | ctacaagctg tatgttctgc atgtggatct ccggcaagtc gtacacaacg attaattgat |
| 481 | ggagaaccag cggcatttga tgatccaatt attttagttg gtgcttcaga gtcgtatgaa |
| 541 | ccacgctgtc gtcattgtca tgcagttcct acaaaacaaa gataa |

BAS5179
Accession No. NC_005945
Bacillus anthracis str. Sterne, complete genome.

SEQ ID NO: 104

MYLINQNGWIEVICGSMFSGKSEELIRRVRRTQFAKQHAIVFKPCIDNRYSEEDVVS
HNGLKVKAVPVSASKDIFKHITEEMDVIAIDEVQFFDGDIVEVVQVLANRGYRVIVA
GLDQDFRGLPFGQVPQLMAIAEHVTKLQAVCSACGSPASRTQRLIDGEPAAFDDPII
LVGASESYEPRCRHCHAVPTKQR
BAS5180
Accession No. NC_005945, REGION: complement(5059802 . . . 5060047)
Bacillus anthracis str. Sterne, complete genome.
Bases 1 to 246
ORIGIN

SEQ ID NO: 105

| | |
|---|---|
| 1 | atgaaagcag gaattcaccc agattacaag aaagttgtat tcatggacac aaacacaggc |
| 61 | ttcaaattct taagcggatc tactaaagga tctaacgaaa ctgttgagtg ggaagatggt |
| 121 | aacacttatc cattactaaa agttgagatc agttctgatt ctcacccatt ctacactgga |
| 181 | cgtcagaagt ttgctactgc agacggacgc gttgaccgct tcaataagaa atacggtctt |
| 241 | aagtaa |

BAS5180
Accession No. NC_005945
Bacillus anthracis str. Sterne, complete genome.

SEQ ID NO: 106

MKAGIHPDYKKVVFMDTNTGFKFLSGSTKGSNETVEWEDGNTYPLLKVEISSDSHP
FYTGRQKFATADGRVDRFNKKYGLK
BAS0286
Accession No. NC_005945, REGION: 307243 . . . 308514
Bacillus anthracis str. Sterne, complete genome.
Bases 1 to 1272
ORIGIN

SEQ ID NO: 107

| | |
|---|---|
| 1 | atgaatgttt tagtaattgg ccgcggtggg cgtgagcatg ctttagcttg gaagtttgca |
| 61 | caatctgaaa aagtagaaaa ggtatatgta gcaccaggta atgaaggtat gcgtgatgtt |
| 121 | gcaacaccaa ttgatattga tgaaaatgat tttgatgcat tagttttatt tgcgaaagag |
| 181 | aaccatgtgg aattaacttt cgttggacca gaaattccac ttatgaatgg aattgttgat |
| 241 | cgttttaaag aagagggact tcgcgtattt ggtccgaata aagcagctgc tgttattgaa |
| 301 | ggtagtaaag cttttacaaa agagcttatg aaaaaatata atattccaac tgcag cgtac |
| 361 | gaaactttta cagattatga agaagcagta cagtacattg aaaaagttgg tgcaccaatt |
| 421 | gttattaaag cggatggtct agctgctggt aaaggtgtaa cagtagcgat gacgcttgaa |
| 481 | gaggcattac aagctgcgaa agaaatgctg caagatgtaa agttcgggga agcgagtaag |
| 541 | aaagttgtta ttgaagagtt tttagatgga caagaatttt cattaatggc atttgtgaat |
| 601 | ggaacaactg tacatccgat ggtaattgcg caagatcata aacgagcttt tgatggtgat |
| 661 | aaaggtccaa atactggcgg aatgggtgca tattctccag taccacaaat ttcggagtca |
| 721 | gcagttcaag aggcgattga aacggtgtta tatccaactg ctaaagcaat gatccaagaa |
| 781 | aatcgatcgt ttacaggaat tttatatgcg ggacttattt taacaaatga tggtccaaag |
| 841 | gtaattgaat ttaatgcacg ttttggcgat cctgaaactg aagttgtatt accccgttta |
| 901 | gaaaatgatt tagtggatgt atgtaacgct gtattagatg aaagtgagtt aacgttacaa |
| 961 | tggtcagatg aagccgtaat tggtgttgta cttgctgcga aaggatatcc ggaagcatat |
| 1021 | aaaaaaggtg acattattaa agggctagat gcattgcaag atgtgattgt tttccatgca |
| 1081 | ggtacagcaa tgaaacgtgg tgactttgta acgaacggtg gccgtgtatt atttgttgct |
| 1141 | tgtaaggcga atagtttaca agaagcgaaa gataaggtgt ataaagaaat cggtaaaatt |
| 1201 | gagagtgatg ttctatttta ccgaagtgat ataggatatc gtgcaattgg gcatgagatg |
| 1261 | acgagaagtt aa |

BAS0286
Accession No. NC_005945
Bacillus anthracis str. Sterne, complete genome.

SEQ ID NO: 108

MNVLVIGRGGREHALAWKFAQSEKVEKVYVAPGNEGMRDVATPIDIDENDFDALV
LFAKENHVELTFVGPEIPLMNGIVDRFKEEGLRVFGPNKAAAVIEGSKAFTKELMK
KYNIPTAAYETFTDYEEAVQYIEKVGAPIVIKADGLAAGKGVTVAMTLEEALQAAK
EMLQDVKFGEASKKVVIEEFLDGQEFSLMAFVNGTTVHPMVIAQDHKRAFDGDKG
PNTGGMGAYSPVPQISESAVQEAIETVLYPTAKAMIQENRSFTGILYAGLILTNDGPK
VIEFNARFGDPETEVVLPRLENDLVDVCNAVLDESELTLQWSDEAVIGVVLAAKGY
PEAYKKGDIIKGLDALQDVIVFHAGTAMKRGDFVTNGGRVLFVACKANSLQEAKD
KVYKEIGKIESDVLFYRSDIGYRAIGHEMTRS
BAS1986
Accession No. NC_005945, REGION: 1992760 . . . 1993773
Bacillus anthracis str. Sterne, complete genome.
Bases 1 to 1014
ORIGIN

SEQ ID NO: 109

| | |
|---|---|
| 1 | atgcaagagc gatattcaag acaagtattg ttttctggaa taggtgaaat gggacaaagg |
| 61 | aaaataaggg aaaagcatgt gctcttaatt ggtgcggggg cgctaggagc tgcaaatgca |
| 121 | gaagcgctcg ttcggatggg aattgggaaa ttgacaattg ccgatcgtga ttatgtcgaa |
| 181 | tggagtaatt tacaacggca acagttatat acagaagaag atgcaaaaca atgtaaacca |
| 241 | aaagcaattg cagcggcaga acatgtaaga aagattaatt ctgaggtgga aattgtacca |
| 301 | gttgtaacgg atgtcacaat gaaagaaatg gaagagttaa cgaaagaagt ggatctcata |
| 361 | ttagatgcga ctgacaactt tgatacgcgt ctacttataa atgatatttc acaaaaagaa |
| 421 | aatatacctt ggatatacgg tggatgcatt ggaagttacg gtgtaacgta cacaattctt |
| 481 | ccaggaaaaa caccatgttt tcgctgctta atggatcatc cgatgggcgg tgcaacatgt |
| 541 | gatacagcgg gaattattca gccagctgta cagatggttg ttgctcacca agtgacagaa |
| 601 | gcaatgaaaa tattggtgga tgacttcgag acgctacgag gaacaatgtt atcatttgat |
| 661 | atttggaaca atcaatttct ctcattaaaa gtaaataaac agaaaaaaag tacatgtcca |
| 721 | tcttgcggaa atacacgtac gtacccaagt ttaacatttg aatcacaggt gaaaacggag |
| 781 | gtgctatgcg gacggaatac agttcaaatc cgtccaggaa taaagagagt tctaaattta |
| 841 | aacgaaatta aaaaaagatt acaaaaaatt gtacatgtcc aaaaaacgcc gtacttacta |
| 901 | tcatttctaa ttgatgaata tcgtttcgtt ttatttacag acggtagggc atttgttcat |
| 961 | gggacaaatg atgtgaaaat aggaaaacga ttgtacgcaa aatatatagg atga |

BAS1986
Accession No. NC_005945
Bacillus anthracis str. Sterne, complete genome.

SEQ ID NO: 110

MQERYSRQVLFSGIGEMGQRKIREKHVLLIGAGALGAANAEALVRMGIGKLTIADR
DYVEWSNLQRQQLYTEEDAKQCKPKAIAAAEHVRKINSEVEIVPVVTDVTMKEME
ELTKEVDLILDATDNFDTRLLINDISQKENIPWIYGGCIGSYGVTYTILPGKTPCFRCL
MDHPMGGATCDTAGIIQPAVQMVVAHQVTEAMKILVDDFETLRGTMLSFDIWNN
QFLSLKVNKQKKSTCPSCGNTRTYPSLTFESQVKTEVLCGRNTVQIRPGIKRVLNLN
EIKKRLQKIVHVQKTPYLLSFLIDEYRFVLFTDGRAFVHGTNDVKIGKRLYAKYIG
BAS5244
Accession No. NC_005945, REGION: complement(5128504 . . . 5128788)
Bacillus anthracis str. Sterne, complete genome.
Bases 1 to 285
ORIGIN

SEQ ID NO: 111

| | |
|---|---|
| 1 | atgcaaatat caagcgataa gattttaaat aaaatggcga atgaaattgc aaaggcaaaa |
| 61 | agtagtgaag gacaaaaatc aaaagagcat ttattagttg tacgtgcttt atgcgattta |
| 121 | ttattagatg agcaagttga atcttctacg tatagagagc cgcaaattca atcacaaata |
| 181 | attggatcac agccagtaac gatgcaacca atcgcaccgg tttctggaga accagtttat |
| 241 | ataaaagaaa gtgatgcaaa cggtaattct ttatttgatt tttag |

BAS5244
Accession No. NC_005945
Bacillus anthracis str. Sterne, complete genome.

SEQ ID NO: 112

MQISSDKILNKMANEIAKAKSSEGQKSKEHLLVVRALCDLLLDEQVESSTYREPQIQ
SQIIGSQPVTMQPIAPVSGEPVYIKESDANGNSLFDF
BAS0698
Accession No. NC_005945, REGION: 757467 . . . 758243
Bacillus anthracis str. Sterne, complete genome.
Bases 1 to 777
ORIGIN

SEQ ID NO: 113

| | |
|---|---|
| 1 | ttgattatgt taaacattgg accattttca tttcattcta gacttttatt aggaacagga |
| 61 | aaattccctg attttgatgt acagcaaaag gcaattgatg tatctgaggc tgaggttcta |
| 121 | accttcgcag tacgtcgtat ggatatattt gatgcaaagc agcctaattt attagagaaa |
| 181 | cttgatgtaa aaaaatataa gttattaccg aatacagctg gagcaaaaaa tgctgaagaa |
| 241 | gctgttcgta ttgcaaaatt agcaaaagct tcagggcttt gcgacatgat taaagtagaa |
| 301 | gttataggtg atgatagaac gttattacct gatccggtag aaacattgag agcatctgaa |
| 361 | atgttattag aggaaggatt tatcgtactt ccatatacat ctgatgatgt tgtattagca |
| 421 | cgtaaattac aagaactagg tgtgcatgca attatgccag gagcatcacc aatcggttca |
| 481 | gggcttggta ttgtaaatcc attaaattta agttttatta ttgaacaagc gacagtgcca |
| 541 | gttatcgttg acgctggtat cggtagccca gctgatgcgg catttgcaat ggaattagga |
| 601 | gcggatggtg tgttattaaa tacggctgtg tcaggagcaa aagatcctat taagatggca |
| 661 | caagcaatga aattaagtat tgaggcagga cgtttaggct ttgaagcagg tcgtattgca |
| 721 | cgtaaacgtt gtgcaacagc aagtagtcct ttagaaggaa tgagcgtagt tgaataa |

BAS0698
Accession No. NC_005945
Bacillus anthracis str. Sterne, complete genome.

SEQ ID NO: 114

MIMLNIGPFSFHSRLLLGTGKFPDFDVQQKAIDVSEAEVLTFAVRRMDIFDAKQPNL
LEKLDVKKYKLLPNTAGAKNAEEAVRIAKLAKASGLCDMIKVEVIGDDRTLLPDPV
ETLRASEMLLEEGFIVLPYTSDDVVLARKLQELGVHAIMPGASPIGSGLGIVNPLNLS
FIIEQATVPVIVDAGIGSPADAAFAMELGADGVLLNTAVSGAKDPIKMAQAMKLSIE
AGRLGFEAGRIARKRCATASSPLEGMSVVE
BAS0697
Accession No. NC_005945, REGION: 757267 . . . 757470
Bacillus anthracis str. Sterne, complete genome.
Bases 1 to 204
ORIGIN

SEQ ID NO: 115

| | |
|---|---|
| 1 | ttgaatttga aaattaatgg taatcaaatt gaagtgccag agagtgtaaa aacagtagcc |
| 61 | gagctactta cacatttaga gttagataac agaattgttg tagtagagcg taataaagat |
| 121 | attttacaaa aagatgatca tacagataca tctgtttttg atggagacca aattgagatt |
| 181 | gtaactttcg taggaggcgg ttga |

BAS0697
Accession No. NC_005945
Bacillus anthracis str. Sterne, complete genome.

SEQ ID NO: 116

MNLKINGNQIEVPESVKTVAELLTHLELDNRIVVVERNKDILQKDDHTDTSVFDGD
QIEIVTFVGGG
BAS1423
Accession No. NC_005945, REGION: 1449132 . . . 1449845
Bacillus anthracis str. Sterne, complete genome.
Bases 1 to 714
ORIGIN

SEQ ID NO: 117

| | |
|---|---|
| 1 | atgcaacaat caaaagaaga aagagtacat gatgtatttg agaaaatctc tgataaatac |
| 61 | gatgtgatga attctgtaat tagttttcaa agacataaag cgtggcgcaa agagacgatg |
| 121 | cgcattatgg atgtaaaacc aggtagtaaa gcgcttgatg tatgttgcgg gacagcggac |
| 181 | tggacaattg cgctagctgg agcggtgggt gaacagggca aggttgttgg tttagacttc |
| 241 | agtgaaaata tgttatctgt tggtaagcaa aaggtagagg cgttacaatt aaaacaagta |
| 301 | gaacttctac acgggaatgc aatggaactt ccatttgaag ataacacgtt tgattatgta |
| 361 | acgattggat ttggtttacg taatgtaccg gattatatgc acgtattaaa agaaatgacg |
| 421 | cgtgtagtaa aaccaggtgg aaaagtaatt tgtttagaaa catctcaacc aacaatgatt |
| 481 | ggttttcgac aaggttatat tttatatttt aaatatatca tgccgttatt tggaaagtta |
| 541 | tttgcgaaaa gttataaaga atattcatgg ctacaagaat ctgctagtac attcccaggt |
| 601 | atgaaagaat tggctaatat gttcgaaaaa gctggacttg aacgtgtgca agtgaagcca |
| 661 | tttacttttg gggtagcagc gatgcattta ggcatgaaac cagaatcaaa atag |

BAS1423
Accession No. NC_005945
Bacillus anthracis str. Sterne, complete genome.

SEQ ID NO: 118

MQQSKEERVHDVFEKISDKYDVMNSVISFQRHKAWRKETMRIMDVKPGSKALDV
CCGTADWTIALAGAVGEQGKVVGLDFSENMLSVGKQKVEALQLKQVELLHGNAM
ELPFEDNTFDYVTIGFGLRNVPDYMHVLKEMTRVVKPGGKVICLETSQPTMIGFRQ
GYILYFKYIMPLFGKLFAKSYKEYSWLQESASTFPGMKELANMFEKAGLERVQVKP
FTFGVAAMHLGMKPESK
BAS4052
Accession No. NC_005945, REGION: complement(3986067 . . . 3987185)
Bacillus anthracis str. Sterne, complete genome.
Bases 1 to 1119
ORIGIN

SEQ ID NO: 119

| | |
|---|---|
| 1 | atgattaatc aagaacgttt agtaaatgaa ttcatggaat tagtacaagt agattctgaa |
| 61 | acgaaatttg aagcagaaat ttgcaaagta ttaacaaaga aatttacaga tttaggtgta |
| 121 | gaagtatttg aagatgacac aatggctgtt actgggcatg gtgcaggtaa cttaatttgt |
| 181 | acattaccag caacaaaaga tggtgttgat acaatttact ttacttctca tatggataca |
| 241 | gtagttcctg gtaatggaat taagccttct attaaagatg gatatatcgt atcagatggt |
| 301 | actacgattt taggtgcgga tgataaagcg ggattagcat caatgtttga agcaatccgt |
| 361 | gttttaaaag agaaaaatat ccctcacggc acaattgaat ttattattac agttggagaa |
| 421 | gaatctggtc ttgttggtgc aaaagcatta gatcgtgagc gcattacagc gaaatatggt |
| 481 | tacgcgttag atagcgatgg gaaagttggc gaaatcgttg ttgcagctcc aacacaagcg |
| 541 | aaagtgaacg cgattattcg cgggaaaaca gctcatgcag gtgtagcacc ggaaaaaggc |
| 601 | gtatctgcaa ttacgatcgc agcgaaagca attgcgaaga tgccacttgg tcgtattgat |
| 661 | tctgaaacaa ctgcaaatat tggacgtttt gaaggtggta cacaaacgaa tatcgtttgc |
| 721 | gatcatgtac aaatctttgc agaagcgcgt tctttaatca atgaaaaaat ggaagtacaa |
| 781 | gttgcgaaaa tgaaagaagc atttgaaaca actgcaaaag aaatgggcgg ccaagcagat |
| 841 | gttgaagtaa aggttatgta cccaggattt aaatttgctg atggggatca cgttgtagaa |
| 901 | gttgcaaaac gcgcagctga aaaaattggt cgtacacctt ctcttcacca aagtggtggc |
| 961 | ggaagtgatg caaacgtaat tgctggacac ggaattccaa cagttaactt agcagttggt |
| 1021 | tatgaagaaa ttcatacaac aaacgaaaag attcctgttg aagaattagc gaaaacagca |
| 1081 | gaattagttg ttgcaatcat agaggaagta gcgaaataa |

BAS4052
Accession No. NC_005945
Bacillus anthracis str. Sterne, complete genome.

SEQ ID NO: 120

MINQERLVNEFMELVQVDSETKFEAEICKVLTKKFTDLGVEVFEDDTMAVTGHGA
GNLICTLPATKDGVDTIYFTSHMDTVVPGNGIKPSIKDGYIVSDGTTILGADDKAGL
ASMFEAIRVLKEKNIPHGTIEFIITVGEESGLVGAKALDRERITAKYGYALDSDGKVG
EIVVAAPTQAKVNAIIRGKTAHAGVAPEKGVSAITIAAKAIAKMPLGRIDSETTANIG
RFEGGTQTNIVCDHVQIFAEARSLINEKMEVQVAKMKEAFETTAKEMGGQADVEV
KVMYPGFKFADGDHVVEVAKRAAEKIGRTPSLHQSGGGSDANVIAGHGIPTVNLA
VGYEEIHTTNEKIPVEELAKTAELVVAIIEEVAK
BAS5208
Accession No. NC_005945, REGION: 5097244 . . . 5098644
Bacillus anthracis str. Sterne, complete genome.
Bases 1 to 1401
ORIGIN

SEQ ID NO: 121

| | |
|---|---|
| 1 | atgaaaaaat ctttgaaaca aaaaatagta agctccttgc ttgctgtatc actcgctgtt |
| 61 | agcttagctc cgattggaca agctaacgct gattccacgt cagaaatcaa gcagacttca |
| 121 | tctatcacaa aacaagttga tgcaagccgc gctatcgaac acatccgttt cttatccgaa |
| 181 | acaattggtc ctcgacctgg tgggacaaaa tcagaagaat gggcttctcg ctacgttggt |
| 241 | atgcagctta aatcaatggg ctacgaagta gaatatcaac catttcaagt gccggatcaa |
| 301 | tacgttggat ttattgaatc accattatcc acaaagcgta attggcaaac tggtgctgcc |
| 361 | cctaatgcac taatttctac agaatctgtt acagctcctc ttatctttgt tcaaggtggg |
| 421 | acaaaattag aggatatccc aaatgaagta aatggaaaaa ttgttctatt cgaaagagga |
| 481 | acaacagtag ctgactataa taaacaagtt gaaaatgctg ttagcaaagg agcaaaaggt |
| 541 | gttcttttat acagtttaat tggtggacgt ggaaactacg gacaaacttt caatccccgc |
| 601 | ctaacgaaaa agcaatctat ccctgtcttt ggtcttgctt atgcgcaagg aaatgcattt |
| 661 | aaagaagaaa tcgctaaaaa aggaacaaca attctttccc taaaagcgag acatgaatct |
| 721 | aatttaacat cattaaacgt catcgctaaa aagaaaccaa aaaacagtac aggtaatgaa |
| 781 | aaagctgtcg ttgtaagctc acactacgat agtgtcgttg gagcacctgg agcaaatgat |
| 841 | aatgcttctg gtacaggatt agtattagaa ttagctcgtg cttttcaaaa tgtagaaact |
| 901 | gataaagaaa ttcgttttat tgcttttggt tctgaagaga ctggcttact tggctccgat |
| 961 | tattacgtta atagcttatc cccaaaagaa cgcgatcgaa ttttaggtgt ctttaacgca |
| 1021 | gacatggtcg caacaaatta cgataaagca aagaatttat atgctatgat gcctaacggt |
| 1081 | tctccaaacc ttgtaacaga cgcagcctta caagcaggta aacaattaaa taatgacctc |
| 1141 | gttctgcaag ggaaatttgg ctctagtgat cacgtaccgt ttgctgaagt tggtattcct |
| 1201 | gcggctctat ttatttggat gggtgtcgat agttggaatc cattaatcta ccatatcgaa |
| 1261 | aaggtatatc acacacctca agataacgta tttgagaata tttcacctga acgtatgaaa |
| 1321 | atggcactag aagtaatcgg aactggtgtt tataacactc ttcaacaatc tgttacgcaa |
| 1381 | acagaacaga aagctgctta a |

BAS5208
Accession No. NC_005945
Bacillus anthracis str. Sterne, complete genome.

SEQ ID NO: 122

MKKSLKQKIVSSLLAVSLAVSLAPIGQANADSTSEIKQTSSITKQVDASRAIEHIRFLS
ETIGPRPGGTKSEEWASRYVGMQLKSMGYEVEYQPFQVPDQYVGFIESPLSTKRNW
QTGAAPNALISTESVTAPLIFVQGGTKLEDIPNEVNGKIVLFERGTTVADYNKQVEN
AVSKGAKGVLLYSLIGGRGNYGQTFNPRLTKKQSIPVFGLAYAQGNAFKEEIAKKG
TTILSLKARHESNLTSLNVIAKKKPKNSTGNEKAVVVSSHYDSVVGAPGANDNASG
TGLVLELARAFQNVETDKEIRFIAFGSEETGLLGSDYYVNSLSPKERDRILGVFNAD
MVATNYDKAKNLYAMMPNGSPNLVTDAALQAGKQLNNDLVLQGKFGSSDHVPF
AEVGIPAALFIWMGVDSWNPLIYHIEKVYHTPQDNVFENISPERMKMALEVIGTGV
YNTLQQSVTQTEQKAA
BAS2981
Accession No. NC_005945, REGION: complement(2952525 . . . 2953730)
Bacillus anthracis str. Sterne, complete genome.
Bases 1 to 1206
ORIGIN

SEQ ID NO: 123

| | |
|---|---|
| 1 | atgggagcga caggagtagc gtcacaaaga aaaacaattg aagagagtat cgaaagaaat |
| 61 | aaggaaaagt acatagaaac aagtcatgat attcatgcga atccggagat tggtaatcaa |
| 121 | gaattttacg catctagaac gttaagttta ttactaggta gtgcaggatt tcagttgcag |
| 181 | cacaatatag ctggacacga aacaggattt atcgcgcgaa aaagttcagg aaaacaagga |
| 241 | ccagcaatcg catttttagc tgagtatgac gctttaccag gactcggtca tgcgtgtggt |
| 301 | cacaatttaa tcggcacaat tagcgttgca gcagcgattg cattatcaga aacactcgaa |
| 361 | gaaattggtg gagaagttgt cgtattcgga acaccagcag aagaaggcgg gccaaatggt |
| 421 | agcgcaaaat cgagttatgt aaaagcaggt ttatttaaaa atattgatgc ggcgcttatg |
| 481 | attcatccga gcggaaaaac agcgacaacg agcccttcac tagcagtcga tccacttgat |
| 541 | tttcattttt acggaaaaac agctcacgcg gcagcgtcac ctgaagaagg aattaatgca |
| 601 | ttagatgcgg tgattcagct gtacaacagc attaacgcac ttcgccaaca acttccgtca |
| 661 | gacgtgaaaa ttcatggcgt tattacagaa ggcggaaaag cacctaacat tattcctgac |
| 721 | tacgccgcag caagattctt catccgtgca gcaacgcgaa aaagatgtgc agaagtaaca |
| 781 | gaaaaagtaa aaaatattgc acagggagca gcgttagcaa cagacacaaa agtaaaaatc |
| 841 | catcaattcc aaaatgaaat cgatgaactg ctcgtaacaa aaacatataa cgacgtcgta |
| 901 | gctgaagaac tagaattact cggggaagac gtaaatcgta aagaaagatt tggtattggt |
| 961 | tcaaccgatg caggaaacgt tagccaagtt gtaccgacaa tccacccgta cattaaaatc |
| 1021 | ggcccagatg atttaattgc acatacgaat gaatttagag aagcagcacg ttcagaatta |
| 1081 | ggagacaaag ctctaattac atcagcaaaa gcactagcaa ataccgcgta tcgattaatt |
| 1141 | acagaagaag ggttgttaga gaaggtgaag gaagagttta gagaggcgca gagaaatcag |
| 1201 | gggtag |

BAS2981
Accession No. NC_005945
Bacillus anthracis str. Sterne, complete genome.

SEQ ID NO: 124

MGATGVASQRKTIEESIERNKEKYIETSHDIHANPEIGNQEFYASRTLSLLLGSAGFQ
LQHNIAGHETGFIARKSSGKQGPAIAFLAEYDALPGLGHACGHNLIGTISVAAAIALS
ETLEEIGGEVVVFGTPAEEGGPNGSAKSSYVKAGLFKNIDAALMIHPSGKTATTSPS
LAVDPLDFHFYGKTAHAAASPEEGINALDAVIQLYNSINALRQQLPSDVKIHGVITE
GGKAPNIIPDYAAARFFIRAATRKRCAEVTEKVKNIAQGAALATDTKVKIHQFQNEI
DELLVTKTYNDVVAEELELLGEDVNRKERFGIGSTDAGNVSQVVPTIHPYIKIGPDD
LIAHTNEFREAARSELGDKALITSAKALANTAYRLITEEGLLEKVKEEFREAQRNQG
BAS0167
Accession No. NC_005945, REGION: 166996 . . . 168963
Bacillus anthracis str. Sterne, complete genome.
Bases 1 to 1968
ORIGIN

SEQ ID NO: 125

| | |
|---|---|
| 1 | ttgatgaaag tgagtgaaaa aacatattta agtatagaag agattatttc attaccaacc |
| 61 | gtatcaagta caaatataag cgatgatggc aaaaatgtag catttgttaa gagaacagct |
| 121 | aactgggaag acaatacata tagaaaccat gtatggatat atgaaaaaga taaagggaag |
| 181 | agttatccac tgacaactgg agatatagat agtatacatc cattatggtc tccagattct |
| 241 | aagagcattg cttaccttag ctcaggtggt gacggggata tgaaaaatca gatctttgtt |
| 301 | aaatcactag acgactatag taaggttaaa attactgatg agaaagaggg aatcagtaat |
| 361 | tttaaatggg atcctaccgg taaaggtttt tattatatta cacagtcaaa agaatgtgag |
| 421 | gaaataaaga aacgtaagga gctatatgga gattttcaac atgtaggtaa ggaacatcag |
| 481 | aataattgtt tatgctacat tgaaatggaa aaagtgatac aaaatgataa agaggaacga |
| 541 | gagattaacg gtgtttatca attaactggt ggtaaggatt tttatatcca tggatttgat |
| 601 | atttcagata atgggaaaaa ggttgtatgt atggctacac caagcctaaa cgatcatatg |
| 661 | aatggtgatc tatatatatt agatgtcgaa gccagggaac tacaacagat gaatgtagat |
| 721 | aagttgttgg gcgggagcgc ttgtttttct ccagagggca acaaaatatg ttactcagca |
| 781 | agcataagag agaaggagta ttatagaaac catatacaag aaagtacatt agagatatat |
| 841 | gatatgaata ctggggaggt aattcagccc ttaacaaact ttgatagtat ggttatgcca |
| 901 | ttacagtgga cagctaaagg aattttaatt cgatggcagg acaagacgaa ttactttatt |
| 961 | ggtttgctag ctgaagatgg cacggtagaa acattaaggg aaaaagtaga tgggtttata |
| 1021 | atggatgcct ctataacaag agatggaaat catataacct ataataaggc tataacaaat |
| 1081 | gaaacctttg aaatctattt ggatgataaa aagataacga atgaaaatag ccttttcgag |
| 1141 | gggaagctta aaagtaacag ggaaatcatt tcatggcgaa gtagtgatgg tctggaaata |
| 1201 | gagggggttt tatcaacacc agtagagttt gacgcaaata aaaagtatcc tttattagta |
| 1261 | gtaattcacg gtggtccggc ttgggcatcc tttccgatat tttcaaactg cttcaatgag |
| 1321 | aaatatccga ttgaacagtt tgttgaaaag ggctttatcg ttttagagcc aaactataga |
| 1381 | ggaagttctg gttatggtaa tgaattttta aaagcaaact atagaaaaca aggacttgct |
| 1441 | gattacgatg atgttatatc tggagtggat gaactagttg aaaaagggat ggtagataaa |
| 1501 | gatagagtag gagttatggg atggagtaac ggaggatata tatcagcttt ctgttctaca |
| 1561 | tttagtagta gatttaaagc tatttcagtt gggggaggaa ttactaactg gagtactcat |
| 1621 | tatgtaaata cagatatccc ttactttatt agaatgtatt taggaaatac tccatggaat |
| 1681 | gatccagata tatataagag aacatcacca atgacatata ttaaatcagc ctgtacgcct |
| 1741 | accttaatcc aacatggcga aaaggatgca agaataccaa ttacaaatgc atatgaatta |
| 1801 | catcaagggt taaaggatat ggaagttgat acagaactca ttatatttaa aggaatggca |
| 1861 | tatagttctg accaaccagg agttcatgtg gctattatga agcagaactt gatgtggttt |
| 1921 | tcacattata ttcttggaga aagtatggag gattttagta ctatataa |

BAS0167
Accession No. NC_005945
Bacillus anthracis str. Sterne, complete genome.

SEQ ID NO: 126

MMKVSEKTYLSIEEIISLPTVSSTNISDDGKNVAFVKRTANWEDNTYRNHVWIYEK
DKGKSYPLTTGDIDSIHPLWSPDSKSIAYLSSGGDGDMKNQIFVKSLDDYSKVKITD
EKEGISNFKWDPTGKGFYYITQSKECEEIKKRKELYGDFQHVGKEHQNNCLCYIEM
EKVIQNDKEEREINGVYQLTGGKDFYIHGFDISDNGKKVVCMATPSLNDHMNGDL
YILDVEARELQQMNVDKLLGGSACFSPEGNKICYSASIREKEYYRNHIQESTLEIYD
MNTGEVIQPLTNFDSMVMPLQWTAKGILIRWQDKTNYFIGLLAEDGTVETLREKVD
GFIMDASITRDGNHITYNKAITNETFEIYLDDKKITNENSLFEGKLKSNREIISWRSSD
GLEIEGVLSTPVEFDANKKYPLLVVIHGGPAWASFPIFSNCFNEKYPIEQFVEKGFIV
LEPNYRGSSGYGNEFLKANYRKQGLADYDDVISGVDELVEKGMVDKDRVGVMG
WSNGGYISAFCSTFSSRFKAISVGGGITNWSTHYVNTDIPYFIRMYLGNTPWNDPDI
YKRTSPMTYIKSACTPTLIQHGEKDARIPITNAYELHQGLKDMEVDTELIIFKGMAY
SSDQPGVHVAIMKQNLMWFSHYILGESMEDFSTI
BAS4706
Accession NO. NC_005945, REGION: complement(4591713 . . . 4593566)
Bacillus anthracis str. Sterne, complete genome.
Bases 1 to 1854
ORIGIN

SEQ ID NO: 127

| | |
|---|---|
| 1 | atgaaaaaag ggattgtact aaagttattt accctcacaa cagcgctatg tatgttaatt |
| 61 | ttagcgacga tttttattgg acaaacgata ttttttaaac aatattatgc gaatcgaaaa |
| 121 | gtagaagata ttaaagtaaa tttaaattcc tttgagaaga attatttaaa ctatgcagga |
| 181 | aatacagaag aaattaagcg actggagcaa gactttttaa gagaaaataa tacgtggatt |
| 241 | acgacattag atcagaacgg gaatttaaag catgcggatg atttttattt tgaggttacg |
| 301 | atagatcgca agcaacagaa gagttttgga caacaaatat tcagaattcc tttatataat |
| 361 | ctcatcaata tagaagagat tgataataaa tcatcaaatc cgtttttagg ccaagaaatt |
| 421 | tatttttctg gagtgaaaaa agaagaaagt tttattccat tctctttttc gttaggtaag |
| 481 | caaaatttga atggatctaa taaaccgtta gaaaaagcat ttaatgagaa aatgagtaaa |
| 541 | ttagatcagg agaaaaagaa agcagctgag gaacaattcg gtaaggaaaa gaagccggta |
| 601 | gttcaggagc aagctgccca agaaccagat gttcacataa gagggcgtgt tacgaaagtt |
| 661 | cagcttccgg atgtaacagg tcctataaat ccaatttata aaaatagcat ctttttagat |
| 721 | aacataaaag aatttcaaac ggatttatta ttgaaagaga gcaagcacat acaatatgca |
| 781 | acacaaacaa tggactatga aaaaaatgat attaaatata aattattaat caaaccaata |
| 841 | aaagaaaaag acgaatccgt cacatatata tatgcgatgg cttctttaca gcctgtagat |
| 901 | gaagctgtac aaatggtgca agattactac atttacatca ttgcatttgt agttgttctt |
| 961 | atttttctag cttcgttcta ttactctaag caaatcgcaa agccgttatt aaaaataaat |
| 1021 | gatacaacga agaaaatagc gcatttagat ttcacagaac aaataccgat tacttcaaaa |
| 1081 | gatgaaattg gtgatttatc gaaaaatatt aatacactct ctaacaaatt aaattcccat |
| 1141 | attggacagc tagaacaaga tattgaaaag gaaagaaagt tagaaaagac gcggaaagaa |
| 1201 | ttcatttctg gtgtttcgca tgaactaaaa acaccgctga gtattatgaa aagctgtatt |
| 1261 | tctattttaa aagatggagt agctgagcat aaaaaagagt actattttca agcgatggaa |
| 1321 | cgggaagtag acaaaatgga tactttaatt ttggatatgc tggagttagc taaatttgag |
| 1381 | tcaggcacat ataaaatgaa aaaggaccca ttttatatcg atacagtaat ggaagctata |
| 1441 | tgtgaacacc tttctgtaga aatagagaaa aaagaacttc gtgttcataa acatataggt |
| 1501 | ccatttgaag tagtcgcaaa tcaaggccgg attgaacaag tcatcgtgaa cttcattacg |
| 1561 | aatgcgatac gttatacacc aaataaagaa gatattatta tttctacaat agatgagaag |
| 1621 | gatcatataa aagtatgtat tgaaaataaa ggtactcaca ttgaagaaga gcaattagat |
| 1681 | aaaatttggg atcgttttta tcgcgtggat gcagctcgcc agcgttcgca aggaggaaca |
| 1741 | ggtcttgggc ttgccatttc aaagaatatt ttagaactac atgatgctga atatggggca |
| 1801 | gaaaatacag aagatggtgt gttattttat ttctatttac cgaaaaaagc gtag |

BAS4706
Accession NO. NC_005945
Bacillus anthracis str. Sterne, complete genome.

SEQ ID NO: 128

MKKGIVLKLFTLTTALCMLILATIFIGQTIFFKQYYANRKVEDIKVNLNSFEKNYLNY
AGNTEEIKRLEQDFLRENNTWITTLDQNGNLKHADDFYFEVTIDRKQQKSFGQQIFR
IPLYNLINIEEIDNKSSNPFLGQEIYFSGVKKEESFIPFSFSLGKQNLNGSNKPLEKAFN
EKMSKLDQEKKKAAEEQFGKEKKPVVQEQAAQEPDVHIRGRVTKVQLPDVTGPIN
PIYKNSIFLDNIKEFQTDLLLKESKHIQYATQTMDYEKNDIKYKLLIKPIKEKDESVT
YIYAMASLQPVDEAVQMVQDYYIYIIAFVVVLIFLASFYYSKQIAKPLLKINDTTKKI
AHLDFTEQIPITSKDEIGDLSKNINTLSNKLNSHIGQLEQDIEKERKLEKTRKEFISGVS
HELKTPLSIMKSCISILKDGVAEHKKEYYFQAMEREVDKMDTLILDMLELAKFESGT
YKMKKDPFYIDTVMEAICEHLSVEIEKKELRVHKHIGPFEVVANQGRIEQVIVNFITN
AIRYTPNKEDIIISTIDEKDHIKVCIENKGTHIEEEQLDKIWDRFYRVDAARQRSQGGT
GLGLAISKNILELHDAEYGAENTEDGVLFYFYLPKKA
BAS4548
Accession No. NC_005945, REGION: complement(4455432 . . . 4456274)
Bacillus anthracis str. Sterne, complete genome.
Bases 1 to 843
ORIGIN

SEQ ID NO: 129

| | |
|---|---|
| 1 | atggatttgg aggcagtacg atcgtttatt gaagtgaaga atacgcgaag tttatcaaag |
| 61 | gcgagtaaga ttttacatat ttctcagccg gcgcttagta aacaaattca aaggttagaa |
| 121 | gcggatttag aggttacttt attgaagcgt tccgcgcaag gagtagaatt aacaagtgct |
| 181 | ggagagttat ttataaaaag aatgttaccg gttttggaac acatacatga agtgaaagct |
| 241 | gaaatgaaga agtttcaaga gaagaggaac atttcgatag gcatattgcc aagtttagca |
| 301 | gcacattaca tatcaaaatg taaagatata ttaggtgacg ggtttgaagt agaatggaaa |
| 361 | attgagcata caaaagtact gatgggactg tttaaagaac ggaagattga agcgatcttc |
| 421 | atcgattcag tagtagaagg cgctacgtgt ataaaagaaa tacgagaaga aaaaattgtt |
| 481 | tgtgtcgttt caaatgatca tctttataaa gagaaaacag taatccgaat ggaagatttg |
| 541 | caaaatgaaa agttaatcgt atacccagaa atctgtgatg ttaggaaaat gattacgcat |
| 601 | atgtttcaag ggataggtgc gaaaccgatc attgcatttg aaacttctta tgcagaaccg |
| 661 | atgcttgcaa tggttggtgc tggacttggt atcacgttac ttccagaaat gtcagtagag |
| 721 | caagcggtaa agcaagggaa tgtgcatgcg atttctattg aaccgccact tgtgcgcaaa |
| 781 | atttatttcg tatctcatat ggaagaaggc cctttgttgc attcatttga tgatgagagg |
| 841 | taa |

BAS4548
Accession No. NC_005945
Bacillus anthracis str. Sterne, complete genome.

SEQ ID NO: 130

MDLEAVRSFIEVKNTRSLSKASKILHISQPALSKQIQRLEADLEVTLLKRSAQGVELT
SAGELFIKRMLPVLEHIHEVKAEMKKFQEKRNISIGILPSLAAHYISKCKDILGDGFE
VEWKIEHTKVLMGLFKERKIEAIFIDSVVEGATCIKEIREEKIVCVVSNDHLYKEKTV
IRMEDLQNEKLIVYPEICDVRKMITHMFQGIGAKPIIAFETSYAEPMLAMVGAGLGIT
LLPEMSVEQAVKQGNVHAISIEPPLVRKIYFVSHMEEGPLLHSFDDER
BAS3747
Accession No. NC_005945, REGION: complement(3717093 . . . 3717599)
Bacillus anthracis str. Sterne, complete genome.
Bases 1 to 507
ORIGIN

SEQ ID NO: 131

| | |
|---|---|
| 1 | gtgccgttaa caccattaga tattcataac aaagaatttg gtcgcggatt ccgtggctat |
| 61 | gatgaggatc aagtaaatga gtttcttgat caaattatca aagattatga attagtcatt |
| 121 | cgtgagaaaa aagctttaga agaaaaagtt gcgcaattag aagggaaatt agatcatttc |
| 181 | tctaatattg aagatacgtt aaacaaatct atcgttgttg cacaagaagc agcggaagaa |
| 241 | gtaaaacgta atgcgcaaaa agaagcaaaa ttaatcgtac gtgaagctga aaagaatgca |
| 301 | gaccgtatta ttaatgaagc gttagtaaaa tcaagaaaag ttgctttcga tattgaagag |
| 361 | ctaaagaaac aagcgaaagt attccgcact cgtttccgta tgttattaga aacacagctt |
| 421 | gaaatgttaa acaacgatga ttgggataaa ctaattgagt tagaagacga agtagatgag |
| 481 | ctgttgaaaa aagaagaaac agtgtaa |

BAS3747
Accession No. NC_005945
Bacillus anthracis str. Sterne, complete genome.

SEQ ID NO: 132

MPLTPLDIHNKEFGRGFRGYDEDQVNEFLDQIIKDYELVIREKKALEEKVAQLEGKL
DHFSNIEDTLNKSIVVAQEAAEEVKRNAQKEAKLIVREAEKNADRIINEALVKSRKV
AFDIEELKKQAKVFRTRFRMLLETQLEMLNNDDWDKLIELEDEVDELLKKEETV
BAS0803
Accession No. NC_005945, REGION: complement(850135 . . . 852132)
Bacillus anthracis str. Sterne, complete genome.
Bases 1 to 1998
ORIGIN

SEQ ID NO: 133

| | |
|---|---|
| 1 | gtgaataaaa aaatggagca attgaagcac cctgttacgg ataatcaacg caaacatgct |
| 61 | ttaacaacaa accaaggagt aaaaatagcc gaggatgaat tttctttaaa gatgggctta |
| 121 | agaggcccaa ccttaatgga ggatttccac tttcgcgaga aaatgaccca ttttgatcac |
| 181 | gagcgaattc ccgaaagaat tgttcatgca cgcggagttg gcgctcatgg ttattttcag |
| 241 | ctttatgaat cgttagaagc atatacgaaa gcagattttc ttactaatcc ttccaagaaa |
| 301 | acacctgtat ttgtacgctt ctcaaccgtt caaggttcta aaggatccaa cgacgcagta |
| 361 | agagatgtac gcggattcgc cacaaaattt tatacagatg aaggaaacta cgatttagta |
| 421 | ggtaataata tgccagtatt ctttatccaa gatgctatta aatttcctga ttttgtccat |
| 481 | gcagttaaac cagaaccaca taatgatatt cctcagggag ctagcgctca tgatacattt |
| 541 | tgggattttg ttgcgcataa tcccgagtct actcatatgg ttatgtggca gatgagtgat |
| 601 | cgcgcaattc cacgtagttt acgaatgatg gaaggttttg gggttcatac atttcgactt |
| 661 | attaataaag aaggaaaagc ccacttcgta aaatttcact ggaaacctgc tcttggagtt |
| 721 | cattcattag tgtgggatga ggctcaaaag attgctggga aaaatcctga tttccatcgt |
| 781 | caagatttat atgaagcaat tgaaaaaggg gattatccgg aatgggagct cggattacag |
| 841 | ctaattccag aagaagatga gcatacattt gattttgata ttttagatcc aacgaaatta |
| 901 | tggccggaag aagaagttcc tgtaaaacta gttggtaaaa tgacattgaa caaaaatgtt |
| 961 | gataacttct ttgcggaaac ggagcaggta gctttccacc ctggtcatgt cgtaccaggt |
| 1021 | attgattttt caaatgaccc tcttctgcaa ggaagattat tctcctatac agatacacaa |
| 1081 | ttatctcgtt taggaggccc taatttccat caaaccccta ttaaccagcc cgtatgtcct |
| 1141 | tttcataata atcaacgtga tggtatgcat caaatgcaaa ttcatcgcgg tcaaacaagc |
| 1201 | tatcatccta atggcttaaa tgacaatcaa ccagcaaccg tgcaagctga acagggcgga |
| 1261 | tatgagcatt atcaagaaaa gattgatgga cataaaatta gggggcgcag taaaagcttc |
| 1321 | cttaactttt attcgcaagc taaacttttc tacaatagta tgagccctat tgaaaagcag |
| 1381 | catataaaag aagctttttg ctttgaagtt gggaagtgta aatcagacat ggtgaaggca |
| 1441 | aatgtaattg ctctactgaa tcatgtcgat cgtcaattag cacaagaagt cgctaacatt |
| 1501 | attggagctc ccttgcctaa ggaaaatcac gaagtaaatt cagatgcaaa atcacctgca |
| 1561 | ctaagtatgt ccaatactat ttttaaagct gattctaaaa atgttgcaat tgtattaaat |
| 1621 | ggtgacccaa gcgtctccct tctgtccgaa tggattcaag cttttgcaca gcatcgaatt |
| 1681 | aactatagca tcgtagataa taaaatttat cagttcaata attccattaa agtaactgat |
| 1741 | acttatacaa caacagactc ttcccttttt gatgcagtat tagtgtttag tagcgaatct |
| 1801 | gccattcacc cccctgtttt agaatttgca gaaactactt ttaaacattt taaaccgata |
| 1861 | gctcatgtcc ttagtaatcc tcatgctttg gacaatagta aaattaaatt agatggagca |
| 1921 | ggaatttaca acttagcaaa cacttcaatt gaatcattta tagaaggcat tgcccaagga |
| 1981 | cgattttgga atcgataa |

BAS0803
Accession No. NC_005945
Bacillus anthracis str. Sterne, complete genome.

SEQ ID NO: 134

MNKKMEQLKHPVTDNQRKHALTTNQGVKIAEDEFSLKMGLRGPTLMEDFHFREK
MTHFDHERIPERIVHARGVGAHGYFQLYESLEAYTKADFLTNPSKKTPVFVRFSTV
QGSKGSNDAVRDVRGFATKFYTDEGNYDLVGNNMPVFFIQDAIKFPDFVHAVKPE
PHNDIPQGASAHDTFWDFVAHNPESTHMVMWQMSDRAIPRSLRMMEGFGVHTFR
LINKEGKAHFVKFHWKPALGVHSLVWDEAQKIAGKNPDFHRQDLYEAIEKGDYPE
WELGLQLIPEEDEHTFDFDILDPTKLWPEEEVPVKLVGKMTLNKNVDNFFAETEQV
AFHPGHVVPGIDFSNDPLLQGRLFSYTDTQLSRLGGPNFHQTPINQPVCPFHNNQRD
GMHQMQIHRGQTSYHPNGLNDNQPATVQAEQGGYEHYQEKIDGHKIRGRSKSFLN
FYSQAKLFYNSMSPIEKQHIKEAFCFEVGKCKSDMVKANVIALLNHVDRQLAQEVA
NIIGAPLPKENHEVNSDAKSPALSMSNTIFKADSKNVAIVLNGDPSVSLLSEWIQAFA
QHRINYSIVDNKIYQFNNSIKVTDTYTTTDSSLFDAVLVFSSESAIHPPVLEFAETTFK
HFKPIAHVLSNPHALDNSKIKLDGAGIYNLANTSIESFIEGIAQGRFWNR
BAS2069
Accession No. NC_005945, REGION: 2078303 . . . 2078743
Bacillus anthracis str. Sterne, complete genome.
Bases 1 to 441
ORIGIN

SEQ ID NO: 135

| | |
|---|---|
| 1 | atgagaatat atgaggcaac aattgcagat ttagatggac tagcatcagt ttttaataac |
| 61 | tatcgtatgt tttatagaca agattccgat atagaaggag caaaagtatt tttacgaaat |
| 121 | cgaatggaga gaaaagaatc cgttattttc gtggcagttg aagacggtga atatattggg |
| 181 | ttcacacaat tatacccatc attttcttct atttcgatga aagaattatg gattttaaat |
| 241 | gatttatttg tgcaagccgc taagcgcgga gcaggaacag gaaaaaaatt attagaagct |
| 301 | gcgaaagaat ttgccttaga aaatggtgca aaaggtgtaa aattacaaac agagattgat |
| 361 | aatttatcag cgcagcgatt atatgctgaa aatggatatt tgagagataa tcgttatttc |
| 421 | cattatgaat taacgtttta a |

BAS2069
Accession No. NC_005945
Bacillus anthracis str. Sterne, complete genome.

SEQ ID NO: 136

MRIYEATIADLDGLASVFNNYRMFYRQDSDIEGAKVFLRNRMERKESVIFVAVEDG
EYIGFTQLYPSFSSISMKELWILNDLFVQAAKRGAGTGKKLLEAAKEFALENGAKG
VKLQTEIDNLSAQRLYAENGYLRDNRYFHYELTF
BAS2071
Accession No. NC_005945, REGION: complement(2078975 . . . 2079334)
Bacillus anthracis str. Sterne, complete genome.
Bases 1 to 360
ORIGIN

SEQ ID NO: 137

| | |
|---|---|
| 1 | atgacaagca ttcaagcaat tttcattgat cgtgacggta caattggcgg cgacactaca |
| 61 | atacattatc caggatcatt tacattattt ccttttacaa aagcatcact gcaaaaatta |
| 121 | aaagctaatc atataaaagt tttctctttc acaaatcaac caggtatcgc ggatgggata |
| 181 | gcgactatag ccgatttttc acaagaatta aaaagcttcg gttttgatga tatttacgta |
| 241 | tgtcctcaca aacacagtga tggttgtgaa tgccggaaac caagtacagg tatgcttctg |
| 301 | caagcagcgg aaaaacatgg gcttgattta acacaatgtg ctgtaattgg taattggtga |

BAS2071
Accession No. NC_005945
Bacillus anthracis str. Sterne, complete genome.

SEQ ID NO: 138

MTSIQAIFIDRDGTIGGDTTIHYPGSFTLFPFTKASLQKLKANHIKVFSFTNQPGIADGI
ATIADFSQELKSFGFDDIYVCPHKHSDGCECRKPSTGMLLQAAEKHGLDLTQCAVI
GNW
BAS4590
Accession No. NC_005945, REGION: complement(4491970 . . . 4492479)
Bacillus anthracis str. Sterne, complete genome.
Bases 1 to 510
ORIGIN

SEQ ID NO: 139

| | |
|---|---|
| 1 | atgaaagtag tagttggatc aaagaataaa acgaaggttg gagctgtgga gaaggtttgg |
| 61 | aaagatgccg aaattacatc tctttctgtt ccttcgggag tagcagcaca accgttttca |
| 121 | gatgaagaga cgatgcaagg agcaatcaat agagcgaagc gagcgctaga ggaaggagat |
| 181 | gctcaaattg gtattggact agaaggcggt gtaatgaaaa cggagcacgg tttatttatg |
| 241 | tgtaactggg gggcgctagc aacaagtgat ggtaaaatat ttgttgccgg cggggcacgt |
| 301 | attacgttac cggatgattt tttagcacct cttgaagagg gcaaggagtt aagtgaagtg |
| 361 | atggaagagt ttgtacagag aaaagatatt cgtagtcacg aaggtgcaat cggtattttt |
| 421 | acagatgatt atgtcgatcg cacggaatta tttgtacacg ttgttaagtt acttgttggg |
| 481 | caatataagt atgacgaaaa gcaagcataa |

BAS4590
Accession No. NC_005945
Bacillus anthracis str. Sterne, complete genome.

SEQ ID NO: 140

MKVVVGSKNKTKVGAVEKVWKDAEITSLSVPSGVAAQPFSDEETMQGAINRAKR
ALEEGDAQIGIGLEGGVMKTEHGLFMCNWGALATSDGKIFVAGGARITLPDDFLAP
LEEGKELSEVMEEFVQRKDIRSHEGAIGIFTDDYVDRTELFVHVVKLLVGQYKYDE
KQA
BAS3944
Accession No. NC_005945, REGION: complement(3891311 . . . 3892090)
Bacillus anthracis str. Sterne, complete genome.
Bases 1 to 780
ORIGIN

SEQ ID NO: 141

| | |
|---|---|
| 1 | atgaaagtcg catgtattca aatggatatt ttctttggag atgtagaaaa aaatattgag |
| 61 | aatgctaaaa ataaaataag cgaagcaatg aaggaaagac cagatgttat cgtcttacca |
| 121 | gaactatgga caacaggtta tgatttaacg agactttctg aaattgcaga tagggatgga |
| 181 | ttggaaacga aagaaaagtt gatagaatgg tcgaaacaat atggtgtaca tattgttggt |
| 241 | ggttctatag caaagcaaac agaacaaggt gttacaaata caatgtatgt tgtaacgaat |
| 301 | aaaggagaac tagttaatga atacagtaaa gtacatttat ttcagcttat ggatgaacat |
| 361 | aaatatttaa tcgctggaaa tagtacaggt gaatttaagt tagatgatgt agagtgtgcc |
| 421 | ggcacaattt gttatgacat tcgttttcca gagtggatgc gtgttcatac tgctaaaggt |
| 481 | gcaaaagttt tatttgttgt agctgaatgg ccattagttc gtttagcaca ttggcgtttg |
| 541 | ctattacaag caagagcggt tgaaaatcag tgttatgttg ttgcatgtaa tagggcagga |
| 601 | aaagatccaa ataatgagtt tgctggtcat tctttaattg tcgacccttg gggcgaagtt |
| 661 | gttgtagaag cgaatgaaga agaatcaatt ttatttggag agcttacatt cgagaaaatt |
| 721 | gaagaagtac gtaaaggaat tccagttttt gcagatcgtc gtccagaatt atacaaataa |

BAS3944
Accession No. NC_005945
Bacillus anthracis str. Sterne, complete genome.

SEQ ID NO: 142

MKVACIQMDIFFGDVEKNIENAKNKISEAMKERPDVIVLPELWTTGYDLTRLSEIAD
RDGLETKEKLIEWSKQYGVHIVGGSIAKQTEQGVTNTMYVVTNKGELVNEYSKVH
LFQLMDEHKYLIAGNSTGEFKLDDVECAGTICYDIRFPEWMRVHTAKGAKVLFVV
AEWPLVRLAHWRLLLQARAVENQCYVVACNRAGKDPNNEFAGHSLIVDPWGEVV
VEANEEESILFGELTFEKIEEVRKGIPVFADRRLPELYK
BAS2375
Accession No. NC_005945, REGION: 2374896 . . . 2376437
Bacillus anthracis str. Sterne, complete genome.
Bases 1 to 1542
ORIGIN

SEQ ID NO: 143

| | |
|---|---|
| 1 | atgatagacc aaaaacaaca atcgaataca tttgaagaac gagttgaaac gattaaacga |
| 61 | ggcggcgcac caaaatatca tgaacaaaat aaagcgaaag gtaaactatt cgttcgagat |
| 121 | cgcttagctc ttttatttga taatggtgaa tatgtagaag atgcattatt tgcaaattgt |
| 181 | gaagaaacgg gattacctgc tgatggtgtt ataacggcaa cgggaaaaat acatggtcgt |
| 241 | actgcatgcg taatggcaaa tgattctacg gtaaaggctg gatcatgggg cgcacgtaca |
| 301 | gttgaaaaga ttttacgtat tcaagaaacg gcagaaaaat tacgtgttcc gttattttat |
| 361 | ttagttgact ctgctggggc gcgtattacg gatcaagttg aaatgttccc agggcgccgc |
| 421 | ggtgcaggaa gaatcttcta taatcaagtg aaattatcag gtaaagttcc gcaagtatgt |
| 481 | ttattatttg gaccttctgc agctggtggc gcatatattc cagccttttg tgacgttgtt |
| 541 | atgatggtag aagggaatgc ttctatgtat ttaggatctc ctcgtatggc tgagatggtt |
| 601 | atcggtgaaa aggtaacttt agaagagatg ggcggagctc gtatgcattg ctctatatca |
| 661 | ggatgtggag atgttttatg taaaacagaa gaagatgcga ttacacaagc aagacaatac |
| 721 | atttcatatt ttccaaacaa ctacttagaa aagactccat tggttacacc tcaagaaccg |
| 781 | aaacaattcg ataaaacgtt agaacaaatc attccagaaa atcaaaatgc tcctttcaat |
| 841 | atgaaagatc ttattaatag agttattgat gaaggttctt tctacgaagt gaaaaaatta |
| 901 | tttgctcaag aactcattac aggtttagca cgtattgatg gtaagccagt aggtattatt |
| 961 | gcaaatcaac cgcgaatgaa aggcggcgta ttattccacg attcagctga taaagcagcg |
| 1021 | aagtttataa atttatgcga tgcatatcat attccgttat tattccttgc agatgtacct |
| 1081 | ggatttatga ttggtacaaa agtagaacgt gctggtatta ttcgtcacgg tgcaaaaatg |
| 1141 | atttctgcaa tgagtgaagc aactgtaccg aaaatttcta tcgttgttcg taaagcatat |
| 1201 | ggtgctggtt tatatgcgat ggcaggtcca gcctttgaac cagattgctg cctagcatta |
| 1261 | ccgacagcct ctattgcggt aatgggtcca gaagccgcgg tcaatgctgt atatgcaaat |
| 1321 | aagattgcag ctttacctga agaagagcgt gatagcttca ttgctgaaaa acgagaagag |
| 1381 | tataagaaag atattgatat ttaccattta gcatcagaga tggtcattga tggtattgtt |
| 1441 | catccaaaca atttaagaga agagttaaaa ggacgattcg aaatgtatat gagtaaatat |
| 1501 | caagtattta cggatcgtaa acatcctgtt tatccagttt aa |

BAS2375
Accession No. NC_005945
Bacillus anthracis str. Sterne, complete genome.

SEQ ID NO: 144

MIDQKQQSNTFEERVETIKRGGAPKYHEQNKAKGKLFVRDRLALLFDNGEYVEDA
LFANCEETGLPADGVITATGKIHGRTACVMANDSTVKAGSWGARTVEKILRIQETA
EKLRVPLFYLVDSAGARITDQVEMFPGRRGAGRIFYNQVKLSGKVPQVCLLFGPSA
AGGAYIPAFCDVVMMVEGNASMYLGSPRMAEMVIGEKVTLEEMGGARMHCSISG
CGDVLCKTEEDAITQARQYISYFPNNYLEKTPLVTPQEPKQFDKTLEQIIPENQNAPF
NMKDLINRVIDEGSFYEVKKLFAQELITGLARIDGKPVGIIANQPRMKGGVLFHDSA
DKAAKFINLCDAYHIPLLFLADVPGFMIGTKVERAGIIRHGAKMISAMSEATVPKISI
VVRKAYGAGLYAMAGPAFEPDCCLALPTASIAVMGPEAAVNAVYANKIAALPEEE
RDSFIAEKREEYKKDIDIYHLASEMVIDGIVHPNNREELKGRFEMYMSKYQVFTDR
KHPVYPV
BAS0485
Accession No. NC_005945, REGION: complement(509667 . . . 510797)
Bacillus anthracis str. Sterne, complete genome.
Bases 1 to 1131
ORIGIN

SEQ ID NO: 145

| | |
|---|---|
| 1 | atgaaaatat tgctcaaaca agccatggtc tatcctatta catcccaaaa atttcaaggg |
| 61 | gatgtactcg ttataggaga aaaaattgct gaggtcaagc ctttcattca acctactcaa |
| 121 | gatatgacag ttatagatgc acgtgctctt catcttttac ctggatttat tgatgtccat |
| 181 | actcatcttg gtctctacga tgaaggtact ggttgggctg gcaatgatgc aaatgaaaca |
| 241 | tctgaagttt caacaccaca tatccgttct ttagacggaa tccacccttt ggatattgca |
| 301 | tttcaagatg ctgtacaaaa tggaattaca actgttcacg ttatgccagg aagtcaaaac |
| 361 | attattggtg gtacgacttg tgtaataaaa acagccggaa cttgtattga tcatatgatt |
| 421 | attcaagaac ctgctggctt aaagattgcc tttggcgaaa atcctaaaaa agtccatagt |
| 481 | aatggaacaa aagagtccat tacgcgtatg ggaattatgg gattacttcg ggaatcattt |
| 541 | tatgaagcac aacactacgg gcatgaagct gattttcgaa tgcttcctat tttaaaagca |
| 601 | ttacgccgcg aaatacccgt acgtatccac gctcaccgag cagatgatat tagttctgct |
| 661 | ctacgttttg caacagagtt caatctcgat ttacgtattg aacattgtac agaaggacac |
| 721 | tttattattg aggaactttc gaagcacaat ttgaaagttt cagttggccc cacgcttaca |
| 781 | cgccgttcta aaattgaact taaaaacaaa acatgggata cttaccatat attatcgaaa |
| 841 | aatggagtgg aagtttccat cacaacagat cacccctata cacccattca atatttaaat |
| 901 | ctttgtgctg ctgttgctgt aagggaagga ttagacgaaa aaactgcact agaaggaatc |
| 961 | actatatttc cagcacgaaa tttacgttta gaggatagaa ttggaagcat tgaggtcgga |
| 1021 | aaagacgctg atcttgtgct gtggacccat catcctttcc attatttagc caagcctgta |
| 1081 | ctaactatga ttgatggaaa aataatttac aaaaaaaata aaaaaaacta g |

BAS0485
Accession No. NC_005945
Bacillus anthracis str. Sterne, complete genome.

SEQ ID NO: 146

MKILLKQAMVYPITSQKFQGDVLVIGEKIAEVKPFIQPTQDMTVIDARALHLLPGFID
VHTHLGLYDEGTGWAGNDANETSEVSTPHIRSLDGIHPLDIAFQDAVQNGITTVHV
MPGSQNIIGGTTCVIKTAGTCIDHMIIQEPAGLKIAFGENPKKVHSNGTKESITRMGI
MGLLRESFYEAQHYGHEADFRMLPILKALRREIPVRIHAHRADDISSALRFATEFNL
DLRIEHCTEGHFIIEELSKHNLKVSVGPTLTRRSKIELKNKTWDTYHILSKNGVEVSIT
TDHPYTPIQYLNLCAAVAVREGLDEKTALEGITIFPARNLRLEDRIGSIEVGKDADLV
LWTHHPFHYLAKPVLTMIDGKIIYKKNKKN
BAS0484
Accession No. NC_005945, REGION: complement(508868 . . . 509425)
Bacillus anthracis str. Sterne, complete genome.
Bases 1 to 558
ORIGIN

SEQ ID NO: 147

| | |
|---|---|
| 1 | atgttaaaaa aacgcgactt acacgacagc cacgttctat acgagttaat ggtggatcca |
| 61 | gctgtcttcc cttttgtgcg tcaaaaggct tattcttatg aggaatattt atttttaacg |
| 121 | aaacaaacga ttgaagctga agagcgtggg gaattaattt cacgcacaat tttagatgaa |
| 181 | tggggtaacc ctatcggaac aattacttta tttgatgtgc aagaaaaagc tggattcctt |
| 241 | ggaacatggc ttggcaaacc ttatcatgga aaaggctaca ataaattggc aaaagattca |
| 301 | ttttttagcg aacttttcta cgagctagat attgaaacaa tctttatgcg tattcgcaaa |
| 361 | ataaatattc gttctattaa agctgcagag aaattacaat atgtaaatct agcaaatgaa |
| 421 | acaagaaaag ctgtttatga tgaaattaat gcgaatgaag aagtatataa cttatatgaa |
| 481 | attccaaaag atcaatatac acttgctaca atgcgcgata caacgttcca agatgcacat |
| 541 | cacttaaaag aagcataa |

BAS0484
Accession No. NC_005945
Bacillus anthracis str. Sterne, complete genome.

SEQ ID NO: 148

MLKKRDLHDSHVLYELMVDPAVFPFVRQKAYSYEEYLFLTKQTIEAEERGELISRTI
LDEWGNPIGTITLFDVQEKAGFLGTWLGKPYHGKGYNKLAKDSFFSELFYELDIETI
FMRIRKINIRSIKAAEKLQYVNLANETRKAVYDEINANEEVYNLYEIPKDQYTLATM
RDTTFQDAHHLKEA
BAS2700
Accession No. NC_005945, REGION: 2680749 . . . 2681951
Bacillus anthracis str. Sterne, complete genome.
Bases 1 to 1203
ORIGIN

SEQ ID NO: 149

| | |
|---|---|
| 1 | ttgatgactt acacgttagc aactagaatg aaagcattcc aatcttctat atttagtgaa |
| 61 | ttaggggcct ataaaaaaga aaaaattgca gcaggtcaca aaatgattga tttaagtatc |
| 121 | gggaatcctg atatgccacc tgctgatttc gtaagagaag aaatggtaca tacagcaaat |
| 181 | caaaaagaaa gctatggata cacattaagt ggtattcaag aatttcacga agctgtaact |
| 241 | gaatattaca acaacactca taatgttata ttaaatgccg ataaagaagt tttattatta |
| 301 | atggggtcac aagatggact cgttcattta cctatggttt atgcgaatcc gggagatatt |
| 361 | atattagttc ctgacccagg atatacagct tatgaaacag aaattcaaat ggccggtgca |
| 421 | acatcttact atatgccttt aaaaaaagaa aatgatttct tacctaactt agagctaatt |
| 481 | cctgaagaaa ttgcggatca agcgaagatg atgattttaa acttcccagg gaatcccgtt |
| 541 | ccagcaatgg ctcatgaaga tttctttaaa gaggtaatcg cattcgcgaa aaagcataac |
| 601 | attattgttg tccatgattt tgcttatgct gaattttatt ttgatggtaa taaaccaatt |
| 661 | agcttcctct ctgtgcctgg tgcgaaagat gttggcgtag aaatcaactc tttgtcaaaa |
| 721 | agttatagtt tagcaggtag ccgtattggt tatatgattg gtaatgaaga aattgtcggg |
| 781 | gcacttacac aatttaaatc taatacggat tacggagtgt ttttaccaat tcaaaaagcg |
| 841 | gcatgtgctg cactaagaaa tggtgctgct ttttgtgaga aaaaccgtgg tatttatcaa |
| 901 | gaacgtagag atgctttagt cgatgggttc cgaacatttg gctggaatgt tgaaaaacca |
| 961 | gctggcagta tgttcgtctg ggccgaaatt ccgaaagggt ggacttctat agacttcgct |
| 1021 | tatgcattaa tggatcgtgc gaatgtcgtt gtcacaccag gtcatgcatt cggacctcac |
| 1081 | ggagaagggt ttgtacgcat tgcactcgtt caagataaag tagtgttaca acaagttgtt |
| 1141 | gaaaacatta gaaatagtgg tattttcgcc cttgaaaaag tagatgaatt agttaaaaat |
| 1201 | tag |

BAS2700
Accession No. NC_005945
Bacillus anthracis str. Sterne, complete genome.

SEQ ID NO: 150

MMTYTLATRMKAFQSSIFSELGAYKKEKIAAGHKMIDLSIGNPDMPPADFVREEMV
HTANQKESYGYTLSGIQEFHEAVTEYYNNTHNVILNADKEVLLLMGSQDGLVHLP
MVYANPGDIILVPDPGYTAYETEIQMAGATSYYMPLKKENDFLPNLELIPEEIADQA
KMMILNFPGNPVPAMAHEDFFKEVIAFAKKHNIIVVHDFAYAEFYFDGNKPISFLSV
PGAKDVGVEINSLSKSYSLAGSRIGYMIGNEEIVGALTQFKSNTDYGVFLPIQKAAC
AALRNGAAFCEKNRGIYQERRDALVDGFRTFGWNVEKPAGSMFVWAEIPKGWTSI
DFAYALMDRANVVVTPGHAFGPHGEGFVRIALVQDKVVLQQVVENIRNSGIFALE
KVDELVKN
BAS2061
Accession No. NC_005945, REGION: 2070788 . . . 2071693
Bacillus anthracis str. Sterne, complete genome.
Bases 1 to 906
ORIGIN

SEQ ID NO: 151

| | |
|---|---|
| 1 | atggccgatt tgatagaaac agggaaatac atgaatatta gagggaaaaa gctatacgtt |
| 61 | gaaacgcatg gagatcctaa aaataaacca gttctatact tgcatggtgg accgggagaa |
| 121 | agttgctatg atttttcatt tcatcaagcg gaacgtttaa aagattcttt atatgtaatt |
| 181 | atgatagatc aaagaggtgt ttgtcgctca gaagaaatta ctgaagacga agcttttgga |
| 241 | ttaaatgatt tgattgaaga ctgtgaggaa ttaaaaaaag tattacaaat taagaagtgg |
| 301 | tctataattg gacactcttt cggtggatat ttagcattgc tatatgcgtc gatatatcca |
| 361 | ggttcaataa agaaaataat atttgaagga ccaacttttg attttgcatt aacaagcagg |
| 421 | gctttgttgc aaaagacagg gcatttatta aaaaagtatg gaaaagaaga agtagcagaa |
| 481 | gaatctcttg cttattcatc tagcaatgcg agttcagaag agttgctaga agcttatata |
| 541 | agactaagtg atgaactaga agaaaaaaga atggagattt acaattataa ggaagatggg |
| 601 | acagatgaga gtttatatag tgatgaagag tgggaagtat tttcaaatcg ctccaagatc |
| 661 | cattttgata gattaaaatt agagggagca tgtcatacgt cattattatc taaaataaaa |
| 721 | gatgtacaga atccaatgtt attaatagta ggaaaacatg atgtagtaac gtgtgaaaaa |
| 781 | caaattgaaa tatttaataa agatgctcga aacggcaagt atatcgtatt tgaagagagc |
| 841 | ggtcattcac ctcattatga ggaagcagat agattcgcag aaacagtcat acatttttta |
| 901 | aaatga |

BAS2061
Accession No. NC_005945
Bacillus anthracis str. Sterne, complete genome.

SEQ ID NO: 152

MADLIETGKYMNIRGKKLYVETHGDPKNKPVLYLHGGPGESCYDFSFHQAERLKD
SLYVIMIDQRGVCRSEEITEDEAFGLNDLIEDCEELKKVLQIKKWSIIGHSFGGYLAL
LYASIYPGSIKKIIFEGPTFDFALTSRALLQKTGHLLKKYGKEEVAEESLAYSSSNASS
EELLEAYIRLSDELEEKRMEIYNYKEDGTDESLYSDEEWEVFSNRSKIHFDRLKLEG
ACHTSLLSKIKDVQNPMLLIVGKHDVVTCEKQIEIFNKDARNGKYIVFEESGHSPHY
EEADRFAETVIHFLK
BAS4188
Accession No. NC_005945, REGION: 4104135 . . . 4104905
Bacillus anthracis str. Sterne, complete genome.
Bases 1 to 771
ORIGIN

SEQ ID NO: 153

| | |
|---|---|
| 1 | atgtttccaa aatcacccaa caggcaaatg tatccgcagc cagggcagca accttataca |
| 61 | ccatatccaa ttccacaact accaccgatg gcacaaaaaa agaaagggtt ccttgctaaa |
| 121 | ctctttaaaa aacacgatcc aaccgaacct ttcatgcaaa tggttccgcc ttatcgacaa |
| 181 | atggaaggac caccgccaat gatgcaccaa caacagcaac cgccacccca atatcgacag |
| 241 | caatatcaac aacaatatca acaacaatat caacaacaat atcaacaaca atacccgcaa |
| 301 | caatactcac agcaatacca accatacatg cagcatcatc ccgagcaaat gatccctcct |
| 361 | caaatgtatg aatcaaacga aacgcgcggc ggtgcagcaa ctacagcagc atcaagtagc |
| 421 | ggcatcggta gttttttttc gaatttaatt tcgaatccaa ctaatatgat aaataatatc |
| 481 | gaaaaagtat cacaagtcgt tcaatctgta agccctgtcg tcgaacagta cggtcccatt |
| 541 | atgcgtaacc taccaagcat cgttaaaatc ctcacctctg gaaaaagtac ggaagaaaat |
| 601 | ccaaccgaag atcaaactga agacctaaca gaaaaggttg aagtagcaac tccacctcct |
| 661 | ccacaaaaaa aaagaaaaag aaaaaaaatg gtgattgagc cagttataga aaaagaagtg |
| 721 | cgcgaggagc ctgttcaaaa aatagcaaca aaaccaaaac tatatgtgta a |

BAS4188
Accession No. NC_005945
Bacillus anthracis str. Sterne, complete genome.

SEQ ID NO: 154

MFPKSPNRQMYPQPGQQPYTPYPIPQLPPMAQKKKGFLAKLFKKHDPTEPFMQMV
PPYRQMEGPPPMMHQQQQPPPQYRQQYQQQYQQQYQQQYQQQYPQQYSQQYQP
YMQHHPEQMIPPQMYESNETRGGAATTAASSSGIGSFFSNLISNPTNMINNIEKVSQ
VVQSVSPVVEQYGPIMRNLPSIVKILTSGKSTEENPTEDQTEDLTEKVEVATPPPPQK
KRKRKKMVIEPVIEKEVREEPVQKIATKPKLYV
BAS3788
Accession No. NC_005945, REGION: complement(3753221 . . . 3755335)
Bacillus anthracis str. Sterne, complete genome.
Bases 1 to 2115
ORIGIN

SEQ ID NO: 155

| | |
|---|---|
| 1 | ttggaaaaaa tgaacggtga agccaaagtc ataaaaatgc ccgcaggtgg ttaccagag |
| 61 | tttgaaaact atgtagaagt agaaataact caagataatc aagatacttg gatttataaa |
| 121 | gattttacgg ttgttccagg tgattatatt atttctgcat ggattcaaag tacatggaat |
| 181 | agtattccag ctattggagt taaagataat gcacctggag cttccggtga tccaggggtt |
| 241 | aaatactttt tatttgatgc tcctcaaaca atgttggatg gtaaatggca ttacttccac |
| 301 | ggcaaattaa aagttgtgga tgggaaagtg cgtgtttact ttgggatgtt gaagacggga |
| 361 | aatgtaaaag gtgctaaaac aagaataacc ggagtgaaat ttgcgaatgg tactgtattg |
| 421 | gataactgga ctatcgctcc tgaagatatg acttataata cagagtttac taaaaagact |
| 481 | gctgaaatta ttacaagtgt tgataaggtt agttcaactc taacggagac aaataaacaa |
| 541 | gtagtagcag ttgaaaagaa agctgatata gctaatgaac aaataactac aacgaataag |
| 601 | aaggtttctg acgtatcaca aacagtagat ggattaaaag tgaatatatc agatatctca |
| 661 | aagatccaac aagggcatac aactgagtta caacaacata gctctaaaat agatgctaat |
| 721 | gcgaaagcta ttcaaacgaa ggttgatagt caatttgtcg aggagtatac aggtggatta |
| 781 | ggtagcactc agctgataag ggatgctgaa ttcgttgacg gattcaagta ttggtataaa |
| 841 | tctaataacg caaatttcac cgctgaagta gatacaacta atctatataa tggtagtcca |
| 901 | tcaatgcgtt taaaagggat taatcagacg gcgaacgtta acacaaatgt gacatctact |
| 961 | actaagatac ctgttacgcc aggagaaaaa atcacagctt ctttagccct atttacaaat |
| 1021 | aaattaagtg aacatatcaa tccttatcta acgtgtgctg ttgtttgttg ggatggtaat |
| 1081 | aatcaacaac taacagcgat ggggtggaat actaaggtag cagataatac ttggacgaaa |
| 1141 | acatcttata cagtaacagt gccggaaacc gctgtaagaa tggaattacg tgtatacgta |
| 1201 | acaagaaatg gagaattgtg gttctctaag ccgatgttac aacgcggtga ggtagcgagt |
| 1261 | tacttcacat tacaccctaa agattacact gactatgaca agttagttga tgaaatagcg |
| 1321 | cgtcgtgttt taacagagga ttacgataag aaaatgactg aaatgaacac tcagtttatc |
| 1381 | caaacttcta aagcattgga attaaaagca gaagcgaaaa atgtttatac aaaaaatgaa |
| 1441 | gttgatagta gagacgatgc ggtagtagaa actatgaatg ctcagtttaa agttcaagct |
| 1501 | gatgaaataa gctctaaggt gtccaaaggg agtgttattt catccataaa tcaaacggca |
| 1561 | gaaaaaatta aaatctcagc agatttaata gatctaattg gtaaagtaga agcatcttgg |
| 1621 | ttaagagcag gattactcat gggtatgacg ataaaaacaa gcaatacaaa tgaacatttg |
| 1681 | catatggaaa accaggtgat gcgttttgta aaccaagggt cagataagat ggtaatagga |
| 1741 | tttgaaaacg aaaggaaaag taaaactcga aatccataca tcatactagg tgaaggtgac |
| 1801 | ggatccggta aaaactttgg aagtatttat aaagatggta atggagttta ttatcgatac |
| 1861 | gtggatttga atggagcaga aagtaatatt cgtttaacga atgcaggtaa tattggtgtc |
| 1921 | acagcccaag atggtctttg gataaattct aaaagaacaa atttcagctc tgtaattgaa |
| 1981 | gtaccagcca ttagattcaa ctcactcggt actactccag gttcgcagca gggaaatttt |
| 2041 | tggattggca atggatataa aggatttgga gtatattact acgataatta ttggaagttc |
| 2101 | gtacaaggat cttaa |

BAS3788
Accession No. NC_005945
Bacillus anthracis str. Sterne, complete genome.

SEQ ID NO: 156

MEKMNGEAKVIKMPAGGLPEFENYVEVEITQDNQDTWIYKDFTVVPGDYIISAWIQ
STWNSIPAIGVKDNAPGASGDPGVKYFLFDAPQTMLDGKWHYFHGKLKVVDGKV
RVYFGMLKTGNVKGAKTRITGVKFANGTVLDNWTIAPEDMTYNTEFTKKTAEIITS
VDKVSSTLTETNKQVVAVEKKADIANEQITTTNKKVSDVSQTVDGLKVNISDISKIQ
QGHTTELQQHSSKIDANAKAIQTKVDSQFVEEYTGGLGSTQLIRDAEFVDGFKYWY
KSNNANFTAEVDTTNLYNGSPSMRLKGINQTANVNTNVTSTTKIPVTPGEKITASLA
LFTNKLSEHINPYLTCAVVCWDGNNQQLTAMGWNTKVADNTWTKTSYTVTVPET
AVRMELRVYVTRNGELWFSKPMLQRGEVASYFTLHPKDYTDYDKLVDEIARRVLT
EDYDKKMTEMNTQFIQTSKALELKAEAKNVYTKNEVDSRDDAVVETMNAQFKVQ
ADEISSKVSKGSVISSINQTAEKIKISADLIDLIGKVEASWLRAGLLMGMTIKTSNTNE
HLHMENQVMRFVNQGSDKMVIGFENERKSKTRNPYIILGEGDGSGKNFGSIYKDGN
GVYYRYVDLNGAESNIRLTNAGNIGVTAQDGLWINSKRTNFSSVIEVPAIRFNSLGT
TPGSQQGNFWIGNGYKGFGVYYYDNYWKFVQGS
BAS5123
Accession No. NC_005945, REGION: complement(5007091 . . . 5007480)
Bacillus anthracis str. Sterne, complete genome.
Bases 1 to 390
ORIGIN

SEQ ID NO: 157

| | |
|---|---|
| 1 | atgacagagc gattaaaaat tttattatat ggtgatgtag acttaaactt aatagatgga |
| 61 | tcagctatgt ggttagtttc attaactcaa gttttacatg aagatagaaa tattgatatt |
| 121 | gatatacttc agaagcgacc tattattaac aataagctgg tcaagccttt attaaaaaag |
| 181 | gaaagggtta attttattga tcctttctca tgtgctaaag ggagtgaagg atggtataaa |
| 241 | agaaaacgtc tacttgttga tgatgctatt caaaaaatta aagagcaaga gaagcagcat |
| 301 | aattatgatg tgattttagt tcgtgggttt gaattagcgt ataaactttc taaaatacca |
| 361 | tttttatcaa aaaaatatat tcgtatataa |

BAS5123
Accession No. NC_005945
Bacillus anthracis str. Sterne, complete genome.

SEQ ID NO: 158

MTERLKILLYGDVDLNLIDGSAMWLVSLTQVLHEDRNIDIDILQKRPIINNKLVKPLL
KKERVNFIDPFSCAKGSEGWYKRKRLLVDDAIQKIKEQEKQHNYDVILVRGFELAY
KLSKIPFLSKKYIRI
Protective Antigen
Accession No. AY997299
Bacillus anthracis strain A16R protective antigen (pag) gene, complete cds.
Bases 1 to 2295
ORIGIN

SEQ ID NO: 159

| | |
|---|---|
| 1 | atgaaaaaac gaaaagtgtt aataccatta atggcattgt ctacgatatt agtttcaagc |
| 61 | acaggtaatt tagaggtgat tcaggcagaa gttaaacagg agaaccggtt attaaatgaa |
| 121 | tcagaatcaa gttcccaggg gttactagga tactatttta gtgatttgaa ttttcaagca |
| 181 | cccatggtgg ttacctcttc tactacaggg gatttatcta ttcctagttc tgagttagaa |
| 241 | aatattccat cggaaaacca atattttcaa tctgctattt ggtcaggatt tatcaaagtt |
| 301 | aagaagagtg atgaatatac atttgctact tccgctgata atcatgtaac aatgtgggta |
| 361 | gatgaccaag aagtgattaa taaagcttct aattctaaca aaatcagatt agaaaaagga |
| 421 | agattatatc aaataaaaat tcaatatcaa cgagaaaatc ctactgaaaa aggattggat |
| 481 | ttcaagttgt actggaccga ttctcaaaat aaaaaagaag tgatttctag tgataactta |
| 541 | caattgccag aattaaaaca aaaatcttcg aactcaagaa aaaagcgaag tacaagtgct |
| 601 | ggacctacgg ttccagaccg tgacaatgat ggaatccctg attcattaga ggtagaagga |
| 661 | tatacggttg atgtcaaaaa taaaagaact tttctttcac catggatttc taatattcat |
| 721 | gaaaagaaag gattaaccaa atataaatca tctcctgaaa aatggagcac ggcttctgat |
| 781 | ccgtacagtg atttcgaaaa ggttacagga cggattgata agaatgtatc accagaggca |
| 841 | agacaccccc ttgtggcagc ttatccgatt gtacatgtag atatggagaa tattattctc |
| 901 | tcaaaaaatg aggatcaatc cacacagaat actgatagtc aaacgagaac aataagtaaa |
| 961 | aatacttcta caagtaggac acatactagt gaagtacatg gaaatgcaga agtgcatgcg |
| 1021 | tcgttctttg atattggtgg gagtgtatct gcaggattta gtaattcgaa ttcaagtacg |
| 1081 | gtcgcaattg atcattcact atctctagca ggggaaagaa cttgggctga aacaatgggt |
| 1141 | ttaaataccg ctgatacagc aagattaaat gccaatatta gatatgtaaa tactgggacg |
| 1201 | gctccaatct acaacgtgtt accaacgact tcgttagtgt taggaaaaaa tcaaacactc |
| 1261 | gcgacaatta aagctaagga aaaccaatta agtcaaatac ttgcacctaa taattattat |
| 1321 | ccttctaaaa acttggcgcc aatcgcatta aatgcacaag acgatttcag ttctactcca |
| 1381 | attacaatga attacaatca atttcttgag ttagaaaaaa cgaaacaatt aagattagat |
| 1441 | acggatcaag tatatgggaa tatagcaaca tacaattttg aaaatggaag agtgagggtg |
| 1501 | gatacaggct cgaactggag tgaagtgtta ccgcaaattc aagaaacaac tgcacgtatc |
| 1561 | atttttaatg gaaaagattt aaatctggta gaaaggcgga tagcggcggt taatcctagt |
| 1621 | gatccattag aaacgactaa accggatatg acattaaaag aagcccttaa aatagcattt |
| 1681 | ggatttaacg aaccgaatgg aaacttacaa tatcaaggga aagacataac cgaatttgat |
| 1741 | tttaatttcg atcaacaaac atctcaaaat atcaagaatc agttagcgga attaaacgca |
| 1801 | actaacatat atactgtatt agataaaatc aaattaaatg caaaaatgaa tattttaata |
| 1861 | agagataaac gttttcatta tgatagaaat aacatagcag ttggggcgga tgagtcagta |
| 1921 | gttaaggagg ctcatagaga agtaattaat tcgtcaacag agggattatt gttaaatatt |
| 1981 | gataaggata taagaaaaat attatcaggt tatattgtag aaattgaaga tactgaaggg |
| 2041 | cttaaagaag ttataaatga cagatatgat atgttgaata tttctagttt acggcaagat |
| 2101 | ggaaaaacat ttatagattt taaaaaatat aatgataaat taccgttata tataagtaat |
| 2161 | cccaattata aggtaaatgt atatgctgtt actaaagaaa acactattat taatcctagt |
| 2221 | gagaatgggg atactagtac caacgggatc aagaaaattt taatcttttc taaaaaaggc |
| 2281 | tatgagatag gataa |

Protective Antigen
Accession No. AY997299
Bacillus anthracis strain A16R protective antigen (pag) gene, complete cds.

SEQ ID NO: 160

MKKRKVLIPLMALSTILVSSTGNLEVIQAEVKQENRLLNESESSSQGLLGYYFSDLN
FQAPMVVTSSTTGDLSIPSSELENIPSENQYFQSAIWSGFIKVKKSDEYTFATSADNH
VTMWVDDQEVINKASNSNKIRLEKGRLYQIKIQYQRENPTEKGLDFKLYWTDSQN
KKEVISSDNLQLPELKQKSSNSRKKRSTSAGPTVPDRDNDGIPDSLEVEGYTVDVKN
KRTFLSPWISNIHEKKGLTKYKSSPEKWSTASDPYSDFEKVTGRIDKNVSPEARHPL
VAAYPIVHVDMENIILSKNEDQSTQNTDSQTRTISKNTSTSRTHTSEVHGNAEVHAS
FFDIGGSVSAGFSNSNSSTVAIDHSLSLAGERTWAETMGLNTADTARLNANIRYVNT
GTAPIYNVLPTTSLVLGKNQTLATIKAKENQLSQILAPNNYYPSKNLAPIALNAQDD
FSSTPITMNYNQFLELEKTKQLRLDTDQVYGNIATYNFENGRVRVDTGSNWSEVLP
QIQETTARIIFNGKDLNLVERRIAAVNPSDPLETTKPDMTLKEALKIAFGFNEPNGNL
QYQGKDITEFDFNFDQQTSQNIKNQLAELNATNIYTVLDKIKLNAKMNILIRDKRFH
YDRNNIAVGADESVVKEAHREVINSSTEGLLLNIDKDIRKILSGYIVEIEDTEGLKEVI
NDRYDMLNISSLRQDGKTFIDFKKYNDKLPLYISNPNYKVNVYAVTKENTIINPSEN
GDTSTNGIKKILIFSKKGYEIG

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. An immunogenic composition comprising at least one anthrax spore-associated protein or an immunogenic fragment thereof.

2. The composition of claim 1, further comprising protective antigen (PA) or an immunogenic fragment thereof.

3. An immunogenic composition comprising at least one expression vector, wherein the expression vector comprises a nucleic acid molecule encoding an anthrax spore-associated protein or an immunogenic fragment thereof.

4. The composition of claim 3, wherein the expression vector comprises at least one additional nucleic acid molecule encoding a second anthrax spore-associated protein or an immunogenic fragment thereof.

5. The composition of claim 3, further comprising a nucleic acid molecule encoding protective antigen (PA) or an immunogenic fragment thereof.

6. The composition of claim 1, wherein the composition is acellular.

7. The composition of claim 1, wherein the composition induces an immunological response in a subject against Bacillus anthracis.

8. The composition of claim 7, wherein the immunological response induced in the subject is against Bacillus anthracis in the spore form.

9. The composition of claim 2, wherein the composition induces an immunological response in a subject against Bacillus anthracis in the spore form and in the bacillus form.

10. The composition of claim 1, further comprising a pharmaceutically acceptable excipient.

11. The composition of claim 1, further comprising an adjuvant.

12. The composition of claim 1, wherein the expression vector is a viral vector or a plasmid vector.

13. The composition of claim 1, wherein the protein has an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158, and immunogenic fragments thereof.

14. The composition of claim 3, wherein the nucleic acid sequence is selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:11, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157, and fragments thereof.

15. The composition of claim 7, wherein the subject is a mammal.

16. The composition of claim 15, wherein the mammal is a human.

17. A method for inducing an immunological response in a subject comprising administering to said subject the immunogenic composition of claim 1.

18. The method of claim 17, wherein the subject is not infected with Bacillus anthracis.

19. The method of claim 17, wherein the immunological response is against Bacillus anthracis.

20. The method of claim 19, wherein the immunological response is against Bacillus anthracis in the spore form.

21. A method for inducing an immunological response in a subject comprising administering to said subject the immunogenic composition of claim 2.

22. The method of claim 21, wherein the subject is not infected with Bacillus anthracis.

23. The method of claim 21, wherein the subject is infected with Bacillus anthracis.

24. The method of claim 23, wherein the administering occurs about one to about sixty days after infection.

25. The method of claim 23, wherein the Bacillus anthracis has not yet germinated.

26. The method of claim 23, wherein an additional therapy against Bacillus anthracis infection is administered.

27. The method of claim 26, wherein the additional therapy is antibiotic therapy.

28. The method of claim 21, wherein the immunological response is against Bacillus anthracis in the spore form and in the bacillus form.

29. The method of claim 17, wherein the amount of immunological response is effective to confer substantial protective immunity against infection with Bacillus anthracis in the subject.

30. The method of claim 17, wherein the subject is a mammal.

31. The method of claim 30, wherein the mammal is a human.

32. The method of claim 17, wherein the immunogenic composition is administered 1 to 2 times.

33. A kit comprising the immunogenic composition of claim 1 and instructions for administering the immunogenic composition to induce an immunological response in a subject.

34. A vaccine comprising the immunogenic composition of claim 1.

35. The method of claim 1, further comprising obtaining said anthrax spore-associated protein or immunogenic fragment thereof or said nucleic acid molecule encoding an anthrax spore-associated protein or an immunogenic fragment thereof.

36. A method for inducing an immunological response in a subject comprising administering to said subject the immunogenic composition of claim 1.
